(19) United States
(12) Patent Application Publication
(10) Pub. No.: US 2026/0028384 A1
(43) Pub. Date: Jan. 29, 2026

(54) USE OF IL-2 VARIANT FOR TREATING KIDNEY TRANSPLANT REJECTION

(71) Applicants: Visterra, Inc., Waltham, MA (US); Duke University, Durham, NC (US)

(72) Inventors: David William Oldach, Waltham, MA (US); Stuart Johnston Knechtle, Durham, NC (US); Gregory Babcock, Waltham, MA (US); John J. O'Neil, Waltham, MA (US); Jean Kwun, Durham, NC (US)

(73) Assignees: Visterra, Inc., Waltham, MA (US); Duke University, Durham, NC (US)

(21) Appl. No.: 19/182,189

(22) Filed: Apr. 17, 2025

Related U.S. Application Data
(60) Provisional application No. 63/635,218, filed on Apr. 17, 2024.
(60) Provisional application No. 63/660,250, filed on Jun. 14, 2024.

(51) Int. Cl. C07K 14/55; A61K 38/00; A61P 37/06

(52) U.S. Cl. CPC C07K 14/55; A61P 37/06; A61K 38/00; C07K 2319/30

(57) ABSTRACT

The present invention provides, among other things, a method of prophylaxis for kidney rejection comprising administering to the recipient an alpha-biased IL-2 variant in conjunction with the kidney transplant. In another aspect, the present invention provides, among other things, a method of improving a kidney transplant in a recipient, comprising administering to the recipient an alpha-biased IL-2 variant in conjunction with the kidney transplantation at a therapeutically effective amount, wherein the improvement in the kidney transplant results in prolonged stability of the kidney transplant, survival of the recipient, and/or reduced rejection symptoms as compared to a control.

### CROSS-REFERENCE TO RELATED APPLICATIONS

This application claims priority to, and the benefit of, U.S. Provisional Application No. 63/635,218, filed Apr. 17, 2024, and U.S. Provisional Application No. 63/660,250, filed Jun. 14, 2024, the disclosure of each of which is hereby incorporated by reference in its entirety.

### INCORPORATION-BY-REFERENCE OF SEQUENCE LISTING

The present specification refers to and includes a Sequence Listing (submitted electronically as an XML file entitled SVI-013US1_SL.xml. The .xml file was generated on Jul. 29, 2025, and is 28,254 bytes in size. The entire contents of the Sequence Listing are herein incorporated by reference in its entirety.

### BACKGROUND

Kidney transplantation is a surgical procedure that involves placing a donated kidney from a living or deceased donor into a person whose own kidneys have lost the ability to properly filter waste and fluids from the body. This condition, known as kidney failure or end-stage renal disease (ESRD), can lead to the buildup of harmful levels of waste products in the body, as well as high blood pressure. Kidney transplantation is typically performed to treat kidney failure or ESRD, when the kidneys can no longer function effectively. This procedure offers the best chance to restore normal kidney function and improve the patient's quality of life. However, kidney transplantation faces long-term challenges, including graft failure and side effects from immunosuppressive treatments. Addressing these long-term challenges and improving overall outcomes are crucial priorities in the field of kidney transplantation.

### SUMMARY OF THE INVENTION

The present invention provides, among other things, a method of prophylaxis for kidney rejection comprising administering to the recipient an alpha-biased IL-2 variant in conjunction with the kidney transplant. As described herein, the present invention is, in part, based on an unexpected discovery that a transplanted kidney remained stable for over 235 days when the recipient was administered an alpha-biased IL-2 variant in conjunction with the kidney transplant. Further, the transplanted kidney remained stable even after discontinuing the treatment with the alpha-biased IL-2 variant and an immunosuppressive agent. This is significant because rejection of the transplanted kidney by the immune system is a major challenge along with increased risks of infections and malignancies caused by currently approved therapies such as immunosuppressants. As demonstrated herein, the administration of an IL-2 variant of the present invention prolonged survival of the recipient and stabilized the transplanted kidney such that the recipient, among other things, maintained the creatinine level and blood urea nitrogen level within the normal threshold.

In one aspect, the present invention provides, among other things, a method of prophylaxis for kidney rejection in a recipient receiving a kidney transplant comprising administering to the recipient an alpha-biased IL-2 variant in conjunction with the kidney transplant.

In one aspect, the present invention provides, among other things, a method of treating a kidney rejection in a recipient receiving a kidney transplant comprising administering to the recipient an alpha-biased IL-2 variant in conjunction with the kidney transplant.

In one aspect, the present invention provides, among other things, a method of improving kidney transplant in a recipient comprising administering to the recipient an alpha-biased IL-2 variant in conjunction with the kidney transplant.

In some embodiments, an alpha-biased IL-2 variant is administered at a therapeutically effective amount.

In some embodiments, the improvement in the kidney transplant results in prolonged stability of the kidney transplant, survival of the recipient, and reduced rejection symptoms as compared to a control. In some embodiments, the improvement in the kidney transplant results in prolonged stability of the kidney transplant. In some embodiments, the improvement in the kidney transplant results in survival of the recipient. In some embodiments, the improvement in the kidney transplant results in reduced rejection symptoms as compared to a control. In some embodiments, the improvement in the kidney transplant results in prolonged stability of the kidney transplant and reduced rejection symptoms as compared to a control. In some embodiments, the improvement in the kidney transplant results in survival of the recipient and reduced rejection symptoms as compared to a control. In some embodiments, the improvement in the kidney transplant results in prolonged stability of the kidney transplant and survival of the recipient.

In some embodiments, the control is historical data. In some embodiments, the control is a comparable recipient without the administration of the alpha-biased IL-2 variant. In some embodiments, the control is a comparable recipient receiving a standard of care. In some embodiments, the control is a comparable recipient receiving a standard of care without the administration of the alpha-biased IL-2 variant.

In some embodiments, the standard of care comprises induction phase and maintenance phase. In some embodiments, the standard of care comprises induction phase. In some embodiments, the standard of care comprises maintenance phase.

In some embodiments, the induction phase comprises administration of a T cell depleting agents. In some embodiments the T cell depleting agent is rabbit antithymocyte globulin (rATG).

In some embodiments, the maintenance phase comprises administration of one or more immunosuppressive agents. In some embodiments, the one or more immunosuppressive agents comprise calcineurin inhibitors (CNI), mammalian target of rapamycin (mTOR) inhibitors, antiproliferative agents, and/or corticosteroids. In some embodiments, the immunosuppressive agent is a CNI. In some embodiments, the one or more immunosuppressive agent is a mTOR inhibitor. In some embodiments, the one or more immunosuppressive agent is an antiproliferative agent. In some embodiments, the one or more immunosuppressive agents is a corticosteroid.

In some embodiments, the one or more immunosuppressive agents comprise CNIs and mTOR inhibitors. In some embodiments, the one or more immunosuppressive agents comprise CNIs and antiproliferative agents. In some embodiments, the one or more immunosuppressive agents comprise CNIs and corticosteroids. In some embodiments, the one or more immunosuppressive agents comprise mTOR inhibitors and antiproliferative agents. In some embodiments, the one or more immunosuppressive agents comprise mTOR inhibitors and corticosteroids.

In some embodiments, the one or more immunosuppressive agents comprise CNIs, mTOR inhibitors, and antiproliferative agents. In some embodiments, the one or more immunosuppressive agents comprise CNIs, mTOR inhibitors, and corticosteroids. In some embodiments, the one or more immunosuppressive agents comprise CNIs, antiproliferative agents, and corticosteroids. In some embodiments, the one or more immunosuppressive agents comprise mTOR inhibitors, antiproliferative agents, and corticosteroids.

In some embodiments, the administration of the alpha-biased IL-2 variant results in a successful taper of the standard of care. In some embodiments, the administration of the alpha-biased IL-2 variant results in a successful withdrawal of the standard of care.

In some embodiments, the administration of the alpha-biased IL-2 variant results in a successful taper (reduction of dose) and/or withdrawal of the immunosuppressive agents. In some embodiments, the administration of the alpha-biased IL-2 variant results in a successful taper of the immunosuppressive agents. In some embodiments, the administration of the alpha-biased IL-2 variant results in a successful withdrawal of the immunosuppressive agents. In some embodiments, the administration of the alpha-biased IL-2 variant results in a successful taper and/or withdrawal of rapamycin. In some embodiments, the administration of the alpha-biased IL-2 variant results in a successful taper and/or withdrawal of CNI. In some embodiments, the administration of the alpha-biased IL-2 variant results in a successful taper and/or withdrawal of Tacrolimus. In some embodiments, the administration of the alpha-biased IL-2 variant results in a successful taper and/or withdrawal of mycophenolate mofetil (MMF). In some embodiments, the administration of the alpha-biased IL-2 variant results in a successful taper and/or withdrawal of corticosteroids.

In some embodiments, the alpha-biased IL-2 variant is administered prior to the kidney transplantation. In some embodiments, the alpha-biased IL-2 variant is administered at least once prior to the kidney transplantation. In some embodiments, the alpha-biased IL-2 variant is administered at least twice prior to the kidney transplantation. In some embodiments, the alpha-biased IL-2 variant is administered at least three times prior to the kidney transplantation. In some embodiments, the alpha-biased IL-2 variant is administered at least four times prior to the kidney transplantation.

In some embodiments, the alpha-biased IL-2 variant is administered subsequent to the kidney transplantation. In some embodiments, the alpha-biased IL-2 variant is administered at least once subsequent to the kidney transplantation. In some embodiments, the alpha-biased IL-2 variant is administered at least twice subsequent to the kidney transplantation. In some embodiments, the alpha-biased IL-2 variant is administered at least three times subsequent to the kidney transplantation. In some embodiments, the alpha-biased IL-2 variant is administered at least four times subsequent to the kidney transplantation. In some embodiments, the alpha-biased IL-2 variant is administered at least five times subsequent to the kidney transplantation. In some embodiments, the alpha-biased IL-2 variant is administered at least six times subsequent to the kidney transplantation. In some embodiments, the alpha-biased IL-2 variant is administered for the life of the recipient subsequent to the kidney transplantation.

In some embodiments, the alpha-biased IL-2 variant is administered both prior to kidney transplantation and subsequent to kidney transplantation. In some embodiments, the alpha-biased IL-2 variant is administered simultaneously with the kidney transplantation. In some embodiments the IL-2 variant is administered prior to, with, and subsequent to the kidney transplantation.

In one aspect, the present invention provides, among other things, a method of conditioning a kidney transplant recipient comprising administering an alpha-biased IL-2 variant to the recipient prior to the kidney transplant at a therapeutically effective amount. In some embodiments, the method of conditioning a kidney transplant recipient further comprises administration of the alpha-biased IL-2 variant subsequent to the kidney transplant.

In some embodiments, the administration of the alpha-biased IL-2 variant results in the kidney transplant to be stable for at least 20 days. In some embodiments, the administration of the alpha-biased IL-2 variant results in the kidney transplant to be stable for at least 30 days. In some embodiments, the administration of the alpha-biased IL-2 variant results in the kidney transplant to be stable for at least 10 days. In some embodiments, the administration of the alpha-biased IL-2 variant results in the kidney transplant to be stable for at least 50 days. In some embodiments, the administration of the alpha-biased IL-2 variant results in the kidney transplant to be stable for at least 60 days. In some embodiments, the administration of the alpha-biased IL-2 variant results in the kidney transplant to be stable for at least 70 days. In some embodiments, the administration of the alpha-biased IL-2 variant results in the kidney transplant to be stable for at least 80 days. In some embodiments, the administration of the alpha-biased IL-2 variant results in the kidney transplant to be stable for at least 90 days. In some embodiments, the administration of the alpha-biased IL-2 variant results in the kidney transplant to be stable for at least 100 days. In some embodiments, the administration of the alpha-biased IL-2 variant results in the kidney transplant to be stable for at least 120 days. In some embodiments, the administration of the alpha-biased IL-2 variant results in the kidney transplant to be stable for at least 140 days. In some embodiments, the administration of the alpha-biased IL-2 variant results in the kidney transplant to be stable for at least 150 days. In some embodiments, the administration of the alpha-biased IL-2 variant results in the kidney transplant to be stable for at least 160 days. In some embodiments, the administration of the alpha-biased IL-2 variant results in the kidney transplant to be stable for at least 170 days. In some embodiments, the administration of the alpha-biased IL-2 variant results in the kidney transplant to be stable for at least 180 days. In some embodiments, the administration of the alpha-biased IL-2 variant results in the kidney transplant to be stable for at least 200 days. In some embodiments, the administration of the alpha-biased IL-2 variant results in the kidney transplant to be stable for at least 225 days. In some embodiments, the administration of the alpha-biased IL-2 variant results in the kidney transplant to be stable for at least 250 days. In some embodiments, the administration of the alpha-biased IL-2 variant results in the kidney transplant to be stable for at least 275 days. In some embodiments, the administration of the alpha-biased IL-2 variant results in the kidney transplant to be stable for at least 300 days. In some embodiments, the administration of the alpha-biased IL-2 variant results in the kidney transplant to be stable for at least 325 days. In some embodiments, the administration of the alpha-biased IL-2 variant results in the kidney transplant to be stable for at least 350 days.

In some embodiments, the administration of the alpha-biased IL-2 variant results in the kidney transplant to be stable for at least 3 months. In some embodiments, the administration of the alpha-biased IL-2 variant results in the kidney transplant to be stable for at least 4 months. In some embodiments, the administration of the alpha-biased IL-2 variant results in the kidney transplant to be stable for at least 5 months. In some embodiments, the administration of the alpha-biased IL-2 variant results in the kidney transplant to be stable for at least 6 months. In some embodiments, the administration of the alpha-biased IL-2 variant results in the kidney transplant to be stable for at least 8 months. In some embodiments, the administration of the alpha-biased IL-2 variant results in the kidney transplant to be stable for at least 10 months. In some embodiments, the administration of the alpha-biased IL-2 variant results in the kidney transplant to be stable for at least 12 months. In some embodiments, the administration of the alpha-biased IL-2 variant results in the kidney transplant to be stable for at least 14 months. In some embodiments, the administration of the alpha-biased IL-2 variant results in the kidney transplant to be stable for at least 16 months. In some embodiments, the administration of the alpha-biased IL-2 variant results in the kidney transplant to be stable for at least 18 months. In some embodiments, the administration of the alpha-biased IL-2 variant results in the kidney transplant to be stable for at least 20 months. In some embodiments, the administration of the alpha-biased IL-2 variant results in the kidney transplant to be stable for at least 24 months.

In some embodiments, the administration of the alpha-biased IL-2 variant results in the kidney transplant to be stable for at least 1 year. In some embodiments, the administration of the alpha-biased IL-2 variant results in the kidney transplant to be stable for at least 2 years. In some embodiments, the administration of the alpha-biased IL-2 variant results in the kidney transplant to be stable for at least 3 years. In some embodiments, the administration of the alpha-biased IL-2 variant results in the kidney transplant to be stable for at least 4 years. In some embodiments, the administration of the alpha-biased IL-2 variant results in the kidney transplant to be stable for at least 5 years. In some embodiments, the administration of the alpha-biased IL-2 variant results in the kidney transplant to be stable for the life of the recipient.

In some embodiments, the alpha-biased IL-2 variant is administered to the recipient at least once, at least twice, at least three times, or at least four times prior to the kidney transplantation. In some embodiments, the alpha-biased IL-2 variant is administered to the recipient at least once prior to the kidney transplantation. In some embodiments, the alpha-biased IL-2 variant is administered to the recipient at least twice prior to the kidney transplantation. In some embodiments, the alpha-biased IL-2 variant is administered to the recipient at least three times prior to the kidney transplantation. In some embodiments, the alpha-biased IL-2 variant is administered to the recipient at least four times prior to the kidney transplantation.

In some embodiments, the alpha-biased IL-2 variant is administered to the recipient at least once, at least twice, at least three times, at least four times, at least five times, or at least six times subsequent to the kidney transplantation. In some embodiments, the alpha-biased IL-2 variant is administered to the recipient at least once subsequent to the kidney transplantation. In some embodiments, the alpha-biased IL-2 variant is administered to the recipient at least twice subsequent to the kidney transplantation. In some embodiments, the alpha-biased IL-2 variant is administered to the recipient at least three times subsequent to the kidney transplantation. In some embodiments, the alpha-biased IL-2 variant is administered to the recipient at least four times subsequent to the kidney transplantation. In some embodiments, the alpha-biased IL-2 variant is administered to the recipient at least five times subsequent to the kidney transplantation. In some embodiments, the alpha-biased IL-2 variant is administered to the recipient at least six times subsequent to the kidney transplantation. In some embodiments, the alpha-biased IL-2 variant is administered to the recipient for the lifetime subsequent to the kidney transplantation.

In some embodiments, the method described herein further comprises discontinuing the administration of the alpha-biased IL-2 variant. In some embodiments, the method comprises discontinuing the administration of the alpha-biased IL-2 variant within 30 days of the kidney transplantation. In some embodiments, the method comprises discontinuing the administration of the alpha-biased IL-2 variant within 45 days of the kidney transplantation. In some embodiments, the method comprises discontinuing the administration of the alpha-biased IL-2 variant within 60 days of the kidney transplantation. In some embodiments, the method comprises discontinuing the administration of the alpha-biased IL-2 variant within 75 days of the kidney transplantation. In some embodiments, the method comprises discontinuing the administration of the alpha-biased IL-2 variant within 90 days of the kidney transplantation. In some embodiments, the method comprises discontinuing the administration of the alpha-biased IL-2 variant within 105 days of the kidney transplantation. In some embodiments, the method comprises discontinuing the administration of the alpha-biased IL-2 variant within 120 days of the kidney transplantation. In some embodiments, the method comprises discontinuing the administration of the alpha-biased IL-2 variant within 135 days of the kidney transplantation. In some embodiments, the method comprises discontinuing the administration of the alpha-biased IL-2 variant within 150 days of the kidney transplantation. In some embodiments, the method comprises discontinuing the administration of the alpha-biased IL-2 variant within 165 days of the kidney transplantation. In some embodiments, the method comprises discontinuing the administration of the alpha-biased IL-2 variant within 180 days of the kidney transplantation.

In some embodiments, the alpha-biased IL-2 variant is engineered to have increased affinity to the IL-2 alpha-receptor CD25. In some embodiments, the alpha-biased IL-2 variant is engineered to have decreased affinity to the IL-2 beta and/or gamma receptors CD122 and/or CD132. In some embodiments, the alpha-biased IL-2 variant is engineered to have preferential binding to the trimeric IL-2 receptor (alpha, beta, and gamma) over the dimeric IL-2 receptor (beta and gamma). In some embodiments, the alpha-biased IL-2 variant is further engineered for increased protein stability. In some embodiments, the alpha-biased IL-2 variant is further engineered for increased protein expression.

In some embodiments, the alpha-biased IL-2 variant comprises an amino acid substitution of H16L, H16N, V69A, Q74P, I92S, D84V, and/or S87R. In some embodiments, the alpha-biased IL-2 variant comprises an amino acid substitution of H16L. In some embodiments, the alpha-biased IL-2 variant comprises an amino acid substitution of H16N. In some embodiments, the alpha-biased IL-2 variant comprises an amino acid substitution of V69A. In some embodiments, the alpha-biased IL-2 variant comprises an amino acid substitution of Q74P. In some embodiments, the alpha-biased IL-2 variant comprises an amino acid substitution of I92S. In some embodiments, the alpha-biased IL-2 variant comprises an amino acid substitution of D84V. In some embodiments, the alpha-biased IL-2 variant comprises an amino acid substitution of S87R.

In some embodiments, the alpha-biased IL-2 variant comprises the amino acid substitutions of H16L, V69A, and Q74P. In some embodiments, the alpha-biased IL-2 variant comprises the amino acid substitutions of H16N, V69A, and Q74P. In some embodiments, the alpha-biased IL-2 variant comprises the amino acid substitutions of V69A, Q74P, and S87R. In some embodiments, the alpha-biased IL-2 variant comprises the amino acid substitutions of V69A, Q74P, and D84V. In some embodiments, the alpha-biased IL-2 variant comprises the amino acid substitutions of V69A, Q74P, and I92S.

In some embodiments, the alpha-biased IL-2 variant further comprises the amino acid substitution of C125S. In some embodiments, the alpha-biased IL-2 variant further comprises the amino acid substitution of C125A.

In some embodiments, the alpha-biased IL-2 variant comprises an amino acid sequence that is at least 90%, at least 95%, at least 98%, or at least 99% identical to SEQ ID NO: 4. In some embodiments, the alpha-biased IL-2 variant comprises an amino acid sequence that is at least 90% identical to SEQ ID NO: 4. In some embodiments, the alpha-biased IL-2 variant comprises an amino acid sequence that is at least 95% identical to SEQ ID NO: 4. In some embodiments, the alpha-biased IL-2 variant comprises an amino acid sequence that is at least 98% identical to SEQ ID NO: 4. In some embodiments, the alpha-biased IL-2 variant comprises an amino acid sequence that is at least 99% identical to SEQ ID NO: 4. In some embodiments, the alpha-biased IL-2 variant comprises an amino acid sequence that is identical to SEQ ID NO: 4.

In some embodiments, the alpha-biased IL-2 variant comprises the amino acid substitutions of H16L, V69A, and Q74P, and comprises an amino acid sequence at least 95% identical to SEQ ID NO: 3. In some embodiments, the alpha-biased IL-2 variant comprises the amino acid substitutions of H16N, V69A, and Q74P, and comprises an amino acid sequence at least 95% identical to SEQ ID NO: 3. In some embodiments, the alpha-biased IL-2 variant comprises the amino acid substitutions of V69A, Q74P, and D84V, and comprises an amino acid sequence at least 95% identical to SEQ ID NO: 3. In some embodiments, the alpha-biased IL-2 variant comprises the amino acid substitutions of V69A, Q74P, and S87R, and comprises an amino acid sequence at least 95% identical to SEQ ID NO: 3. In some embodiments, the alpha-biased IL-2 variant comprises the amino acid substitutions of V69A, Q74P, and I92S, and comprises an amino acid sequence at least 95% identical to SEQ ID NO: 3.

In some embodiments, the alpha-biased IL-2 variant is fused to a half-life extension domain. In some embodiments, the half-life extension domain is an Fc domain, a human serum albumin, or an albumin binding domain. In some embodiments, the half-life extension domain is an Fc domain. In some embodiments, the half-life extension domain is a human serum albumin. In some embodiments, the half-life extension domain is an albumin binding domain.

In some embodiments, the Fc domain comprises an IgG1 Fc domain, an IgG2 Fc domain, an IgG3 Fc domain, or an IgG4 Fc domain. In some embodiments, the Fc domain comprises an IgG1 Fc domain. In some embodiments, the Fc domain comprises an IgG2 Fc domain. In some embodiments, the Fc domain comprises an IgG3 Fc domain. In some embodiments, the Fc domain comprises an IgG4 Fc domain.

In some embodiments, the Fc domain is engineered to reduce effector function. In some embodiments, the Fc domain is engineered to further increase half-life. In some embodiments, the Fc domain comprises an amino acid substitution of N297G.

In some embodiments, the alpha-biased IL-2 variant is fused to the half-life extension domain via a linker. In some embodiments, the linker is a Gly-Ser linker. In some embodiments, the linker comprises (G4S)ₙ, wherein n is 1, 2, 3, 4, 5, 6, or more. In some embodiments, the linker comprises (G4S)₁ (SEQ ID NO: 14). In some embodiments, the linker comprises (G4S)₂. In some embodiments, the linker comprises (G4S)₃. In some embodiments, the linker comprises (G4S)₄. In some embodiments, the linker comprises (G4S)₅. In some embodiments, the linker comprises (G4S)₆.

In some embodiments, the alpha-biased IL-2 variant fused to the Fc domain comprises an amino acid sequence that is at least 90%, at least 95%, at least 98%, or at least 99% identical to SEQ ID NO: 27. In some embodiments, the alpha-biased IL-2 variant fused to the Fc domain comprises an amino acid sequence that is at least 90% identical to SEQ ID NO: 27. In some embodiments, the alpha-biased IL-2 variant fused to the Fc domain comprises an amino acid sequence that is at least 95% identical to SEQ ID NO: 27. In some embodiments, the alpha-biased IL-2 variant fused to the Fc domain comprises an amino acid sequence that is at least 98% identical to SEQ ID NO: 27. In some embodiments, the alpha-biased IL-2 variant fused to the Fc domain comprises an amino acid sequence that is at least 99% identical to SEQ ID NO: 27. In some embodiments, the alpha-biased IL-2 variant fused to the Fc domain comprises an amino acid sequence that is identical to SEQ ID NO: 27.

In some embodiments, the alpha-biased IL-2 variant comprises the amino acid substitutions of H16L, V69A, and Q74P, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of H16N, V69A, and Q74P, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and D84V, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and S87R, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and I92S, and the alpha-biased IL-2 variant fused to the Fc domain comprises an amino acid sequence at least 95% identical to SEQ ID NO: 26.

In some embodiments, the methods described herein further comprises administering an immunosuppressive agent. In some embodiments, the immunosuppressive agent is administered prior to kidney transplantation. In some embodiments, the immunosuppressive agent is administered subsequent to kidney transplantation. In some embodiments, the immunosuppressive agent is administered prior to and subsequent to kidney transplantation.

In some embodiments, the immunosuppressive agent is administered parenterally. In some embodiments, the immunosuppressive agent is administered intravenously. In some embodiments, the immunosuppressive agent is administered intramuscularly. In some embodiments, the immunosuppressive agent is administered subcutaneously.

In some embodiments, administration of the immunosuppressive agent is discontinued within 180 days of the kidney transplantation. In some embodiments, administration of the immunosuppressive agent is discontinued within 165 days of the kidney transplantation. In some embodiments, administration of the immunosuppressive agent is discontinued within 150 days of the kidney transplantation. In some embodiments, administration of the immunosuppressive agent is discontinued within 135 days of the kidney transplantation. In some embodiments, administration of the immunosuppressive agent is discontinued within 120 days of the kidney transplantation. In some embodiments, administration of the immunosuppressive agent is discontinued within 105 days of the kidney transplantation. In some embodiments, administration of the immunosuppressive agent is discontinued within 90 days of the kidney transplantation. In some embodiments, administration of the immunosuppressive agent is discontinued within 75 days of the kidney transplantation. In some embodiments, administration of the immunosuppressive agent is discontinued within 60 days of the kidney transplantation. In some embodiments, administration of the immunosuppressive agent is discontinued within 45 days of the kidney transplantation. In some embodiments, administration of the immunosuppressive agent is discontinued within 30 days of the kidney transplantation.

In some embodiments, the methods described herein further comprises providing a standard of care.

In some embodiments, the standard of care is discontinued within 180 days of the kidney transplantation. In some embodiments, the standard of care is discontinued within 165 days of the kidney transplantation. In some embodiments, the standard of care is discontinued within 150 days of the kidney transplantation. In some embodiments, the standard of care is discontinued within 135 days of the kidney transplantation. In some embodiments, the standard of care is discontinued within 120 days of the kidney transplantation. In some embodiments, the standard of care is discontinued within 105 days of the kidney transplantation. In some embodiments, the standard of care is discontinued within 90 days of the kidney transplantation. In some embodiments, the standard of care is discontinued within 75 days of the kidney transplantation. In some embodiments, the standard of care is discontinued within 60 days of the kidney transplantation. In some embodiments, the standard of care is discontinued within 45 days of the kidney transplantation. In some embodiments, the standard of care is discontinued within 30 days of the kidney transplantation.

In some embodiments, the recipient has a creatinine level below 4 mg/dL for at least one week, at least two weeks, at least three weeks, at least one month, at least two months, at least three months, or at least four months. In some embodiments, the recipient has a creatinine level below 4 mg/dL for at least one week. In some embodiments, the recipient has a creatinine level below 4 mg/dL for at least two weeks. In some embodiments, the recipient has a creatinine level below 4 mg/dL for at least three weeks. In some embodiments, the recipient has a creatinine level below 4 mg/dL for at least one month. In some embodiments, the recipient has a creatinine level below 4 mg/dL for at least two months. In some embodiments, the recipient has a creatinine level below 4 mg/dL for at least three months. In some embodiments, the recipient has a creatinine level below 4 mg/dL for at least four months. In some embodiments, the recipient has a creatinine level below 4 mg/dL for at least five months. In some embodiments, the recipient has a creatinine level below 4 mg/dL for at least six months. In some embodiments, the recipient has a creatinine level below 4 mg/dL for at least seven months. In some embodiments, the recipient has a creatinine level below 4 mg/dL for at least eight months. In some embodiments, the recipient has a creatinine level below 4 mg/dL for at least ten months. In some embodiments, the recipient has a creatinine level below 4 mg/dL for at least twelve months. In some embodiments, the recipient has a creatinine level below 4 mg/dL for at least fourteen months. In some embodiments, the recipient has a creatinine level below 4 mg/dL for at least sixteen months. In some embodiments, the recipient has a creatinine level below 4 mg/dL for at least eighteen months. In some embodiments, the recipient has a creatinine level below 4 mg/dL for at least twenty months. In some embodiments, the recipient has a creatinine level below 4 mg/dL for at least twenty-four months. In some embodiments, the recipient has a creatinine level below 4 mg/dL for at least thirty months. In some embodiments, the recipient has a creatinine level below 4 mg/dL for at least thirty-six months.

In some embodiments, the recipient has a blood urea nitrogen (BUN) level below 50 mg/dL for at least one week, at least two weeks, at least three weeks, at least one month, at least two months, at least three months, or at least four months. In some embodiments, the recipient has a BUN level below 50 mg/dL for at least one week. In some embodiments, the recipient has a BUN level below 50 mg/dL for at least two weeks. In some embodiments, the recipient has a BUN level below 50 mg/dL for at least three weeks. In some embodiments, the recipient has a BUN level below 50 mg/dL for at least one month. In some embodiments, the recipient has a BUN level below 50 mg/dL for at least two months. In some embodiments, the recipient has a BUN level below 50 mg/dL for at least three months. In some embodiments, the recipient has a BUN level below 50 mg/dL for at least four months. In some embodiments, the recipient has a BUN level below 50 mg/dL for at least five months. In some embodiments, the recipient has a BUN level below 50 mg/dL for at least six months. In some embodiments, the recipient has a BUN level below 50 mg/dL for at least seven months. In some embodiments, the recipient has a BUN level below 50 mg/dL for at least eight months. In some embodiments, the recipient has a BUN level below 50 mg/dL for at least ten months. In some embodiments, the recipient has a BUN level below 50 mg/dL for at least twelve months. In some embodiments, the recipient has a BUN level below 50 mg/dL for at least fourteen months. In some embodiments, the recipient has a BUN level below 50 mg/dL for at least sixteen months. In some embodiments, the recipient has a BUN level below 50 mg/dL for at least eighteen months. In some embodiments, the recipient has a BUN level below 50 mg/dL for at least twenty months. In some embodiments, the recipient has a BUN level below 50 mg/dL for at least twenty-four months. In some embodiments, the recipient has a BUN level below 50 mg/dL for at least thirty months. In some embodiments, the recipient has a BUN level below 50 mg/dL for at least thirty-six months.

In some embodiments, the percent of CD4⁺CD25⁺FoxP3⁺ Treg cells in CD4⁺ T cells in a population of CD4⁺ T cells in a recipient is increased after administration of the alpha-biased IL-2 variant. In some embodiments, the number of cytotoxic T lymphocytes is not substantially increased after administration of the alpha-biased IL-2 variant.

In one aspect, the present invention provides, among other things, a method for prophylaxis for kidney rejection in a recipient receiving a kidney transplant comprising administering to the recipient an IL-2 variant in conjunction with the kidney transplant, wherein the IL-2 variant comprises one or more amino acid substitutions selected from Table 1. In some embodiments, the one or more amino acid substitutions are selected from the group consisting of H16L, H16N, V69A, Q74P, I92S, D84V, and S87R.

In some embodiments, the IL-2 variant comprises an amino acid substitution of H16L. In some embodiments, the IL-2 variant comprises an amino acid substitution of H16N. In some embodiments, the IL-2 variant comprises an amino acid substitution of V69A. In some embodiments, the IL-2 variant comprises an amino acid substitution of Q74P. In some embodiments, the IL-2 variant comprises an amino acid substitution of I92S. In some embodiments, the IL-2 variant comprises an amino acid substitution of D84V. In some embodiments, the IL-2 variant comprises an amino acid substitution of S87R.

In some embodiments, the IL-2 variant comprises the amino acid substitutions of H16L, V69A, and Q74P. In some embodiments, the IL-2 variant comprises the amino acid substitutions of H16N, V69A, and Q74P. In some embodiments, the IL-2 variant comprises the amino acid substitutions of V69A, Q74P, and S87R. In some embodiments, the IL-2 variant comprises the amino acid substitutions of V69A, Q74P, and D84V. In some embodiments, the IL-2 variant comprises the amino acid substitutions of V69A, Q74P, and I92S.

In some embodiments, the IL-2 variant further comprises the amino acid substitution of C125S.

In some embodiments, the IL-2 variant comprises an amino acid sequence that is at least 90%, at least 95%, at least 98%, or at least 99% identical to SEQ ID NO: 4. In some embodiments, the IL-2 variant comprises an amino acid sequence that is at least 90% identical to SEQ ID NO: 4. In some embodiments, the IL-2 variant comprises an amino acid sequence that is at least 95% identical to SEQ ID NO: 4. In some embodiments, the IL-2 variant comprises an amino acid sequence that is at least 98% identical to SEQ ID NO: 4. In some embodiments, the IL-2 variant comprises an amino acid sequence that is at least 99% identical to SEQ ID NO: 4. In some embodiments, the IL-2 variant is identical to SEQ ID NO: 4.

In some embodiments, the IL-2 variant comprises the amino acid substitutions of H16L, V69A, and Q74P, and comprises an amino acid sequence at least 95% identical to SEQ ID NO: 3. In some embodiments, the IL-2 variant comprises the amino acid substitutions of H16N, V69A, and Q74P, and comprises an amino acid sequence at least 95% identical to SEQ ID NO: 3. In some embodiments, the IL-2 variant comprises the amino acid substitutions of V69A, Q74P, and D84V, and comprises an amino acid sequence at least 95% identical to SEQ ID NO: 3. In some embodiments, the IL-2 variant comprises the amino acid substitutions of V69A, Q74P, and S87R, and comprises an amino acid sequence at least 95% identical to SEQ ID NO: 3. In some embodiments, the IL-2 variant comprises the amino acid substitutions of V69A, Q74P, and I92S, and comprises an amino acid sequence at least 95% identical to SEQ ID NO: 3.

In some embodiments, the IL-2 variant is fused to a half-life extension domain. In some embodiments, the half-life extension domain is an Fc domain, a human serum albumin, or an albumin binding domain. In some embodiments, the half-life extension domain is an Fc domain. In some embodiments, the half-life extension domain is a human serum albumin. In some embodiments, the half-life extension domain is an albumin binding domain.

In some embodiments, the Fc domain comprises an IgG1 Fc domain, an IgG2 Fc domain, an IgG3 Fc domain, or an IgG4 Fc domain. In some embodiments, the Fc domain comprises an IgG1 Fc domain. In some embodiments, the Fc domain comprises an IgG2 Fc domain. In some embodiments, the Fc domain comprises an IgG3 Fc domain. In some embodiments, the Fc domain comprises an IgG4 Fc domain.

In some embodiments, the Fc domain is engineered to reduce effector function. In some embodiments, the Fc domain is engineered to further increase half-life. In some embodiments, the Fc domain comprises an amino acid substitution of N297G.

In some embodiments, the IL-2 variant is fused to the half-life extension domain via a linker. In some embodiments, the linker is a Gly-Ser linker. In some embodiments, the linker comprises (G4S)ₙ, wherein n is 1, 2, 3, 4, 5, 6, or more. In some embodiments, the linker comprises (G4S)₁. In some embodiments, the linker comprises (G4S)₂. In some embodiments, the linker comprises (G4S)₃. In some embodiments, the linker comprises (G4S)₄. In some embodiments, the linker comprises (G4S)₅. In some embodiments, the linker comprises (G4S)₆.

In some embodiments, the IL-2 variant fused to the Fc domain comprises an amino acid sequence that is at least 90%, at least 95%, at least 98%, or at least 99% identical to SEQ ID NO: 27. In some embodiments, the IL-2 variant fused to the Fc domain comprises an amino acid sequence that is at least 90% identical to SEQ ID NO: 27. In some embodiments, the IL-2 variant fused to the Fc domain comprises an amino acid sequence that is at least 92% identical to SEQ ID NO: 27. In some embodiments, the IL-2 variant fused to the Fc domain comprises an amino acid sequence that is at least 93% identical to SEQ ID NO: 27. In some embodiments, the IL-2 variant fused to the Fc domain comprises an amino acid sequence that is at least 94% identical to SEQ ID NO: 27. In some embodiments, the IL-2 variant fused to the Fc domain comprises an amino acid sequence that is at least 95% identical to SEQ ID NO: 27. In some embodiments, the IL-2 variant fused to the Fc domain comprises an amino acid sequence that is at least 96% identical to SEQ ID NO: 27. In some embodiments, the IL-2 variant fused to the Fc domain comprises an amino acid sequence that is at least 97% identical to SEQ ID NO: 27. In some embodiments, the IL-2 variant fused to the Fc domain comprises an amino acid sequence that is at least 98% identical to SEQ ID NO: 27. In some embodiments, the IL-2 variant fused to the Fc domain comprises an amino acid sequence that is at least 99% identical to SEQ ID NO: 27. In some embodiments, the IL-2 variant fused to the Fc domain comprises an amino acid sequence that is identical to SEQ ID NO: 27.

In some embodiments, the IL-2 variant comprises the amino acid substitutions of H16L, V69A, and Q74P, and the IL-2 variant fused to the Fc domain comprises an amino acid sequence at least 95% identical to SEQ ID NO: 26. In some embodiments, the IL-2 variant comprises the amino acid substitutions of H16N, V69A, and Q74P, and the IL-2 variant fused to the Fc domain comprises an amino acid sequence at least 95% identical to SEQ ID NO: 26. In some embodiments, the IL-2 variant comprises the amino acid substitutions of V69A, Q74P, and D84V, and the IL-2 variant fused to the Fc domain comprises an amino acid sequence at least 95% identical to SEQ ID NO: 26. In some embodiments, the IL-2 variant comprises the amino acid substitutions of V69A, Q74P, and S87R, and the IL-2 variant fused to the Fc domain comprises an amino acid sequence at least 95% identical to SEQ ID NO: 26. In some embodiments, the IL-2 variant comprises the amino acid substitutions of V69A, Q74P, and I92S, and the IL-2 variant fused to the Fc domain comprises an amino acid sequence at least 95% identical to SEQ ID NO: 26.

In some embodiments, the IL-2 variant fused to the Fc domain comprises an amino acid sequence that is at least 90%, at least 95%, at least 98%, or at least 99% identical to SEQ ID NO: 26. In some embodiments, the IL-2 variant fused to the Fc domain comprises an amino acid sequence that is at least 90% identical to SEQ ID NO: 26. In some embodiments, the IL-2 variant fused to the Fc domain comprises an amino acid sequence that is at least 92% identical to SEQ ID NO: 26. In some embodiments, the IL-2 variant fused to the Fc domain comprises an amino acid sequence that is at least 93% identical to SEQ ID NO: 26. In some embodiments, the IL-2 variant fused to the Fc domain comprises an amino acid sequence that is at least 94% identical to SEQ ID NO: 26. In some embodiments, the IL-2 variant fused to the Fc domain comprises an amino acid sequence that is at least 95% identical to SEQ ID NO: 26. In some embodiments, the IL-2 variant fused to the Fc domain comprises an amino acid sequence that is at least 96% identical to SEQ ID NO: 26. In some embodiments, the IL-2 variant fused to the Fc domain comprises an amino acid sequence that is at least 97% identical to SEQ ID NO: 26. In some embodiments, the IL-2 variant fused to the Fc domain comprises an amino acid sequence that is at least 98% identical to SEQ ID NO: 26. In some embodiments, the IL-2 variant fused to the Fc domain comprises an amino acid sequence that is at least 99% identical to SEQ ID NO: 26. In some embodiments, the IL-2 variant fused to the Fc domain comprises an amino acid sequence that is identical to SEQ ID NO: 26.

In some embodiments, administration of the IL-2 variant improves the kidney transplant in the recipient as compared to a control. In some embodiments, the improvement in the kidney transplant results in prolonged stability of the kidney transplant, survival of the recipient, and reduced rejection symptoms as compared to a control. In some embodiments, the improvement in the kidney transplant results in prolonged stability of the kidney transplant, survival of the recipient, or reduced rejection symptoms as compared to a control. In some embodiments, the improvement in the kidney transplant results in prolonged stability of the kidney transplant. In some embodiments, the improvement in the kidney transplant results in survival of the recipient. In some embodiments, the improvement in the kidney transplant results in reduced rejection symptoms as compared to a control. In some embodiments, the improvement in the kidney transplant results in prolonged stability of the kidney transplant and reduced rejection symptoms as compared to a control. In some embodiments, the improvement in the kidney transplant results in survival of the recipient and reduced rejection symptoms as compared to a control. In some embodiments, the improvement in the kidney transplant results in prolonged stability of the kidney transplant and survival of the recipient.

In some embodiments, the control is historical data. In some embodiments, the control is a comparable recipient without the administration of the IL-2 variant. In some embodiments, the control is a comparable recipient receiving a standard of care. In some embodiments, the control is a comparable recipient receiving a standard of care without the administration of the IL-2 variant.

In some embodiments, the IL-2 variant is administered prior to kidney transplantation. In some embodiments, the IL-2 variant is administered subsequent to kidney transplantation. In some embodiments, the IL-2 variant is administered prior to and subsequent to kidney transplantation. In some embodiments, the IL-2 variant is administered simultaneously with the kidney transplantation.

In some embodiments, the IL-2 variant is administered at least once, at least twice, at least three times, or at least four times prior to the kidney transplantation. In some embodiments, the IL-2 variant is administered at least once prior to the kidney transplantation. In some embodiments, the IL-2 variant is administered at least twice prior to the kidney transplantation. In some embodiments, the IL-2 variant is administered at least three times prior to the kidney transplantation. In some embodiments, the IL-2 variant is administered at least four times prior to the kidney transplantation.

In some embodiments, the IL-2 variant is administered at least once, at least twice, at least three times, at least four times, at least five times, or at least six times subsequent to the kidney transplantation. In some embodiments, the IL-2 variant is administered at least once subsequent to the kidney transplantation. In some embodiments, the IL-2 variant is administered at least twice subsequent to the kidney transplantation. In some embodiments, the IL-2 variant is administered at least three times subsequent to the kidney transplantation. In some embodiments, the IL-2 variant is administered at least four times subsequent to the kidney transplantation. In some embodiments, the IL-2 variant is administered at least five times subsequent to the kidney transplantation. In some embodiments, the IL-2 variant is administered at least six times subsequent to the kidney transplantation. In some embodiments, the IL-2 variant is administered for the life of the recipient subsequent to the kidney transplantation.

### BRIEF DESCRIPTION OF DRAWINGS

FIG. 1A is an exemplary schematic that outlines the study design to assess the immunomodulatory effect of an exemplary alpha-biased IL-2-Fc fusion protein in nonhuman primates (NHP). The arrows in FIG. 1A indicate the administration of the alpha-biased IL-2-Fc fusion protein to NHP. FIG. 1B and FIG. 1C are exemplary schematics outlining study designs to assess kidney allograft survival in NHPs after administration of an exemplary alpha-biased IL-2-Fc fusion protein. The exemplary study designs also include 6 months (FIG. 1B) or 1 month (FIG. 1C) of daily rapamycin administration, as illustrated in Examples 2 and 3, respectively.

FIG. 2A is an exemplary plot showing the percent of Treg cells (CD4⁺CD25⁺FoxP3⁺) both pre- and post-treatment with the exemplary alpha-biased IL-2-Fc fusion protein. FIG. 2B is an exemplary graph showing the increase in absolute Treg cell count as well as the increase in the ratio of Tregs:CD8 cells after treatment with the alpha-biased IL-2-Fc fusion protein. The increase in Treg cells was both statistically significant and dose dependent.

FIG. 3A is a series of exemplary graph showing the increase in the absolute number of Tregs over the course of treatment and after each dose of the alpha-biased IL-2-Fc fusion protein. FIG. 3B is a series of exemplary graphs showing the increase in the percent of Tregs over the course of treatment and after each dose. As shown in FIG. 1A, the alpha-biased IL-2-Fc fusion protein was administered at day 0, 14, and 20. “Pre” refers to the absolute number or percent, respectively, prior to administration of the alpha-biased IL-2-Fc fusion protein, and “post” refers to the absolute number or percent, respectively, after administration of the alpha-biased IL-2-Fc fusion protein.

FIG. 4A and FIG. 4B are exemplary graphs showing key measures of kidney function after kidney transplant and administration of an exemplary alpha-biased IL-2-Fc fusion protein. FIG. 4A shows the creatinine levels, and FIG. 4B shows the blood urea nitrogen (BUN) levels.

FIG. 5 is a series of exemplary graphs showing the percent of CD20⁺ B cells out of the total population of lymphocytes as well as the absolute number of CD20⁺ B cells after kidney transplant and administration of an exemplary alpha-biased IL-2-Fc fusion protein.

FIG. 6 is a series of exemplary graphs showing the percent and absolute values for CD3⁺ T cells after kidney transplant and administration of an exemplary alpha-biased IL-2-Fc fusion protein.

FIG. 7 is a series of exemplary graphs showing the percent of CD4⁺ and CD8⁺ T cells out of the total population of CD3⁺ T cells as well as the absolute number of CD4⁺ and CD8⁺ T cells peripheral blood mononuclear cells (PBMCs) after kidney transplant and administration of an exemplary alpha-biased IL-2-Fc fusion protein.

FIG. 8 is a series of exemplary graphs showing the percent of FoxP3 and CD127^{lo} T cells out of the total population of CD4⁺ T cells as well as the absolute number of FoxP3 and CD127^{lo} T cells in peripheral blood mononuclear cells (PBMCs) after kidney transplant and administration of an exemplary alpha-biased IL-2-Fc fusion protein.

FIG. 9 is a series of exemplary graphs summarizing the post-transplant survival after treatment with an exemplary alpha-biased IL-2-Fc fusion protein and daily rapamycin or daily rapamycin alone. Rapamycin administration was discontinued on Day 180.

FIG. 10A and FIG. 10B are a series of exemplary graphs showing key measures of kidney function after kidney transplant and administration of an exemplary alpha-biased IL-2-Fc fusion protein. FIG. 10A shows the creatinine (sCR) and blood urea nitrogen (BUN) levels in monkeys that were administered an exemplary alpha-biased IL-2-Fc fusion protein and rapamycin. FIG. 10B shows the same in monkeys that were administered rapamycin alone.

FIG. 11 is a series of exemplary graphs showing the percent of CD4⁺ and CD8⁺ T cells out of the total population of CD3⁺ T cells as well as the absolute number of CD4⁺ and CD8⁺ T cells in PBMCs after kidney transplant and administration of an exemplary alpha-biased IL-2-Fc fusion protein.

FIG. 12 is a series of exemplary graphs summarizing the post-transplant survival after treatment with an exemplary alpha-biased IL-2-Fc fusion protein and daily rapamycin or daily rapamycin alone. Rapamycin administration was discontinued on Day 30.

### DEFINITIONS

In order for the present disclosure to be more readily understood, certain terms are first defined below. Additional definitions for the following terms and other terms are set forth throughout the specification. The publications and other reference materials referenced herein to describe the background of the disclosure and to provide additional detail regarding its practice are hereby incorporated by reference.

“Alpha-biased IL-2 variant” is used herein to refer to an IL-2 variant that comprises one or more mutations that increase binding to an IL-2 receptor alpha subunit (CD25) or decrease binding to an IL-2 receptor beta subunit (CD122) or gamma subunit (CD132) as compared to a wild-type IL-2. In some embodiments, an alpha-biased IL-2 variant has a preferential binding to a trimeric IL-2 receptor over the dimeric IL-2 receptor.

“Conditioning” is used herein to refer to a method of preparing a recipient for a kidney transplant such that the recipient is more likely to have positive transplant outcomes. In some embodiments, “conditioning” a recipient for kidney transplant prevents kidney transplant rejection, stabilizes the transplanted kidney, and/or increase long-term survival. In some embodiments, conditioning a recipient comprises administering an IL-2 variant prior to a kidney transplant. In some embodiments, conditioning a recipient comprises administering an IL-2 variant subsequent to a kidney transplant. In some embodiments, conditioning a recipient comprises administering an IL-2 variant simultaneously with a kidney transplant.

The term “Fc” refers to a portion of a heavy chain constant region that comprises at least the CH2 and CH3 domains that typically bind to an Fc receptor e.g., an FcγR, namely FcγRI (CD64), FcγRII (CD32), FcγRIII (CD16) or an FcRn, i.e., a neonatal Fc receptor. The Fc variants of the present invention may be optimized for a variety of properties. An Fc variant that is engineered or predicted to display one or more optimized properties is herein referred to as an “optimized Fc variant”. In some embodiments, an optimized Fc variant has reduced or ablated affinity for FcγRI, FcγRIIa, FcγRIIb, FcγRIIIa, FcγRIIIb, and C1q, and retains binding to FcRn.

As used herein, “in conjunction with” means “in combination with” or “together with”. When an IL-2 variant is administered “in conjunction with” a kidney transplant or a second therapeutic agent, the IL-2 variant and the kidney transplant or the second therapeutic agent can be administered in any order. In some embodiments, “in conjunction with” refers to “prior to,” “subsequent to,” or “simultaneously with.” For example, when an IL-2 variant is administered in conjunction with a kidney transplant, the IL-2 variant can be administered prior to and/or subsequent to the kidney transplant. In some embodiments, IL-2 variant is administered in conjunction with a kidney transplant such that the IL-2 variant is administered simultaneously with the kidney transplant.

As used herein, “increased half-life” or “increase serum half-life” or “extending half-life” means the positive change in the circulating half-life of a modified biologically active molecule (e.g., an alpha-biased IL-2 variant) relative to its non-modified form (or naked form of the peptide). Serum half-life is measured, for example, by taking blood samples at various time points after administration of the biologically active molecule and determining the concentration of that molecule in each sample.

As used herein “pharmaceutically acceptable” refers to being generally recognized for use in animals, and more particularly in humans.

As used herein “pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to a diluent, adjuvant, excipient, or carrier with which at least one compound of the present disclosure is administered.

As used herein “prophylaxis,” “prophylactic,” or its grammatical equivalents refer to measures designed to prevent a disease or transplant rejection. In some embodiments, “prophylaxis” comprises a preventative treatment. In some embodiments, “prophylaxis” refers to treating a transplant rejection.

As used herein, “recipient” refers to the subject who will receive, is receiving, or has received an organ transplant. The terms “recipient” and “subject” are used interchangeably herein.

As used herein, “subject” includes mammals and humans. The terms “human” and “subject” are used interchangeably herein.

As used herein, “therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Transplant” is used herein to describe both the act of transplanting an organ and the transplanted organ. In some embodiments, a kidney transplant is the physical kidney that is transplanted into the recipient. In some embodiments, a kidney transplant is the act of transplanting a donor kidney into a recipient, a process which is also known as a kidney transplantation.

As used herein, “treating” or “treatment” of kidney rejection refers to prophylaxis for kidney rejection, arresting or ameliorating a kidney rejection or its symptoms, reducing the risk of acquiring a kidney rejection or its symptoms, or reducing the development of a kidney rejection or its symptoms. “Treating” or “treatment” also refers to inhibiting the kidney rejection, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the kidney rejection, or at least symptoms thereof in a subject.

As used herein, “variant,” “mutant,” and “mutein” refer to biologically active derivatives of the reference molecule that retain desired activity or are engineered for specific activity, such as the alpha-biased IL-2 variants described herein with increased affinity for CD25 and/or decreased affinity for CD122 and/or CD132. In general, the terms “variant” and “mutant” refer to compounds having a native polypeptide sequence and structure with one or more amino acid additions, substitutions (generally conservative in nature) and/or deletions, relative to the native molecule, so long as the modifications do not destroy biological activity, and which are “substantially homologous” to the reference molecule as defined below. In general, the amino acid sequences of such variants will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the variants will include the same number of amino acids but will include substitutions, as explained herein. The term “mutant” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like.

### DETAILED DESCRIPTION

The present invention provides, among other things, a method of prophylaxis for kidney rejection comprising administering to the recipient an alpha-biased IL-2 variant in conjunction with the kidney transplant. As described herein, the present invention is, in part, based on an unexpected discovery that a transplanted kidney remains stable for over 235 days when the recipient was administered with an alpha-biased IL-2 variant in conjunction with the kidney transplant. This is significant because rejection of the transplanted kidney by the immune system is a major challenge along with increased risks of infections and malignancies caused by currently approved therapies such as immunosuppressants. As demonstrated herein, the administration of an IL-2 variant of the present invention prolonged survival of the recipient and stabilized the transplanted kidney such that the recipient, among other things, maintained the creatinine level and blood urea nitrogen level within the normal threshold.

### Kidney Transplant

Kidney transplantation is a surgical procedure that involves placing a donor kidney from a living or deceased donor into a person whose own kidneys have lost the ability to properly filter waste and fluids from the body. This condition, known as kidney failure or end-stage renal disease (ESRD), can lead to the buildup of harmful levels of waste products in the body, as well as high blood pressure. Kidney transplantation is typically performed to treat kidney failure or ESRD, when the kidneys can no longer effectively filter waste. This procedure offers the best chance to restore normal kidney function and improve the patient's quality of life.

However, kidney transplantation faces long-term challenges, including graft failure and side effects from standard of care such as immunosuppressive treatments. One of the major risks is transplant rejection. Kidney transplant is currently followed by an immunosuppressive regimen, or standard of care, to reduce and treat transplant rejection. However, long term use of systemic immunosuppressants, while decreasing rejection, also weakens the immune system overall leaving the patient more susceptible to infection. Long-term, non-specific, immunosuppression can also lead to cardiovascular disease, malignancies, nephrotoxicity, and metabolic complications. Even with standard of care, such as, for example, immunosuppressive therapy, approximately 10-20% of patients will still experience at least one episode of rejection.

### Standard of Care for Kidney Transplant

Immunosuppressive agents are often prescribed as the standard of care for kidney transplantation. In some cases, standard of care involves two phases: a first induction phase to reduce the risk of acute rejection and a second maintenance phase to promote long-term stability of the kidney allograft. However, long-term use of immunosuppressive agents can lead to unwanted side effects and health complications which requires an approach that balances the benefits and risks of the immunosuppressive agents. As such, in some embodiments, the method provided herein allows for tapering, or slowly decreasing dosage over time, and/or withdrawal of the immunosuppressive agents.

In some embodiments, the standard of care comprises induction phase. In some embodiments, the standard of care comprises maintenance phase. In some embodiments, the standard of care comprises induction and maintenance phases.

In some embodiments, the standard of care comprises administration of one or more immunosuppressive agents. In some embodiments, the standard of care comprises administration of antithymocyte globulin (rATG). In some embodiments, the standard of care comprises administration of calcineurin inhibitors (CNI), mammalian target of rapamycin (mTOR) inhibitors, antiproliferative agents, and/or corticosteroids.

In some embodiments, the standard of care comprises administration of a calcineurin inhibitors (CNI). In some embodiments, the CNI is tacrolimus. In some embodiments, the CNI is cyclosporine A.

In some embodiments, the standard of care comprises administration of a mTOR inhibitor. In some embodiments, the mTOR inhibitor is rapamycin. In some embodiments, the mTOR inhibitor is everolimus. In some embodiments, the mTOR inhibitor is Sirolimus (Rapamune).

In some embodiments, the standard of care comprises administration of an antiproliferative agent. In some embodiments, the antiproliferative agent is azathioprine. In some embodiments, the antiproliferative agent is mycophenolate mofetil (MMF).

In some embodiments, the standard of care comprises administration of a corticosteroid. In some embodiments, the corticosteroid is prednisone. In some embodiments, the corticosteroid is methylprednisolone. In some embodiments, the corticosteroid is dexamethasone. In some embodiments, the corticosteroid is hydrocortisone.

### Measures of Kidney Transplant Outcomes

The outcome of kidney transplant is tracked by a few key measures. Renal function is determined by serum creatinine levels, blood urea nitrogen, and glomerular filtration rate (GFR). Abnormal kidney function is often associated with transplant rejection. Other than kidney function, testing for donor-specific antibodies or donor-derived free DNA, for example, can be used to suggest possible rejection.

### Creatinine Level

Creatinine is a waste product that is filtered out of the blood by the kidneys. Measuring the level of creatinine in the blood (serum creatinine) is a common way to assess kidney function. For example, serial creatinine measurements can track the progression or improvement of kidney disease over time. The standard normal range for creatinine in the blood is 0.7-1.3 mg/dL for men and 0.6-1.1 mg/dL for women, but this can also vary based on factors like age, and muscle mass. High levels in the blood might indicate that the kidneys are not working correctly, while low levels can occur with low muscle mass.

In some embodiments, a kidney transplant recipient administered with an alpha-biased IL-2 variant has a creatinine level below 4 mg/dl for at least two weeks, three weeks, one month, two months, three months, four months, six months, or a year. In some embodiments, a recipient has a creatinine level below 4 mg/dl for at least two weeks. In some embodiments, a recipient has a creatinine level below 4 mg/dl for at least 4 weeks. In some embodiments, a recipient has a creatinine level below 4 mg/dl for at least 5 weeks. In some embodiments, a recipient has a creatinine level below 4 mg/dl for at least 6 weeks. In some embodiments, a recipient has a creatinine level below 4 mg/dl for at least 7 weeks. In some embodiments, a recipient has a creatinine level below 4 mg/dl for at least 8 weeks. In some embodiments, a recipient has a creatinine level below 4 mg/dl for at least 10 weeks. In some embodiments, a recipient has a creatinine level below 4 mg/dl for at least 12 weeks. In some embodiments, a recipient has a creatinine level below 4 mg/dl for at least 14 weeks. In some embodiments, a recipient has a creatinine level below 4 mg/dl for at least 16 weeks. In some embodiments, a recipient has a creatinine level below 4 mg/dl for at least 18 weeks. In some embodiments, a recipient has a creatinine level below 4 mg/dl for at least 20 weeks. In some embodiments, a recipient has a creatinine level below 4 mg/dl for at least 25 weeks. In some embodiments, a recipient has a creatinine level below 4 mg/dl for at least 30 weeks. In some embodiments, a recipient has a creatinine level below 4 mg/dl for at least 35 weeks. In some embodiments, a recipient has a creatinine level below 4 mg/dl for at least 40 weeks. In some embodiments, a recipient has a creatinine level below 4 mg/dl for at least 50 weeks. In some embodiments, a recipient has a creatinine level below 4 mg/dl for at least one year. In some embodiments, a recipient has a creatinine level below 4 mg/dl for at least two years. In some embodiments, a recipient has a creatinine level below 4 mg/dl for at least five years.

In some embodiments, a kidney transplant recipient administered with an alpha-biased IL-2 variant has a creatinine level below 3 mg/dl for at least two weeks, three weeks, one month, two months, three months, four months, six months, or a year. In some embodiments, a recipient has a creatinine level below 3 mg/dl for at least two weeks. In some embodiments, a recipient has a creatinine level below 3 mg/dl for at least 4 weeks. In some embodiments, a recipient has a creatinine level below 3 mg/dl for at least 5 weeks. In some embodiments, a recipient has a creatinine level below 3 mg/dl for at least 6 weeks. In some embodiments, a recipient has a creatinine level below 3 mg/dl for at least 7 weeks. In some embodiments, a recipient has a creatinine level below 3 mg/dl for at least 8 weeks. In some embodiments, a recipient has a creatinine level below 3 mg/dl for at least 10 weeks. In some embodiments, a recipient has a creatinine level below 3 mg/dl for at least 12 weeks. In some embodiments, a recipient has a creatinine level below 3 mg/dl for at least 14 weeks. In some embodiments, a recipient has a creatinine level below 3 mg/dl for at least 16 weeks. In some embodiments, a recipient has a creatinine level below 3 mg/dl for at least 18 weeks. In some embodiments, a recipient has a creatinine level below 3 mg/dl for at least 20 weeks. In some embodiments, a recipient has a creatinine level below 3 mg/dl for at least 25 weeks. In some embodiments, a recipient has a creatinine level below 3 mg/dl for at least 30 weeks. In some embodiments, a recipient has a creatinine level below 3 mg/dl for at least 35 weeks. In some embodiments, a recipient has a creatinine level below 3 mg/dl for at least 40 weeks. In some embodiments, a recipient has a creatinine level below 3 mg/dl for at least 50 weeks. In some embodiments, a recipient has a creatinine level below 3 mg/dl for at least one year. In some embodiments, a recipient has a creatinine level below 3 mg/dl for at least two years. In some embodiments, a recipient has a creatinine level below 3 mg/dl for at least five years.

In some embodiments, a kidney transplant recipient administered with an alpha-biased IL-2 variant has a creatinine level below 2.5 mg/dl for at least two weeks, three weeks, one month, two months, three months, four months, six months, or a year. In some embodiments, a recipient has a creatinine level below 2.5 mg/dl for at least two weeks. In some embodiments, a recipient has a creatinine level below 2.5 mg/dl for at least 4 weeks. In some embodiments, a recipient has a creatinine level below 2.5 mg/dl for at least 5 weeks. In some embodiments, a recipient has a creatinine level below 2.5 mg/dl for at least 6 weeks. In some embodiments, a recipient has a creatinine level below 2.5 mg/dl for at least 7 weeks. In some embodiments, a recipient has a creatinine level below 2.5 mg/dl for at least 8 weeks. In some embodiments, a recipient has a creatinine level below 2.5 mg/dl for at least 10 weeks. In some embodiments, a recipient has a creatinine level below 2.5 mg/dl for at least 12 weeks. In some embodiments, a recipient has a creatinine level below 2.5 mg/dl for at least 14 weeks. In some embodiments, a recipient has a creatinine level below 2.5 mg/dl for at least 16 weeks. In some embodiments, a recipient has a creatinine level below 2.5 mg/dl for at least 18 weeks. In some embodiments, a recipient has a creatinine level below 2.5 mg/dl for at least 20 weeks. In some embodiments, a recipient has a creatinine level below 2.5 mg/dl for at least 25 weeks. In some embodiments, a recipient has a creatinine level below 2.5 mg/dl for at least 30 weeks. In some embodiments, a recipient has a creatinine level below 2.5 mg/dl for at least 35 weeks. In some embodiments, a recipient has a creatinine level below 2.5 mg/dl for at least 40 weeks. In some embodiments, a recipient has a creatinine level below 2.5 mg/dl for at least 50 weeks. In some embodiments, a recipient has a creatinine level below 2.5 mg/dl for at least one year. In some embodiments, a recipient has a creatinine level below 2.5 mg/dl for at least two years. In some embodiments, a recipient has a creatinine level below 2.5 mg/dl for at least five years.

In some embodiments, a kidney transplant recipient administered with an alpha-biased IL-2 variant has a creatinine level below 2 mg/dl for at least two weeks, three weeks, one month, two months, three months, four months, six months, or a year. In some embodiments, a recipient has a creatinine level below 2 mg/dl for at least two weeks. In some embodiments, a recipient has a creatinine level below 2 mg/dl for at least 4 weeks. In some embodiments, a recipient has a creatinine level below 2 mg/dl for at least 5 weeks. In some embodiments, a recipient has a creatinine level below 2 mg/dl for at least 6 weeks. In some embodiments, a recipient has a creatinine level below 2 mg/dl for at least 7 weeks. In some embodiments, a recipient has a creatinine level below 2 mg/dl for at least 8 weeks. In some embodiments, a recipient has a creatinine level below 2 mg/dl for at least 10 weeks. In some embodiments, a recipient has a creatinine level below 2 mg/dl for at least 12 weeks. In some embodiments, a recipient has a creatinine level below 2 mg/dl for at least 14 weeks. In some embodiments, a recipient has a creatinine level below 2 mg/dl for at least 16 weeks. In some embodiments, a recipient has a creatinine level below 2 mg/dl for at least 18 weeks. In some embodiments, a recipient has a creatinine level below 2 mg/dl for at least 20 weeks. In some embodiments, a recipient has a creatinine level below 2 mg/dl for at least 25 weeks. In some embodiments, a recipient has a creatinine level below 2 mg/dl for at least 30 weeks. In some embodiments, a recipient has a creatinine level below 2 mg/dl for at least 35 weeks. In some embodiments, a recipient has a creatinine level below 2 mg/dl for at least 40 weeks. In some embodiments, a recipient has a creatinine level below 2 mg/dl for at least 50 weeks. In some embodiments, a recipient has a creatinine level below 2 mg/dl for at least one year. In some embodiments, a recipient has a creatinine level below 2 mg/dl for at least two years. In some embodiments, a recipient has a creatinine level below 2 mg/dl for at least five years.

In some embodiments, a kidney transplant recipient administered with an alpha-biased IL-2 variant has a creatinine level below 1.8 mg/dl for at least two weeks, three weeks, one month, two months, three months, four months, six months, or a year. In some embodiments, a recipient has a creatinine level below 1.8 mg/dl for at least two weeks. In some embodiments, a recipient has a creatinine level below 1.8 mg/dl for at least 4 weeks. In some embodiments, a recipient has a creatinine level below 1.8 mg/dl for at least 5 weeks. In some embodiments, a recipient has a creatinine level below 1.8 mg/dl for at least 6 weeks. In some embodiments, a recipient has a creatinine level below 1.8 mg/dl for at least 7 weeks. In some embodiments, a recipient has a creatinine level below 1.8 mg/dl for at least 8 weeks. In some embodiments, a recipient has a creatinine level below 1.8 mg/dl for at least 10 weeks. In some embodiments, a recipient has a creatinine level below 1.8 mg/dl for at least 12 weeks. In some embodiments, a recipient has a creatinine level below 1.8 mg/dl for at least 14 weeks. In some embodiments, a recipient has a creatinine level below 1.8 mg/dl for at least 16 weeks. In some embodiments, a recipient has a creatinine level below 1.8 mg/dl for at least 18 weeks. In some embodiments, a recipient has a creatinine level below 1.8 mg/dl for at least 20 weeks. In some embodiments, a recipient has a creatinine level below 1.8 mg/dl for at least 25 weeks. In some embodiments, a recipient has a creatinine level below 1.8 mg/dl for at least 30 weeks. In some embodiments, a recipient has a creatinine level below 1.8 mg/dl for at least 35 weeks. In some embodiments, a recipient has a creatinine level below 1.8 mg/dl for at least 40 weeks. In some embodiments, a recipient has a creatinine level below 1.8 mg/dl for at least 50 weeks. In some embodiments, a recipient has a creatinine level below 1.8 mg/dl for at least one year. In some embodiments, a recipient has a creatinine level below 1.8 mg/dl for at least two years. In some embodiments, a recipient has a creatinine level below 1.8 mg/dl for at least five years.

In some embodiments, a kidney transplant recipient administered with an alpha-biased IL-2 variant has a creatinine level below 1.5 mg/dl for at least two weeks, three weeks, one month, two months, three months, four months, six months, or a year. In some embodiments, a recipient has a creatinine level below 1.5 mg/dl for at least two weeks. In some embodiments, a recipient has a creatinine level below 1.5 mg/dl for at least 4 weeks. In some embodiments, a recipient has a creatinine level below 1.5 mg/dl for at least 5 weeks. In some embodiments, a recipient has a creatinine level below 1.5 mg/dl for at least 6 weeks. In some embodiments, a recipient has a creatinine level below 1.5 mg/dl for at least 7 weeks. In some embodiments, a recipient has a creatinine level below 1.5 mg/dl for at least 8 weeks. In some embodiments, a recipient has a creatinine level below 1.5 mg/dl for at least 10 weeks. In some embodiments, a recipient has a creatinine level below 1.5 mg/dl for at least 12 weeks. In some embodiments, a recipient has a creatinine level below 1.5 mg/dl for at least 14 weeks. In some embodiments, a recipient has a creatinine level below 1.5 mg/dl for at least 16 weeks. In some embodiments, a recipient has a creatinine level below 1.5 mg/dl for at least 18 weeks. In some embodiments, a recipient has a creatinine level below 1.5 mg/dl for at least 20 weeks. In some embodiments, a recipient has a creatinine level below 1.5 mg/dl for at least 25 weeks. In some embodiments, a recipient has a creatinine level below 1.5 mg/dl for at least 30 weeks. In some embodiments, a recipient has a creatinine level below 1.5 mg/dl for at least 35 weeks. In some embodiments, a recipient has a creatinine level below 1.5 mg/dl for at least 40 weeks. In some embodiments, a recipient has a creatinine level below 1.5 mg/dl for at least 50 weeks. In some embodiments, a recipient has a creatinine level below 1.5 mg/dl for at least one year. In some embodiments, a recipient has a creatinine level below 1.5 mg/dl for at least two years. In some embodiments, a recipient has a creatinine level below 1.5 mg/dl for at least five years.

In some embodiments, a kidney transplant recipient administered with an alpha-biased IL-2 variant has a creatinine level between 0.3-2 mg/dl for at least two weeks, three weeks, one month, two months, three months, four months, six months, or a year. In some embodiments, a recipient has a creatinine level between 0.3-2 mg/dl for at least two weeks. In some embodiments, a recipient has a creatinine level between 0.3-2 mg/dl for at least 4 weeks. In some embodiments, a recipient has a creatinine level between 0.3-2 mg/dl for at least 5 weeks. In some embodiments, a recipient has a creatinine level between 0.3-2 mg/dl for at least 6 weeks. In some embodiments, a recipient has a creatinine level between 0.3-2 mg/dl for at least 7 weeks. In some embodiments, a recipient has a creatinine level between 0.3-2 mg/dl for at least 8 weeks. In some embodiments, a recipient has a creatinine level between 0.3-2 mg/dl for at least 10 weeks. In some embodiments, a recipient has a creatinine level between 0.3-2 mg/dl for at least 12 weeks. In some embodiments, a recipient has a creatinine level between 0.3-2 mg/dl for at least 14 weeks. In some embodiments, a recipient has a creatinine level between 0.3-2 mg/dl for at least 16 weeks. In some embodiments, a recipient has a creatinine level between 0.3-2 mg/dl for at least 18 weeks. In some embodiments, a recipient has a creatinine level between 0.3-2 mg/dl for at least 20 weeks. In some embodiments, a recipient has a creatinine level between 0.3-2 mg/dl for at least 25 weeks. In some embodiments, a recipient has a creatinine level between 0.3-2 mg/dl for at least 30 weeks. In some embodiments, a recipient has a creatinine level between 0.3-2 mg/dl for at least 35 weeks. In some embodiments, a recipient has a creatinine level between 0.3-2 mg/dl for at least 40 weeks. In some embodiments, a recipient has a creatinine level between 0.3-2 mg/dl for at least 50 weeks. In some embodiments, a recipient has a creatinine level between 0.3-2 mg/dl for at least one year. In some embodiments, a recipient has a creatinine level between 0.3-2 mg/dl for at least two years. In some embodiments, a recipient has a creatinine level between 0.6-1.1 mg/dl for at least five years.

In some embodiments, a kidney transplant recipient administered with an alpha-biased IL-2 variant has a creatinine level between 0.5-1.6 for at least two weeks, three weeks, one month, two months, three months, four months, six months, or a year. In some embodiments, a recipient has a creatinine level between 0.5-1.6 mg/dl for at least two weeks. In some embodiments, a recipient has a creatinine level between 0.5-1.6 mg/dl for at least 4 weeks. In some embodiments, a recipient has a creatinine level between 0.5-1.6 mg/dl for at least 5 weeks. In some embodiments, a recipient has a creatinine level between 0.5-1.6 mg/dl for at least 6 weeks. In some embodiments, a recipient has a creatinine level between 0.5-1.6 mg/dl for at least 7 weeks. In some embodiments, a recipient has a creatinine level between 0.5-1.6 mg/dl for at least 8 weeks. In some embodiments, a recipient has a creatinine level between 0.5-1.6 mg/dl for at least 10 weeks. In some embodiments, a recipient has a creatinine level between 0.5-1.6 mg/dl for at least 12 weeks. In some embodiments, a recipient has a creatinine level between 0.5-1.6 mg/dl for at least 14 weeks. In some embodiments, a recipient has a creatinine level between 0.5-1.6 mg/dl for at least 16 weeks. In some embodiments, a recipient has a creatinine level between 0.5-1.6 mg/dl for at least 18 weeks. In some embodiments, a recipient has a creatinine level between 0.5-1.6 mg/dl for at least 20 weeks. In some embodiments, a recipient has a creatinine level between 0.5-1.6 mg/dl for at least 25 weeks. In some embodiments, a recipient has a creatinine level between 0.5-1.6 mg/dl for at least 30 weeks. In some embodiments, a recipient has a creatinine level between 0.5-1.6 mg/dl for at least 35 weeks. In some embodiments, a recipient has a creatinine level between 0.5-1.6 mg/dl for at least 40 weeks. In some embodiments, a recipient has a creatinine level between 0.5-1.6 mg/dl for at least 50 weeks. In some embodiments, a recipient has a creatinine level between 0.5-1.6 mg/dl for at least one year. In some embodiments, a recipient has a creatinine level between 0.5-1.6 mg/dl for at least two years. In some embodiments, a recipient has a creatinine level between 0.5-1.6 mg/dl for at least five years.

### Blood Urea Nitrogen (BUN) Level

BUN measures the amount of urea nitrogen in the blood. Urea nitrogen is another waste product normally filtered out by the kidneys. The normal range of blood urea nitrogen (BUN) is around 7-20 mg/dL for children, 6-21 mg/dL in adult women, and 8-24 mg/dL in adult men. Elevated BUN levels can indicate impaired kidney function, as the kidneys are not properly filtering out urea from the blood. Severely elevated BUN levels, often over 50-100 mg/dL, indicate advanced kidney dysfunction and damage that requires immediate medical attention.

In some embodiments, a kidney transplant recipient administered with an alpha-biased IL-2 variant has a BUN level below 45 mg/dl for at least two weeks, three weeks, one month, two months, three months, four months, six months, or a year. In some embodiments, a recipient has a BUN level below 45 mg/dl for at least two weeks. In some embodiments, a recipient has a BUN level below 45 mg/dl for at least 4 weeks. In some embodiments, a recipient has a BUN level below 45 mg/dl for at least 5 weeks. In some embodiments, a recipient has a BUN level below 45 mg/dl for at least 6 weeks. In some embodiments, a recipient has a BUN level below 45 mg/dl for at least 7 weeks. In some embodiments, a recipient has a BUN level below 45 mg/dl for at least 8 weeks. In some embodiments, a recipient has a BUN level below 45 mg/dl for at least 10 weeks. In some embodiments, a recipient has a BUN level below 45 mg/dl for at least 12 weeks. In some embodiments, a recipient has a BUN level below 45 mg/dl for at least 14 weeks. In some embodiments, a recipient has a BUN level below 45 mg/dl for at least 16 weeks. In some embodiments, a recipient has a BUN level below 45 mg/dl for at least 18 weeks. In some embodiments, a recipient has a BUN level below 45 mg/dl for at least 20 weeks. In some embodiments, a recipient has a BUN level below 45 mg/dl for at least 25 weeks. In some embodiments, a recipient has a BUN level below 45 mg/dl for at least 30 weeks. In some embodiments, a recipient has a BUN level below 45 mg/dl for at least 35 weeks. In some embodiments, a recipient has a BUN level below 45 mg/dl for at least 40 weeks. In some embodiments, a recipient has a BUN level below 45 mg/dl for at least 50 weeks. In some embodiments, a recipient has a BUN level below 45 mg/dl for at least one year. In some embodiments, a recipient has a BUN level below 45 mg/dl for at least two years. In some embodiments, a recipient has a BUN level below 45 mg/dl for at least five years.

In some embodiments, a kidney transplant recipient administered with an alpha-biased IL-2 variant has a BUN level below 40 mg/dl for at least two weeks, three weeks, one month, two months, three months, four months, six months, or a year. In some embodiments, a recipient has a BUN level below 40 mg/dl for at least two weeks. In some embodiments, a recipient has a BUN level below 40 mg/dl for at least 4 weeks. In some embodiments, a recipient has a BUN level below 40 mg/dl for at least 5 weeks. In some embodiments, a recipient has a BUN level below 40 mg/dl for at least 6 weeks. In some embodiments, a recipient has a BUN level below 40 mg/dl for at least 7 weeks. In some embodiments, a recipient has a BUN level below 40 mg/dl for at least 8 weeks. In some embodiments, a recipient has a BUN level below 40 mg/dl for at least 10 weeks. In some embodiments, a recipient has a BUN level below 40 mg/dl for at least 12 weeks. In some embodiments, a recipient has a BUN level below 40 mg/dl for at least 14 weeks. In some embodiments, a recipient has a BUN level below 40 mg/dl for at least 16 weeks. In some embodiments, a recipient has a BUN level below 40 mg/dl for at least 18 weeks. In some embodiments, a recipient has a BUN level below 40 mg/dl for at least 20 weeks. In some embodiments, a recipient has a BUN level below 40 mg/dl for at least 25 weeks. In some embodiments, a recipient has a BUN level below 40 mg/dl for at least 30 weeks. In some embodiments, a recipient has a BUN level below 40 mg/dl for at least 35 weeks. In some embodiments, a recipient has a BUN level below 40 mg/dl for at least 40 weeks. In some embodiments, a recipient has a BUN level below 40 mg/dl for at least 50 weeks. In some embodiments, a recipient has a BUN level below 40 mg/dl for at least one year. In some embodiments, a recipient has a BUN level below 40 mg/dl for at least two years. In some embodiments, a recipient has a BUN level below 40 mg/dl for at least five years.

In some embodiments, a kidney transplant recipient administered with an alpha-biased IL-2 variant has a BUN level below 30 mg/dl for at least two weeks, three weeks, one month, two months, three months, four months, six months, or a year. In some embodiments, a recipient has a BUN level below 30 mg/dl for at least two weeks. In some embodiments, a recipient has a BUN level below 30 mg/dl for at least 4 weeks. In some embodiments, a recipient has a BUN level below 30 mg/dl for at least 5 weeks. In some embodiments, a recipient has a BUN level below 30 mg/dl for at least 6 weeks. In some embodiments, a recipient has a BUN level below 30 mg/dl for at least 7 weeks. In some embodiments, a recipient has a BUN level below 30 mg/dl for at least 8 weeks. In some embodiments, a recipient has a BUN level below 30 mg/dl for at least 10 weeks. In some embodiments, a recipient has a BUN level below 30 mg/dl for at least 12 weeks. In some embodiments, a recipient has a BUN level below 30 mg/dl for at least 14 weeks. In some embodiments, a recipient has a BUN level below 30 mg/dl for at least 16 weeks. In some embodiments, a recipient has a BUN level below 30 mg/dl for at least 18 weeks. In some embodiments, a recipient has a BUN level below 30 mg/dl for at least 20 weeks. In some embodiments, a recipient has a BUN level below 30 mg/dl for at least 25 weeks. In some embodiments, a recipient has a BUN level below 30 mg/dl for at least 30 weeks. In some embodiments, a recipient has a BUN level below 30 mg/dl for at least 35 weeks. In some embodiments, a recipient has a BUN level below 30 mg/dl for at least 40 weeks. In some embodiments, a recipient has a BUN level below 30 mg/dl for at least 50 weeks. In some embodiments, a recipient has a BUN level below 30 mg/dl for at least one year. In some embodiments, a recipient has a BUN level below 30 mg/dl for at least two years. In some embodiments, a recipient has a BUN level below 30 mg/dl for at least five years.

In some embodiments, a kidney transplant recipient administered with an alpha-biased IL-2 variant has a BUN level below 25 mg/dl for at least two weeks, three weeks, one month, two months, three months, four months, six months, or a year. In some embodiments, a recipient has a BUN level below 25 mg/dl for at least two weeks. In some embodiments, a recipient has a BUN level below 25 mg/dl for at least 4 weeks. In some embodiments, a recipient has a BUN level below 25 mg/dl for at least 5 weeks. In some embodiments, a recipient has a BUN level below 25 mg/dl for at least 6 weeks. In some embodiments, a recipient has a BUN level below 25 mg/dl for at least 7 weeks. In some embodiments, a recipient has a BUN level below 25 mg/dl for at least 8 weeks. In some embodiments, a recipient has a BUN level below 25 mg/dl for at least 10 weeks. In some embodiments, a recipient has a BUN level below 25 mg/dl for at least 12 weeks. In some embodiments, a recipient has a BUN level below 25 mg/dl for at least 14 weeks. In some embodiments, a recipient has a BUN level below 25 mg/dl for at least 16 weeks. In some embodiments, a recipient has a BUN level below 25 mg/dl for at least 18 weeks. In some embodiments, a recipient has a BUN level below 25 mg/dl for at least 20 weeks. In some embodiments, a recipient has a BUN level below 25 mg/dl for at least 25 weeks. In some embodiments, a recipient has a BUN level below 25 mg/dl for at least 30 weeks. In some embodiments, a recipient has a BUN level below 25 mg/dl for at least 35 weeks. In some embodiments, a recipient has a BUN level below 25 mg/dl for at least 40 weeks. In some embodiments, a recipient has a BUN level below 25 mg/dl for at least 50 weeks. In some embodiments, a recipient has a BUN level below 25 mg/dl for at least one year. In some embodiments, a recipient has a BUN level below 25 mg/dl for at least two years. In some embodiments, a recipient has a BUN level below 25 mg/dl for at least five years.

In some embodiments, a kidney transplant recipient administered with an alpha-biased IL-2 variant has a BUN level between 5-25 mg/dl for at least two weeks, three weeks, one month, two months, three months, four months, six months, or a year. In some embodiments, a recipient has a BUN level between 5-25 mg/dl for at least two weeks. In some embodiments, a recipient has a BUN level between 5-25 mg/dl for at least 4 weeks. In some embodiments, a recipient has a BUN level between 5-25 mg/dl for at least 5 weeks. In some embodiments, a recipient has a BUN level between 5-25 mg/dl for at least 6 weeks. In some embodiments, a recipient has a BUN level between 5-25 mg/dl for at least 7 weeks. In some embodiments, a recipient has a BUN level between 5-25 mg/dl for at least 8 weeks. In some embodiments, a recipient has a BUN level between 5-25 mg/dl for at least 10 weeks. In some embodiments, a recipient has a BUN level between 5-25 mg/dl for at least 12 weeks. In some embodiments, a recipient has a BUN level between 5-25 mg/dl for at least 14 weeks. In some embodiments, a recipient has a BUN level between 5-25 mg/dl for at least 16 weeks. In some embodiments, a recipient has a BUN level between 5-25 mg/dl for at least 18 weeks. In some embodiments, a recipient has a BUN level between 5-25 mg/dl for at least 20 weeks. In some embodiments, a recipient has a BUN level between 5-25 mg/dl for at least 25 weeks. In some embodiments, a recipient has a BUN level between 5-25 mg/dl for at least 30 weeks. In some embodiments, a recipient has a BUN level between 5-25 mg/dl for at least 35 weeks. In some embodiments, a recipient has a BUN level between 5-25 mg/dl for at least 40 weeks. In some embodiments, a recipient has a BUN level between 5-25 mg/dl for at least 50 weeks. In some embodiments, a recipient has a BUN level between 5-25 mg/dl for at least one year. In some embodiments, a recipient has a BUN level between 5-25 mg/dl for at least two years. In some embodiments, a recipient has a BUN level between 5-25 mg/dl for at least five years.

### GFR (Glomerular Filtration Rate)

The GFR (glomerular filtration rate) is the calculated percent of kidney function in a patient as compared to normal. Higher levels of serum creatinine or impaired GFR, lower levels of calculated kidney function, can be used as early identifies for graft damage or dysfunction. A normal GFR is typically 90-120 mL/min/1.73 m2 in young healthy adults. GFR naturally declines with age. A GFR below 60 mL/min/1.73 m2 for 3 or more months indicates chronic kidney disease. A GFR below 15 mL/min/1.73 m2 indicates kidney failure and the need for dialysis or transplantation.

### Alpha-Biased IL-2 Variants

IL-2 is an important cytokine for the activation, growth, and differentiation of T cells, and it plays a major role in T cell stimulation and B cell proliferation and maturation. The IL-2 receptor, with its different affinity forms, mediates diverse biological functions of IL-2 in the immune system, including T cell activation, proliferation, and regulation of immune responses. The IL-2 receptor consists of one or more subunits selected from: IL-2Rβ (CD122), IL-2Rγ (CD132), and IL-2Rα (CD25). The dimeric IL-2 receptor, comprising CD122 and CD132, is common to cytotoxic T cells and natural killer (NK) cells which participate in the adaptive and innate immune response, respectively. On the other hand, the trimeric IL-2 receptor, further comprising CD25, is associated with regulatory T cells (Tregs) which promote immune tolerance.

Alpha-biased IL-2 variants are IL-2 variants with mutations that increase binding to IL-2Rα (CD25), decrease binding to IL-2Rβ (CD122) and/or IL-2Rγ (CD132). Without wishing to be bound to any particular theory, it is thought that alpha-biased IL-2 variants preferentially bind to the trimeric IL-2 receptor, which contains CD25. The preferential binding to the trimeric receptor results in preferential binding to and activation of Tregs, which can induce immune tolerance and reduce transplant rejection.

In some embodiments, the alpha-biased IL-2 variant comprises increased affinity to CD25 as compared to wild-type IL-2. In some embodiments, the alpha-biased IL-2 variant comprises increased affinity to CD25 as compared to its affinity to CD122 and/or CD132. In some embodiments, the alpha-biased IL-2 variant comprises decreased affinity to CD122 and/or CD132 as compared to wild-type IL-2. In some embodiments, the alpha-biased IL-2 variant comprises decreased affinity to CD122 and CD132 as compared to wild-type IL-2. In some embodiments, the alpha-biased IL-2 variant comprises decreased affinity to CD122 as compared to wild-type IL-2. In some embodiments, the alpha-biased IL-2 variant comprises decreased affinity to CD132 as compared to wild-type IL-2. In some embodiments, the alpha-biased IL-2 variant comprises decreased affinity to the CD122/CD132 heterodimer as compared to wild-type IL-2. In some embodiments, the alpha-biased IL-2 variant preferentially binds to the trimeric IL-2 receptor over the dimeric IL-2 receptor.

In some embodiments, the alpha-biased IL-2 variant is further engineered to increase protein stability and expression. In some embodiments, the alpha-biased IL-2 variant is further engineered to increase protein stability. In some embodiments, the alpha-biased IL-2 variant is further engineered to increase protein expression.

In some embodiments, two alpha-biased IL-2 variants are fused together. In some embodiments, three alpha-biased IL-2 variants are fused together. In some embodiments, four alpha-biased IL-2 variants are fused together. In some embodiments, five alpha-biased IL-2 variants are fused together. In some embodiments, greater than two alpha-biased IL-2 variants are fused together.

### Exemplary Alpha-Biased IL-2 Variants

As described herein, all alpha-biased IL-2 variants are also considered IL-2 variants. In some embodiments, the alpha-biased IL-2 comprises one or more amino acid substitutions selected from Table 1. In some embodiments, the alpha-biased IL-2 variant further comprises the amino acid substitutions of V69A and Q74P. In some embodiments, the alpha-biased IL-2 variant further comprises the amino acid substitution of C125S.

TABLE 1
Exemplary Alpha-Biased IL-2 Mutations

| Position | Substitution | Position | Substitution |
|---|---|---|---|
| Q11 | E, R | Q74 | P |
| L12 | G, K, Q, S, D | H79 | R |
| Q13 | G | R81 | A, G, S, T |
| E15 | A, G, S | D84 | V, N, A, E, G, I, |
| | | | M, Q, R, S, T |
| H16 | D, L, N, A, G, K, | S87 | R, T |
| | M, R, S, T, V, Y | | |
| L19 | H, Y, N, R, Q, D, | N88 | S, L, D, A, E, F, |
| | P, S, A, E, G, T, V | | G, M, R, V, W, |
| | | | I, Q, M, T |
| D20 | T, E, N, Q, S, Y, | N90 | H |
| | I, A, F, G, W | | |
| L21 | S, N, R | V91 | K, D, E, G, S, L |
| Q22 | N, H, K, Y, I | I92 | S, K, R, L |
| M23 | R | E95 | G, Q |
| I24 | L | T101 | R, A |
| I28 | T, F | F103 | S |
| P34 | T, R | D109 | N |
| R38 | I | I114 | V |
| T41 | K, Q | L118 | I |
| M46 | L | N119 | D |
| K48 | E | R120 | G, D |
| K49 | R | T123 | S, A |
| E61 | D | C125 | E, K, H, W, I, |
| | | | A, S, V |
| K64 | R | Q126 | R, T, K, E, L, |
| | | | N, D, M, H, Y |
| E68 | D, S | I128 | T |
| V69 | L, A | S130 | T |
| N71 | T | T133 | S, N |

In some embodiments, the alpha-biased IL-2 variant comprises a deletion of the alanine at position 1. In some embodiments, the alpha-biased IL-2 variant comprises an amino acid substitution at the T3 position. In some embodiments, the IL-2 variant alpha-biased comprises an amino acid substitution of T3A, T3R, T3N, T3D, T3C, T3E, T3Q, T3G, T3H, T3I, T3L, T3K, T3M, T3F, T3P, T3W, T3Y, or T3V. In some embodiments, the IL-2 alpha-biased variant comprises an amino acid substitution of T3A. In some embodiments, the alpha-biased IL-2 variant comprises an amino acid substitution of T3R. In some embodiments, the alpha-biased IL-2 variant comprises an amino acid substitution of T3N. In some embodiments, the alpha-biased IL-2 variant comprises an amino acid substitution of T3D. In some embodiments, the alpha-biased IL-2 variant comprises an amino acid substitution of T3C. In some embodiments, the alpha-biased IL-2 variant comprises an amino acid substitution of T3E. In some embodiments, the alpha-biased IL-2 variant comprises an amino acid substitution of T3Q. In some embodiments, the alpha-biased IL-2 variant comprises an amino acid substitution of T3G. In some embodiments, the alpha-biased IL-2 variant comprises an amino acid substitution of T3H. In some embodiments, the alpha-biased IL-2 variant comprises an amino acid substitution of T3I. In some embodiments, the alpha-biased IL-2 variant comprises an amino acid substitution of T3L. In some embodiments, the alpha-biased IL-2 variant comprises an amino acid substitution of T3K. In some embodiments, the alpha-biased IL-2 variant comprises an amino acid substitution of T3M. In some embodiments, the alpha-biased IL-2 variant comprises an amino acid substitution of T3F. In some embodiments, the alpha-biased IL-2 variant comprises an amino acid substitution of T3P. In some embodiments, the alpha-biased IL-2 variant comprises an amino acid substitution of T3W. In some embodiments, the alpha-biased IL-2 variant comprises an amino acid substitution of T3Y. In some embodiments, the alpha-biased IL-2 variant comprises an amino acid substitution of T3V.

In some embodiments, the alpha-biased IL-2 variant comprises an amino acid substitution at the Q11 position. In some embodiments, the alpha-biased IL-2 variant comprises an amino acid substitution of Q11E. In some embodiments, the alpha-biased IL-2 variant comprises an amino acid substitution of Q11R.

In some embodiments, the alpha-biased IL-2 variant comprises an amino acid substitution at the L12 position. In some embodiments, the alpha-biased IL-2 variant comprises an amino acid substitution of L12G, L12K, L12Q, L12S, or L12D. In some embodiments, the alpha-biased IL-2 variant comprises an amino acid substitution of L12G. In some embodiments, the alpha-biased IL-2 variant comprises an amino acid substitution of L12K. In some embodiments, the alpha-biased IL-2 variant comprises an amino acid substitution of L12Q. In some embodiments, the alpha-biased IL-2 variant comprises an amino acid substitution of L12S. In some embodiments, the alpha-biased IL-2 variant comprises an amino acid substitution of L12D.

In some embodiments, the alpha-biased IL-2 variant comprises an amino acid substitution at the Q13 position. In some embodiments, the alpha-biased IL-2 variant comprises an amino acid substitution of Q13G.

In some embodiments, the alpha-biased IL-2 variant comprises an amino acid substitution at the E15 position. In some embodiments, the alpha-biased IL-2 variant comprises an amino acid substitution of E15A, E15G, or E15S. In some embodiments, the alpha-biased IL-2 variant comprises an amino acid substitution of E15A. In some embodiments, the alpha-biased IL-2 variant comprises an amino acid substitution of E15G. In some embodiments, the alpha-biased IL-2 variant comprises an amino acid substitution of E15S.

In some embodiments, the alpha-biased IL-2 variant comprises an amino acid substitution at the H16 position. In some embodiments, the alpha-biased IL-2 variant comprises an amino acid substitution of H16L, H16N, H16D, H16A, H16G, H16K, H16M, H16R, H16S, H16T, H16V, or H16Y. In some embodiments, the alpha-biased IL-2 variant comprises an amino acid substitution of H16L. In some embodiments, the alpha-biased IL-2 variant comprises an amino acid substitution of H16N. In some embodiments, the alpha-biased IL-2 variant comprises an amino acid substitution of H16D. In some embodiments, the alpha-biased IL-2 variant comprises an amino acid substitution of H16A. In some embodiments, the alpha-biased IL-2 variant comprises an amino acid substitution of H16G. In some embodiments, the alpha-biased IL-2 variant comprises an amino acid substitution of H16K, In some embodiments, the alpha-biased IL-2 variant comprises an amino acid substitution of H16M. In some embodiments, the alpha-biased IL-2 variant comprises an amino acid substitution of H16R. In some embodiments, the alpha-biased IL-2 variant comprises an amino acid substitution of H16S. In some embodiments, the alpha-biased IL-2 variant comprises an amino acid substitution of H16T. In some embodiments, the alpha-biased IL-2 variant comprises an amino acid substitution of H16V. In some embodiments, the alpha-biased IL-2 variant comprises an amino acid substitution of H16Y.

In some embodiments, the alpha-biased IL-2 variant comprises an amino acid substitution at position L19. In some embodiments, the alpha-biased IL-2 variant comprises an amino acid substitution of L19H, L19Y, L19N, L19R, L19Q, L19D, L19P, L19S, L19A, L19E, L19G, L19T, or L19V. In some embodiments, the alpha-biased IL-2 variant comprises an amino acid substitution of L19H. In some embodiments, the alpha-biased IL-2 variant comprises an amino acid substitution of L19Y. In some embodiments, the alpha-biased IL-2 variant comprises an amino acid substitution of L19N. In some embodiments, the alpha-biased IL-2 variant comprises an amino acid substitution of L19R. In some embodiments, the alpha-biased IL-2 variant comprises an amino acid substitution of L19Q. In some embodiments, the alpha-biased IL-2 variant comprises an amino acid substitution of L19D. In some embodiments, the alpha-biased IL-2 variant comprises an amino acid substitution of L19P. In some embodiments, the alpha-biased IL-2 variant comprises an amino acid substitution of L19S. In some embodiments, the alpha-biased IL-2 variant comprises an amino acid substitution of L19A. In some embodiments, the alpha-biased IL-2 variant comprises an amino acid substitution of L19E. In some embodiments, the alpha-biased IL-2 variant comprises an amino acid substitution of L19G. In some embodiments, the alpha-biased IL-2 variant comprises an amino acid substitution of L19T. In some embodiments, the alpha-biased IL-2 variant comprises an amino acid substitution of L19V.

In some embodiments, the alpha-biased IL-2 variant comprises an amino acid substitution at position D20. In some embodiments, the alpha-biased IL-2 variant comprises an amino acid substitution of D20T, D20E, D20N, D20Q, D20S, D20Y, D20I, D20A, D20F, D20G, or D20W. In some embodiments, the alpha-biased IL-2 variant comprises an amino acid substitution of D20T. In some embodiments, the alpha-biased IL-2 variant comprises an amino acid substitution of D20E. In some embodiments, the alpha-biased IL-2 variant comprises an amino acid substitution of D20N. In some embodiments, the alpha-biased IL-2 variant comprises an amino acid substitution of D20Q. In some embodiments, the alpha-biased IL-2 variant comprises an amino acid substitution of D20S. In some embodiments, the alpha-biased IL-2 variant comprises an amino acid substitution of D20Y. In some embodiments, the alpha-biased IL-2 variant comprises an amino acid substitution of D20I. In some embodiments, the alpha-biased IL-2 variant comprises an amino acid substitution of D20A. In some embodiments, the alpha-biased IL-2 variant comprises an amino acid substitution of D20F. In some embodiments, the alpha-biased IL-2 variant comprises an amino acid substitution of D20G. In some embodiments, the alpha-biased IL-2 variant comprises an amino acid substitution of D20W.

In some embodiments, the alpha-biased IL-2 variant comprises an amino acid substitution at position L21. In some embodiments, the alpha-biased IL-2 variant comprises an amino acid substitution of L21S, L21N, or L21R. In some embodiments, the alpha-biased IL-2 variant comprises an amino acid substitution of L21S. In some embodiments, the alpha-biased IL-2 variant comprises an amino acid substitution of L21N. In some embodiments, the alpha-biased IL-2 variant comprises an amino acid substitution of L21R.

In some embodiments, the alpha-biased IL-2 variant comprises an amino acid substitution at position Q22. In some embodiments, the alpha-biased IL-2 variant comprises an amino acid substitution of Q22N, Q22H, Q22K, Q22Y, or Q22I. In some embodiments, the alpha-biased IL-2 variant comprises an amino acid substitution of Q22N. In some embodiments, the alpha-biased IL-2 variant comprises an amino acid substitution of Q22H. In some embodiments, the alpha-biased IL-2 variant comprises an amino acid substitution of Q22K. In some embodiments, the alpha-biased IL-2 variant comprises an amino acid substitution of Q22Y. In some embodiments, the alpha-biased IL-2 variant comprises an amino acid substitution of Q22I.

In some embodiments, the alpha-biased IL-2 variant comprises an amino acid substitution at position M23. In some embodiments, the alpha-biased IL-2 variant comprises an amino acid substitution of M23R.

In some embodiments, the alpha-biased IL-2 variant comprises an amino acid substitution at position I24. In some embodiments, the alpha-biased IL-2 variant comprises an amino acid substitution of I24L.

In some embodiments, the alpha-biased IL-2 variant comprises an amino acid substitution at position I28. In some embodiments, the alpha-biased IL-2 variant comprises an amino acid substitution of I28T. In some embodiments, the alpha-biased IL-2 variant comprises an amino acid substitution of I28F.

In some embodiments, the alpha-biased IL-2 variant comprises an amino acid substitution at position P34. In some embodiments, the alpha-biased IL-2 variant comprises an amino acid substitution of P34T. In some embodiments, the alpha-biased IL-2 variant comprises an amino acid substitution of P34R.

In some embodiments, the alpha-biased IL-2 variant comprises an amino acid substitution at position R38. In some embodiments, the alpha-biased IL-2 variant comprises an amino acid substitution of R38I.

In some embodiments, the alpha-biased IL-2 variant comprises an amino acid substitution at position T41. In some embodiments, the alpha-biased IL-2 variant comprises an amino acid substitution of T41K. In some embodiments, the alpha-biased IL-2 variant comprises an amino acid substitution of T41Q.

In some embodiments, the alpha-biased IL-2 variant comprises an amino acid substitution at position M46. In some embodiments, the alpha-biased IL-2 variant comprises an amino acid substitution of M46L.

In some embodiments, the alpha-biased IL-2 variant comprises an amino acid substitution at position K48. In some embodiments, the alpha-biased IL-2 variant comprises an amino acid substitution of K48E.

In some embodiments, the alpha-biased IL-2 variant comprises an amino acid substitution at position K49. In some embodiments, the alpha-biased IL-2 variant comprises an amino acid substitution of K49R.

In some embodiments, the alpha-biased IL-2 variant comprises an amino acid substitution at position E61. In some embodiments, the alpha-biased IL-2 variant comprises an amino acid substitution of E61D.

In some embodiments, the alpha-biased IL-2 variant comprises an amino acid substitution at position K64. In some embodiments, the alpha-biased IL-2 variant comprises an amino acid substitution of K64R.

In some embodiments, the alpha-biased IL-2 variant comprises an amino acid substitution at position E68. In some embodiments, the alpha-biased IL-2 variant comprises an amino acid substitution of E68D. In some embodiments, the alpha-biased IL-2 variant comprises an amino acid substitution of E68S.

In some embodiments, the alpha-biased IL-2 variant comprises an amino acid substitution at position V69. In some embodiments, the alpha-biased IL-2 variant comprises an amino acid substitution of V69A. In some embodiments, the alpha-biased IL-2 variant comprises an amino acid substitution of V69A.

In some embodiments, the alpha-biased IL-2 variant comprises an amino acid substitution at position N71. In some embodiments, the alpha-biased IL-2 variant comprises an amino acid substitution of N71T.

In some embodiments, the alpha-biased IL-2 variant comprises an amino acid substitution at position Q74. In some embodiments, the alpha-biased IL-2 variant comprises an amino acid substitution of Q74P.

In some embodiments, the alpha-biased IL-2 variant comprises an amino acid substitution at position H79. In some embodiments, the alpha-biased IL-2 variant comprises an amino acid substitution of H79R.

In some embodiments, the alpha-biased IL-2 variant comprises an amino acid substitution at position R81. In some embodiments, the alpha-biased IL-2 variant comprises an amino acid substitution of R81A. In some embodiments, the alpha-biased IL-2 variant comprises an amino acid substitution of R81G. In some embodiments, the alpha-biased IL-2 variant comprises an amino acid substitution of R81S. In some embodiments, the alpha-biased IL-2 variant comprises an amino acid substitution of R81T.

In some embodiments, the alpha-biased IL-2 variant comprises an amino acid substitution at position D84. In some embodiments, the alpha-biased IL-2 variant comprises an amino acid substitution of D84V. In some embodiments, the alpha-biased IL-2 variant comprises an amino acid substitution of D84N. In some embodiments, the alpha-biased IL-2 variant comprises an amino acid substitution of D84A. In some embodiments, the alpha-biased IL-2 variant comprises an amino acid substitution of D84E. In some embodiments, the alpha-biased IL-2 variant comprises an amino acid substitution of D84G. In some embodiments, the alpha-biased IL-2 variant comprises an amino acid substitution of D84I. In some embodiments, the alpha-biased IL-2 variant comprises an amino acid substitution of D84M. In some embodiments, the alpha-biased IL-2 variant comprises an amino acid substitution of D84Q. In some embodiments, the alpha-biased IL-2 variant comprises an amino acid substitution of D84R. In some embodiments, the alpha-biased IL-2 variant comprises an amino acid substitution of D84S. In some embodiments, the alpha-biased IL-2 variant comprises an amino acid substitution of D84T.

In some embodiments, the alpha-biased IL-2 variant comprises an amino acid substitution at position S87. In some embodiments, the alpha-biased IL-2 variant comprises an amino acid substitution of S87R. In some embodiments, the alpha-biased IL-2 variant comprises an amino acid substitution of S87T.

In some embodiments, the alpha-biased IL-2 variant comprises an amino acid substitution at position N88. In some embodiments, the alpha-biased IL-2 variant comprises an amino acid substitution of N88S. In some embodiments, the alpha-biased IL-2 variant comprises an amino acid substitution of N88L. In some embodiments, the alpha-biased IL-2 variant comprises an amino acid substitution of N88D. In some embodiments, the alpha-biased IL-2 variant comprises an amino acid substitution of N88A. In some embodiments, the alpha-biased IL-2 variant comprises an amino acid substitution of N88E. In some embodiments, the alpha-biased IL-2 variant comprises an amino acid substitution of N88F. In some embodiments, the alpha-biased IL-2 variant comprises an amino acid substitution of N88G. In some embodiments, the alpha-biased IL-2 variant comprises an amino acid substitution of N88M. In some embodiments, the alpha-biased IL-2 variant comprises an amino acid substitution of N88R. In some embodiments, the alpha-biased IL-2 variant comprises an amino acid substitution of N88V. In some embodiments, the alpha-biased IL-2 variant comprises an amino acid substitution of N88W. In some embodiments, the alpha-biased IL-2 variant comprises an amino acid substitution of N88I. In some embodiments, the alpha-biased IL-2 variant comprises an amino acid substitution of N88Q. In some embodiments, the alpha-biased IL-2 variant comprises an amino acid substitution of N88M. In some embodiments, the alpha-biased IL-2 variant comprises an amino acid substitution of N88T.

In some embodiments, the alpha-biased IL-2 variant comprises an amino acid substitution at position N90. In some embodiments, the alpha-biased IL-2 variant comprises an amino acid substitution of N90H.

In some embodiments, the alpha-biased IL-2 variant comprises an amino acid substitution at position V91. In some embodiments, the alpha-biased IL-2 variant comprises an amino acid substitution of V91K. In some embodiments, the alpha-biased IL-2 variant comprises an amino acid substitution of V91D. In some embodiments, the alpha-biased IL-2 variant comprises an amino acid substitution of V91E. In some embodiments, the alpha-biased IL-2 variant comprises an amino acid substitution of V91G. In some embodiments, the alpha-biased IL-2 variant comprises an amino acid substitution of V91S. In some embodiments, the alpha-biased IL-2 variant comprises an amino acid substitution of V91L.

In some embodiments, the alpha-biased IL-2 variant comprises an amino acid substitution at position I92. In some embodiments, the alpha-biased IL-2 variant comprises an amino acid substitution of I92S. In some embodiments, the alpha-biased IL-2 variant comprises an amino acid substitution of I92K. In some embodiments, the alpha-biased IL-2 variant comprises an amino acid substitution of I92R. In some embodiments, the alpha-biased IL-2 variant comprises an amino acid substitution of I92L.

In some embodiments, the alpha-biased IL-2 variant comprises an amino acid substitution at position E95. In some embodiments, the alpha-biased IL-2 variant comprises an amino acid substitution of E95G. In some embodiments, the alpha-biased IL-2 variant comprises an amino acid substitution of E95Q.

In some embodiments, the alpha-biased IL-2 variant comprises an amino acid substitution at position T101. In some embodiments, the alpha-biased IL-2 variant comprises an amino acid substitution of T101R. In some embodiments, the alpha-biased IL-2 variant comprises an amino acid substitution of T101A.

In some embodiments, the alpha-biased IL-2 variant comprises an amino acid substitution at position F103. In some embodiments, the alpha-biased IL-2 variant comprises an amino acid substitution of F103S.

In some embodiments, the alpha-biased IL-2 variant comprises an amino acid substitution at position D109. In some embodiments, the alpha-biased IL-2 variant comprises an amino acid substitution of D109N.

In some embodiments, the alpha-biased IL-2 variant comprises an amino acid substitution at position I114. In some embodiments, the alpha-biased IL-2 variant comprises an amino acid substitution of I114V.

In some embodiments, the alpha-biased IL-2 variant comprises an amino acid substitution at position L118. In some embodiments, the alpha-biased IL-2 variant comprises an amino acid substitution of L118I.

In some embodiments, the alpha-biased IL-2 variant comprises an amino acid substitution at position N119. In some embodiments, the alpha-biased IL-2 variant comprises an amino acid substitution of N119D.

In some embodiments, the alpha-biased IL-2 variant comprises an amino acid substitution at position R120. In some embodiments, the alpha-biased IL-2 variant comprises an amino acid substitution of R120G. In some embodiments, the alpha-biased IL-2 variant comprises an amino acid substitution of R120D.

In some embodiments, the alpha-biased IL-2 variant comprises an amino acid substitution at position T123. In some embodiments, the alpha-biased IL-2 variant comprises an amino acid substitution of T123S. In some embodiments, the alpha-biased IL-2 variant comprises an amino acid substitution of T123A.

In some embodiments, the alpha-biased IL-2 variant comprises an amino acid substitution at position C125. In some embodiments, the alpha-biased IL-2 variant comprises an amino acid substitution of C125S. In some embodiments, the alpha-biased IL-2 variant comprises an amino acid substitution of C125A. In some embodiments, the alpha-biased IL-2 variant comprises an amino acid substitution of C125V. In some embodiments, the alpha-biased IL-2 variant comprises an amino acid substitution of C125E. In some embodiments, the alpha-biased IL-2 variant comprises an amino acid substitution of C125K. In some embodiments, the alpha-biased IL-2 variant comprises an amino acid substitution of C125H. In some embodiments, the alpha-biased IL-2 variant comprises an amino acid substitution of C125W. In some embodiments, the alpha-biased IL-2 variant comprises an amino acid substitution of C125I.

In some embodiments, the alpha-biased IL-2 variant comprises an amino acid substitution at position Q126. In some embodiments, the alpha-biased IL-2 variant comprises an amino acid substitution of Q126R. In some embodiments, the alpha-biased IL-2 variant comprises an amino acid substitution of Q126T. In some embodiments, the alpha-biased IL-2 variant comprises an amino acid substitution of Q126K. In some embodiments, the alpha-biased IL-2 variant comprises an amino acid substitution of Q126E. In some embodiments, the alpha-biased IL-2 variant comprises an amino acid substitution of Q126L. In some embodiments, the alpha-biased IL-2 variant comprises an amino acid substitution of Q126N. In some embodiments, the alpha-biased IL-2 variant comprises an amino acid substitution of Q126D. In some embodiments, the alpha-biased IL-2 variant comprises an amino acid substitution of Q126M. In some embodiments, the alpha-biased IL-2 variant comprises an amino acid substitution of Q126H. In some embodiments, the alpha-biased IL-2 variant comprises an amino acid substitution of Q126Y.

In some embodiments, the alpha-biased IL-2 variant comprises an amino acid substitution at position I128. In some embodiments, the alpha-biased IL-2 variant comprises an amino acid substitution of I128T.

In some embodiments, the alpha-biased IL-2 variant comprises an amino acid substitution at position S130. In some embodiments, the alpha-biased IL-2 variant comprises an amino acid substitution of S130T.

In some embodiments, the alpha-biased IL-2 variant comprises an amino acid substitution at position T133. In some embodiments, the alpha-biased IL-2 variant comprises an amino acid substitution of T133S. In some embodiments, the alpha-biased IL-2 variant comprises an amino acid substitution of T133N.

In some embodiments, the alpha-biased IL-2 variant comprises an amino acid substitution of H16L, H16N, V69A, Q74P, I92S, D84V, and/or S87R. In some embodiments, the alpha-biased IL-2 variant comprises an amino acid substitution of H16L. In some embodiments, the alpha-biased IL-2 variant comprises an amino acid substitution of H16N. In some embodiments, the alpha-biased IL-2 variant comprises an amino acid substitution of V69A. In some embodiments, the alpha-biased IL-2 variant comprises an amino acid substitution of Q74P. In some embodiments, the alpha-biased IL-2 variant comprises an amino acid substitution of I92S. In some embodiments, the alpha-biased IL-2 variant comprises an amino acid substitution of D84V. In some embodiments, the alpha-biased IL-2 variant comprises an amino acid substitution of S87R.

In some embodiments, the alpha-biased IL-2 variant comprises the amino acid substitutions of H16L, V69A, and Q74P. In some embodiments, the alpha-biased IL-2 variant comprises the amino acid substitutions of H16N, V69A, and Q74P. In some embodiments, the alpha-biased IL-2 variant comprises the amino acid substitutions of V69A, Q74P, and S87R. In some embodiments, the alpha-biased IL-2 variant comprises the amino acid substitutions of V69A, Q74P, and D84V. In some embodiments, the alpha-biased IL-2 variant comprises the amino acid substitutions of V69A, Q74P, and I92S.

In some embodiments, the alpha-biased IL-2 variant further comprises the amino acid substitution of C125S.

In some embodiments, the alpha-biased IL-2 variant further comprises the amino acid substitution of T3A.

In some embodiments, the alpha-biased IL-2 variant comprises the amino acid substitutions of H16L, V69A, Q74P, and C125S. In some embodiments, the alpha-biased IL-2 variant comprises the amino acid substitutions of H16N, V69A, Q74P, and C125S. In some embodiments, the alpha-biased IL-2 variant comprises the amino acid substitutions of V69A, Q74P, S87R, and C125S. In some embodiments, the alpha-biased IL-2 variant comprises the amino acid substitutions of V69A, Q74P, D84V, and C125S. In some embodiments, the alpha-biased IL-2 variant comprises the amino acid substitutions of V69A, Q74P, I92S, and C125S.

In some embodiments, the alpha-biased IL-2 variant comprises the amino acid substitutions of T3A, H16L, V69A, and Q74P. In some embodiments, the alpha-biased IL-2 variant comprises the amino acid substitutions of T3A, H16N, V69A, and Q74P. In some embodiments, the alpha-biased IL-2 variant comprises the amino acid substitutions of T3A, V69A, Q74P, and S87R. In some embodiments, the alpha-biased IL-2 variant comprises the amino acid substitutions of T3A, V69A, Q74P, and D84V. In some embodiments, the alpha-biased IL-2 variant comprises the amino acid substitutions of T3A, V69A, Q74P, and I92S.

In some embodiments, the alpha-biased IL-2 variant comprises the amino acid substitutions of T3A, H16L, V69A, Q74P, and C125S. In some embodiments, the alpha-biased IL-2 variant comprises the amino acid substitutions of T3A, H16N, V69A, Q74P, and C125S. In some embodiments, the alpha-biased IL-2 variant comprises the amino acid substitutions of T3A, V69A, Q74P, S87R, and C125S. In some embodiments, the alpha-biased IL-2 variant comprises the amino acid substitutions of T3A, V69A, Q74P, D84V, and C125S. In some embodiments, the alpha-biased IL-2 variant comprises the amino acid substitutions of T3A, V69A, Q74P, I92S, and C125S.

In some embodiments, the alpha-biased IL-2 variant comprises an amino acid sequence that is at least 90%, at least 95%, at least 98%, or at least 99% identical to SEQ ID NO: 2. In some embodiments, the alpha-biased IL-2 variant comprises an amino acid sequence that is at least 90% identical to SEQ ID NO: 2. In some embodiments, the alpha-biased IL-2 variant comprises an amino acid sequence that is at least 91% identical to SEQ ID NO: 2. In some embodiments, the alpha-biased IL-2 variant comprises an amino acid sequence that is at least 92% identical to SEQ ID NO: 2. In some embodiments, the alpha-biased IL-2 variant comprises an amino acid sequence that is at least 93% identical to SEQ ID NO: 2. In some embodiments, the alpha-biased IL-2 variant comprises an amino acid sequence that is at least 94% identical to SEQ ID NO: 2. In some embodiments, the alpha-biased IL-2 variant comprises an amino acid sequence that is at least 95% identical to SEQ ID NO: 2. In some embodiments, the alpha-biased IL-2 variant comprises an amino acid sequence that is at least 96% identical to SEQ ID NO: 2. In some embodiments, the alpha-biased IL-2 variant comprises an amino acid sequence that is at least 97% identical to SEQ ID NO: 2. In some embodiments, the alpha-biased IL-2 variant comprises an amino acid sequence that is at least 98% identical to SEQ ID NO: 2. In some embodiments, the alpha-biased IL-2 variant comprises an amino acid sequence that is at least 99% identical to SEQ ID NO: 2. In some embodiments, the alpha-biased IL-2 variant comprises an amino acid sequence of SEQ ID NO: 2.

In some embodiments, the alpha-biased IL-2 variant comprises an amino acid sequence that is at least 90%, at least 95%, at least 98%, or at least 99% identical to SEQ ID NO: 3. In some embodiments, the alpha-biased IL-2 variant comprises an amino acid sequence that is at least 90% identical to SEQ ID NO: 3. In some embodiments, the alpha-biased IL-2 variant comprises an amino acid sequence that is at least 91% identical to SEQ ID NO: 3. In some embodiments, the alpha-biased IL-2 variant comprises an amino acid sequence that is at least 92% identical to SEQ ID NO: 3. In some embodiments, the alpha-biased IL-2 variant comprises an amino acid sequence that is at least 93% identical to SEQ ID NO: 3. In some embodiments, the alpha-biased IL-2 variant comprises an amino acid sequence that is at least 94% identical to SEQ ID NO: 3. In some embodiments, the alpha-biased IL-2 variant comprises an amino acid sequence that is at least 95% identical to SEQ ID NO: 3. In some embodiments, the alpha-biased IL-2 variant comprises an amino acid sequence that is at least 96% identical to SEQ ID NO: 3. In some embodiments, the alpha-biased IL-2 variant comprises an amino acid sequence that is at least 97% identical to SEQ ID NO: 3. In some embodiments, the alpha-biased IL-2 variant comprises an amino acid sequence that is at least 98% identical to SEQ ID NO: 3. In some embodiments, the alpha-biased IL-2 variant comprises an amino acid sequence that is at least 99% identical to SEQ ID NO: 3. In some embodiments, the alpha-biased IL-2 variant comprises an amino acid sequence of SEQ ID NO: 3.

In some embodiments, the alpha-biased IL-2 variant comprises an amino acid sequence that is at least 90%, at least 95%, at least 98%, or at least 99% identical to SEQ ID NO: 4. In some embodiments, the alpha-biased IL-2 variant comprises an amino acid sequence that is at least 90% identical to SEQ ID NO: 4. In some embodiments, the alpha-biased IL-2 variant comprises an amino acid sequence that is at least 91% identical to SEQ ID NO: 4. In some embodiments, the alpha-biased IL-2 variant comprises an amino acid sequence that is at least 92% identical to SEQ ID NO: 4. In some embodiments, the alpha-biased IL-2 variant comprises an amino acid sequence that is at least 93% identical to SEQ ID NO: 4. In some embodiments, the alpha-biased IL-2 variant comprises an amino acid sequence that is at least 94% identical to SEQ ID NO: 4. In some embodiments, the alpha-biased IL-2 variant comprises an amino acid sequence that is at least 95% identical to SEQ ID NO: 4. In some embodiments, the alpha-biased IL-2 variant comprises an amino acid sequence that is at least 96% identical to SEQ ID NO: 4. In some embodiments, the alpha-biased IL-2 variant comprises an amino acid sequence that is at least 97% identical to SEQ ID NO: 4. In some embodiments, the alpha-biased IL-2 variant comprises an amino acid sequence that is at least 98% identical to SEQ ID NO: 4. In some embodiments, the alpha-biased IL-2 variant comprises an amino acid sequence that is at least 99% identical to SEQ ID NO: 4. In some embodiments, the alpha-biased IL-2 variant comprises an amino acid sequence of SEQ ID NO: 4.

In some embodiments, the alpha-biased IL-2 variant comprises an amino acid sequence that is at least 90%, at least 95%, at least 98%, or at least 99% identical to SEQ ID NO: 5. In some embodiments, the alpha-biased IL-2 variant comprises an amino acid sequence that is at least 90% identical to SEQ ID NO: 5. In some embodiments, the alpha-biased IL-2 variant comprises an amino acid sequence that is at least 91% identical to SEQ ID NO: 5. In some embodiments, the alpha-biased IL-2 variant comprises an amino acid sequence that is at least 92% identical to SEQ ID NO: 5. In some embodiments, the alpha-biased IL-2 variant comprises an amino acid sequence that is at least 93% identical to SEQ ID NO: 5. In some embodiments, the alpha-biased IL-2 variant comprises an amino acid sequence that is at least 94% identical to SEQ ID NO: 5. In some embodiments, the alpha-biased IL-2 variant comprises an amino acid sequence that is at least 95% identical to SEQ ID NO: 5. In some embodiments, the alpha-biased IL-2 variant comprises an amino acid sequence that is at least 96% identical to SEQ ID NO: 5. In some embodiments, the alpha-biased IL-2 variant comprises an amino acid sequence that is at least 97% identical to SEQ ID NO: 5. In some embodiments, the alpha-biased IL-2 variant comprises an amino acid sequence that is at least 98% identical to SEQ ID NO: 5. In some embodiments, the alpha-biased IL-2 variant comprises an amino acid sequence that is at least 99% identical to SEQ ID NO: 5. In some embodiments, the alpha-biased IL-2 variant comprises an amino acid sequence of SEQ ID NO: 5.

In some embodiments, the alpha-biased IL-2 variant comprises an amino acid sequence that is at least 90%, at least 95%, at least 98%, or at least 99% identical to SEQ ID NO: 6. In some embodiments, the alpha-biased IL-2 variant comprises an amino acid sequence that is at least 90% identical to SEQ ID NO: 6. In some embodiments, the alpha-biased IL-2 variant comprises an amino acid sequence that is at least 91% identical to SEQ ID NO: 6. In some embodiments, the alpha-biased IL-2 variant comprises an amino acid sequence that is at least 92% identical to SEQ ID NO: 6. In some embodiments, the alpha-biased IL-2 variant comprises an amino acid sequence that is at least 93% identical to SEQ ID NO: 6. In some embodiments, the alpha-biased IL-2 variant comprises an amino acid sequence that is at least 94% identical to SEQ ID NO: 6. In some embodiments, the alpha-biased IL-2 variant comprises an amino acid sequence that is at least 95% identical to SEQ ID NO: 6. In some embodiments, the alpha-biased IL-2 variant comprises an amino acid sequence that is at least 96% identical to SEQ ID NO: 6. In some embodiments, the alpha-biased IL-2 variant comprises an amino acid sequence that is at least 97% identical to SEQ ID NO: 6. In some embodiments, the alpha-biased IL-2 variant comprises an amino acid sequence that is at least 98% identical to SEQ ID NO: 6. In some embodiments, the alpha-biased IL-2 variant comprises an amino acid sequence that is at least 99% identical to SEQ ID NO: 6. In some embodiments, the alpha-biased IL-2 variant comprises an amino acid sequence of SEQ ID NO: 6.

In some embodiments, the alpha-biased IL-2 variant comprises an amino acid sequence that is at least 90%, at least 95%, at least 98%, or at least 99% identical to SEQ ID NO: 7. In some embodiments, the alpha-biased IL-2 variant comprises an amino acid sequence that is at least 90% identical to SEQ ID NO: 7. In some embodiments, the alpha-biased IL-2 variant comprises an amino acid sequence that is at least 91% identical to SEQ ID NO: 7. In some embodiments, the alpha-biased IL-2 variant comprises an amino acid sequence that is at least 92% identical to SEQ ID NO: 7. In some embodiments, the alpha-biased IL-2 variant comprises an amino acid sequence that is at least 93% identical to SEQ ID NO: 7. In some embodiments, the alpha-biased IL-2 variant comprises an amino acid sequence that is at least 94% identical to SEQ ID NO: 7. In some embodiments, the alpha-biased IL-2 variant comprises an amino acid sequence that is at least 95% identical to SEQ ID NO: 7. In some embodiments, the alpha-biased IL-2 variant comprises an amino acid sequence that is at least 96% identical to SEQ ID NO: 7. In some embodiments, the alpha-biased IL-2 variant comprises an amino acid sequence that is at least 97% identical to SEQ ID NO: 7. In some embodiments, the alpha-biased IL-2 variant comprises an amino acid sequence that is at least 98% identical to SEQ ID NO: 7. In some embodiments, the alpha-biased IL-2 variant comprises an amino acid sequence that is at least 99% identical to SEQ ID NO: 7. In some embodiments, the alpha-biased IL-2 variant comprises an amino acid sequence of SEQ ID NO: 7.

In some embodiments, the alpha-biased IL-2 variant comprises the amino acid substitutions of H16L, V69A, and Q74P, and comprises an amino acid sequence at least 95% identical to SEQ ID NO: 3. In some embodiments, the alpha-biased IL-2 variant comprises the amino acid substitutions of H16N, V69A, and Q74P, and comprises an amino acid sequence at least 95% identical to SEQ ID NO: 3. In some embodiments, the alpha-biased IL-2 variant comprises the amino acid substitutions of V69A, Q74P, and D84V, and comprises an amino acid sequence at least 95% identical to SEQ ID NO: 3. In some embodiments, the alpha-biased IL-2 variant comprises the amino acid substitutions of V69A, Q74P, and S87R, and comprises an amino acid sequence at least 95% identical to SEQ ID NO: 3. In some embodiments, the alpha-biased IL-2 variant comprises the amino acid substitutions of V69A, Q74P, and I92S, and comprises an amino acid sequence at least 95% identical to SEQ ID NO: 3.

TABLE 2
Sequences of Exemplary IL-2 Variants.

| SEQ ID NO | Description | Substitutions | Sequence |
|---|---|---|---|
| 1 | IL2 WT | None | APTSSSTKKTQLQLEHLLLDLQMILNGINNYKNPK |
| | | | LTRMLTFKFYMPKKATELKHLQCLEEELKPLEEVL |
| | | | NLAQSKNFHLRPRDLISNINVIVLELKGSETTFMC |
| | | | EYADETATIVEFLNRWITFCQSIISTLT |
| 2 | IL2 Variant | C125S | APTSSSTKKTQLQLEHLLLDLQMILNGINNYKNPK |
| | | | LTRMLTFKFYMPKKATELKHLQCLEEELKPLEEVL |
| | | | NLAQSKNFHLRPRDLISNINVIVLELKGSETTFMC |
| | | | EYADETATIVEFLNRWITFSQSIISTLT |
| 3 | IL2 Variant | V69A/Q74P | APTSSSTKKTQLQLEHLLLDLQMILNGINNYKNPK |
| | | /C125S | LTRMLTFKFYMPKKATELKHLQCLEEELKPLEEAL |
| | | | NLAPSKNFHLRPRDLISNINVIVLELKGSETTFMC |
| | | | EYADETATIVEFLNRWITFSQSIISTLT |
| 4 | IL2 Variant | H16L/V69A | APTSSSTKKTQLQLELLLLDLQMILNGINNYKNPK |
| | | /Q74P/ | LTRMLTFKFYMPKKATELKHLQCLEEELKPLEEAL |
| | | C125S | NLAPSKNFHLRPRDLISNINVIVLELKGSETTEMC |
| | | | EYADETATIVEFLNRWITFSQSIISTLT |
| 5 | IL2 Variant | H16N/V69A | APTSSSTKKTQLQLENLLLDLQMILNGINNYKNPK |
| | | /Q74P/ | LTRMLTFKFYMPKKATELKHLQCLEEELKPLEEAL |
| | | C125S | NLAPSKNFHLRPRDLISNINVIVLELKGSETTFMC |
| | | | EYADETATIVEFLNRWITFSQSIISTLT |
| 6 | IL2 Variant | V69A/Q74P | APTSSSTKKTQLQLEHLLLDLQMILNGINNYKNPK |
| | | /192S/ | LTRMLTFKFYMPKKATELKHLQCLEEELKPLEEAL |
| | | C125S | NLAPSKNFHLRPRDLISNINVSVLELKGSETTFMC |
| | | | EYADETATIVEFLNRWITFSQSIISTLT |
| 7 | IL2 Variant | T3A/V69A/ | APASSSTKKTQLQLEHLLLDLQMILNGINNYKNPK |
| | | Q74P/ | LTRMLTFKFYMPKKATELKHLQCLEEELKPLEEAL |
| | | C125S | NLAPSKNFHLRPRDLISNINVIVLELKGSETTFMC |
| | | | EYADETATIVEFLNRWITFSQSIISTLT |

In some embodiments, an alpha-biased IL-2 variant comprises amino acid substitutions of V69A and Q74P relative to SEQ ID NO: 1. In some embodiments, an alpha-biased IL-2 variant comprises amino acid substitutions of H16L, V69A and Q74P relative to SEQ ID NO: 1. In some embodiments, an alpha-biased IL-2 variant comprises amino acid substitutions of H16N, V69A and Q74P relative to SEQ ID NO: 1. In some embodiments, an alpha-biased IL-2 variant comprises amino acid substitutions of V69A, Q74P and I92S relative to SEQ ID NO: 1. In some embodiments, an alpha-biased IL-2 variant comprises amino acid substitutions of T3A, V69A and Q74P relative to SEQ ID NO: 1. In some embodiments, an alpha-biased IL-2 variant comprises amino acid substitutions of V69A, Q74P and C125S relative to SEQ ID NO: 1. In some embodiments, an alpha-biased IL-2 variant comprises amino acid substitutions of H16L, V69A, Q74P and C125S relative to SEQ ID NO: 1. In some embodiments, an alpha-biased IL-2 variant comprises amino acid substitutions of H16N, V69A, Q74P and C125S relative to SEQ ID NO: 1. In some embodiments, an alpha-biased IL-2 variant comprises amino acid substitutions of V69A, Q74P, I92S and C125S relative to SEQ ID NO: 1. In some embodiments, an alpha-biased IL-2 variant comprises amino acid substitutions of T3A, V69A Q74P and C125S relative to SEQ ID NO: 1.

In some embodiments, an alpha-biased IL-2 variant comprises amino acid substitutions of V69A and Q74P relative to SEQ ID NO: 2. In some embodiments, an alpha-biased IL-2 variant comprises amino acid substitutions of H16L, V69A and Q74P relative to SEQ ID NO: 2. In some embodiments, an alpha-biased IL-2 variant comprises amino acid substitutions of H16N, V69A and Q74P relative to SEQ ID NO: 2. In some embodiments, an alpha-biased IL-2 variant comprises amino acid substitutions of V69A, Q74P and I92S relative to SEQ ID NO: 2. In some embodiments, an alpha-biased IL-2 variant comprises amino acid substitutions of T3A, V69A and Q74P relative to SEQ ID NO: 2.

The alpha-biased IL-2 variants of the present invention may comprise any mutation well known in the art to have increased binding affinity to CD25 and/or decreased binding affinity for CD122 and/or CD132 as compared to a wild-type IL-2. In some embodiments, the alpha-biased IL-2 variant comprises one or more mutations or combinations disclosed in U.S. Published Patent Application Nos. US20190300592A1, US20240043491A1, US20220213162A1, US20240059751A1, US20110274650A1, US20050142106A1, or US20140343252A1, each of which is incorporated herein by reference. In some embodiments, the alpha-biased IL-2 variant comprises one or more mutations or combinations disclosed in International Application No. WO2016164937A2, which is incorporated herein by reference.

### Alpha-Biased IL-2 Fusion Proteins

In some embodiments, a suitable alpha-biased IL-2 variant for the present invention may be fused to a half-life extension domain for the purpose of the present invention. Half-life extension domains suitable for the present invention include, but are not limited to, an Fc domain, an albumin binding domain, a human serum albumin, lipids, and polyethylene glycols (PEGs).

In some embodiments, a suitable alpha-biased IL-2 variant is fused to an Fc domain. In some embodiments, a suitable alpha-biased IL-2 variant is fused to an albumin binding domain. In some embodiments, a suitable alpha-biased IL-2 variant is fused to a human serum albumin. In some embodiments, a suitable alpha-biased IL-2 variant is fused to one or more lipids. In some embodiments, a suitable alpha-biased IL-2 variant is fused to one or more polyethylene glycols (PEGs).

In some embodiments, the alpha-biased IL-2 fusion protein forms a dimer. In some embodiments, the alpha-biased IL-2 fusion protein forms a heterodimer. In some embodiments, the alpha-biased IL-2 fusion protein forms a homodimer.

### Fc Domain

In some embodiments, the alpha-biased IL-2 fusion protein is an alpha-biased IL-2-Fc fusion protein. In some embodiments, the half-life extension domain is an Fc domain. In some embodiments, the half-life extension domain is an Fc domain of IgG1. In some embodiments, the half-life extension domain is an Fc domain of IgG2. In some embodiments, the half-life extension domain is an Fc domain of IgG3. In some embodiments, the half-life extension domain is an Fc domain of IgG4.

In some embodiments, an Fc domain is a modified Fc domain comprising one or more mutations. In some embodiments, the Fc domain is engineered to reduce effector function. In some embodiments, the Fc domain is engineered to further increase half-life.

In some embodiments, the Fc domain comprises one or more (e.g., 2, 3, 4, 5, 6, or 7) mutations in residues chosen from T256, H285, N286, T307, Q311, N315, or A378. In some embodiments, the Fc domain comprises one or more (e.g., 2, 3, 4, 5, 6, or 7) mutations chosen from T256D, H285N, N286D, T307Q, Q311V, N315D, or A378V.

In some embodiments, the Fc domain comprises a half-life enhancing mutation, a mutation that is capable of disrupting an Fc effector function, or both. In some embodiments, the Fc domain comprises one or more mutations or combinations of mutations described herein, e.g., chosen from M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in some embodiments, the Fc domain comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., N297G, L234A/L235A (also known as “LALA” mutation), L234A/L235A/P329G (also known as “LALAPG” mutation), or (c) both (a) and (b).

In some embodiments, the Fc domain comprises mutations T256D/Q311V/A378V and a mutation or a combination of mutations capable of disrupting an Fc effector function, e.g., N297G. In some embodiments, the Fc domain comprises mutations H285N/T307Q/N315D and a mutation or a combination of mutations capable of disrupting an Fc effector function, e.g., N297G. In some embodiments, the Fc domain comprises mutations H285D/T307Q/A378V and a mutation or a combination of mutations capable of disrupting an Fc effector function, e.g., N297G. In some embodiments, the Fc domain comprises mutations T307Q/Q311V/A378V and a mutation or a combination of mutations capable of disrupting an Fc effector function, e.g., N297G. In some embodiments, the Fc domain comprises mutations T256D/N286D/T307R/Q311V/A378V and a mutation or a combination of mutations capable of disrupting an Fc effector function, e.g., N297G. In some embodiments, the Fc domain comprises mutations T256D/T307R/Q311V and a mutation or a combination of mutations capable of disrupting an Fc effector function, e.g., N297G.

In some embodiments, the Fc domain comprises mutations T256D/Q311V/A378V and a mutation or a combination of mutations capable of disrupting an Fc effector function, e.g., L234A/L235A. In some embodiments, the Fc domain comprises mutations H285N/T307Q/N315D and a mutation or a combination of mutations capable of disrupting an Fc effector function, e.g., L234A/L235A. In some embodiments, the Fc domain comprises mutations H285D/T307Q/A378V and a mutation or a combination of mutations capable of disrupting an Fc effector function, e.g., L234A/L235A. In some embodiments, the Fc domain comprises mutations T307Q/Q311V/A378V and a mutation or a combination of mutations capable of disrupting an Fc effector function, e.g., L234A/L235A. In some embodiments, the Fc domain comprises mutations T256D/N286D/T307R/Q311V/A378V and a mutation or a combination of mutations capable of disrupting an Fc effector function, e.g., L234A/L235A. In some embodiments, the Fc domain comprises mutations T256D/T307R/Q311V and a mutation or a combination of mutations capable of disrupting an Fc effector function, e.g., L234A/L235A.

In some embodiments, the Fc domain comprises mutations T256D/Q311V/A378V and a mutation or a combination of mutations capable of disrupting an Fc effector function, e.g., L234A/L235A/P329G. In some embodiments, the Fc domain comprises mutations H285N/T307Q/N315D and a mutation or a combination of mutations capable of disrupting an Fc effector function, e.g., L234A/L235A/P329G. In some embodiments, the Fc domain comprises mutations H285D/T307Q/A378V and a mutation or a combination of mutations capable of disrupting an Fc effector function, e.g., L234A/L235A/P329G. In some embodiments, the Fc domain comprises mutations T307Q/Q311V/A378V and a mutation or a combination of mutations capable of disrupting an Fc effector function, e.g., L234A/L235A/P329G. In some embodiments, the Fc domain comprises mutations T256D/N286D/T307R/Q311V/A378V and a mutation or a combination of mutations capable of disrupting an Fc effector function, e.g., L234A/L235A/P329G. In some embodiments, the Fc domain comprises mutations T256D/T307R/Q311V and a mutation or a combination of mutations capable of disrupting an Fc effector function, e.g., L234A/L235A/P329G.

In some embodiments the Fc domain comprises the N297G mutation. In some embodiments the Fc domain comprises the L234A/L235A mutations. In some embodiments, the Fc domain comprises the L234A/L235A/P329G mutations.

In some embodiments the Fc domain comprises the Fc domain of human IgG1, e.g., human IgG1 m3 allotype. In some embodiments, the Fc domain comprises the mutation N297G. In some embodiments, the Fc domain comprises the Fc domain of human IgG1 allotype m3, human IgG1 allotype m3 comprising the mutation N297G and/or other mutations of the Fc domain of human IgG1 allotype m3, or a fragment thereof.

In some embodiments, the Fc domain comprises an amino acid sequence at least 90%, at least 95%, at least 98%, at least 99% identical to an amino acid sequence selected from Table 3.

In some embodiments, the Fc domain comprises an amino acid sequence at least 90% identical to SEQ ID NO: 8. In some embodiments, the Fc domain comprises an amino acid sequence at least 95% identical to SEQ ID NO: 8. In some embodiments, the Fc domain comprises an amino acid sequence at least 98% identical to SEQ ID NO: 8. In some embodiments, the Fc domain comprises an amino acid sequence at least 99% identical to SEQ ID NO: 8. In some embodiments, the Fc domain comprises an amino acid sequence identical to SEQ ID NO: 8.

In some embodiments, the Fc domain comprises an amino acid sequence at least 90% identical to SEQ ID NO: 9. In some embodiments, the Fc domain comprises an amino acid sequence at least 95% identical to SEQ ID NO: 9. In some embodiments, the Fc domain comprises an amino acid sequence at least 98% identical to SEQ ID NO: 9. In some embodiments, the Fc domain comprises an amino acid sequence at least 99% identical to SEQ ID NO: 9. In some embodiments, the Fc domain comprises an amino acid sequence identical to SEQ ID NO: 9.

In some embodiments, the Fc domain comprises an amino acid sequence at least 90% identical to SEQ ID NO: 10. In some embodiments, the Fc domain comprises an amino acid sequence at least 95% identical to SEQ ID NO: 10. In some embodiments, the Fc domain comprises an amino acid sequence at least 98% identical to SEQ ID NO: 10. In some embodiments, the Fc domain comprises an amino acid sequence at least 99% identical to SEQ ID NO: 10. In some embodiments, the Fc domain comprises an amino acid sequence identical to SEQ ID NO: 10.

In some embodiments, the Fc domain comprises an amino acid sequence at least 90% identical to SEQ ID NO: 11. In some embodiments, the Fc domain comprises an amino acid sequence at least 95% identical to SEQ ID NO: 11. In some embodiments, the Fc domain comprises an amino acid sequence at least 98% identical to SEQ ID NO: 11. In some embodiments, the Fc domain comprises an amino acid sequence at least 99% identical to SEQ ID NO: 11. In some embodiments, the Fc domain comprises an amino acid sequence identical to SEQ ID NO: 11.

TABLE 3
Sequences of Exemplary Fc Domains.

| SEQ ID NO | Description | Substitutions | Sequence |
|---|---|---|---|
| 8 | IgG1 Fc | | DKTHTCPPCPAPELLGGPSVFLFPPKPKDILMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALPAPIEKTISKAKGQPREPQVYTLPPSRDELTK |
| | | | NQVSLTCLVKGFYPSDIAVEWESNGQPENNYKITP |
| | | | PVLDSDGSFFLYSKLTVDKSRWQQGNVFSCSVMHE |
| | | | ALHNHYTQKSLSLSPGK |
| 9 | IgG1 Fc | N297G | DKTHTCPPCPAPELLGGPSVFLFPPKPKDTLMISR |
| | | | TPEVTCVVVDVSHEDPEVKFNWYVDGVEVHNAKTK |
| | | | PREEQYGSTYRVVSVLIVLHQDWLNGKEYKCKVSN |
| | | | KALPAPIEKTISKAKGQPREPQVYTLPPSRDELTK |
| | | | NQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK |
| 10 | IgG1 Fc m3 | | DKTHTCPPCPAPELLGGPSVFLFPPKPKDTLMISR |
| | allotype | | TPEVTCVVVDVSHEDPEVKFNWYVDGVEVHNAKTK |
| | | | PREEQYNSTYRVVSVLIVLHQDWLNGKEYKCKVSN |
| | | | KALPAPIEKTISKAKGQPREPQVYTLPPSREEMTK |
| | | | NQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK |
| 11 | IgG1 Fc m3 | N297G | DKTHTCPPCPAPELLGGPSVFLFPPKPKDTLMISR |
| | allotype | | TPEVTCVVVDVSHEDPEVKFNWYVDGVEVHNAKTK |
| | | | PREEQYGSTYRVVSVLTVLHQDWLNGKEYKCKVSN |
| | | | KALPAPIEKTISKAKGQPREPQVYTLPPSREEMTK |
| | | | NQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK |

Any of the mutations in the Fc domain that extend half-life described herein can be used in combination with any Fc mutation capable of enhancing or disrupting an Fc effector function. Other exemplary Fc mutations are described, e.g., in International Application Publication No. WO2018/052556, U.S. Application Publication No. US2018/0037634, and Booth et al. MAbs. 2018; 10(7): 1098-1110, the contents of which are incorporated by reference in their entirety.

### Other Half-Life Extension Domains

In some embodiments, the half-life extension domain comprises an albumin binding domain, a human serum albumin, one or more lipids, one or more polyethylene glycols (PEGs), or any other half-life extending polypeptide or polymer known in the art.

In some embodiments, the half-life extension domain comprises a serum protein binding moiety. In some embodiments, the half-life extension domain comprises human serum albumin. In some embodiments, the half-life extension domain comprises one or more lipids. In some embodiments, the half-life extension domain comprises one or more half-life extending polymers. In some embodiments, the half-life extension domain comprises one or more PEGs. In some embodiments, the half-life extension domain comprises a half-life extending polypeptide.

### Linkers

In some embodiments, the alpha-biased IL-2 variant is fused to the half-life extension domain via a linker. In some embodiments, the alpha-biased IL-2 variant is fused to the half-life extension domain via a linker such that the fusion protein comprises, from the 5′ end to the 3′ end, the alpha-biased IL-2 variant-linker-half-life extension domain. In some embodiments, the fusion protein comprises, from the 5′ end to the 3′ end, the half-life extension domain-linker-alpha-biased Il-2 variant.

In some embodiments, the linker is a polypeptide linker. In some embodiments, the linker is a flexible linker. In some embodiments, the linker is a rigid linker. In some embodiments, the linker is a cleavable linker. In some embodiments, the linker comprises an immunoglobulin hinge region or portion thereof.

In some embodiments, the linker is a Gly-Ser linker. In some embodiments, the linker comprises (G4S)ₙ, wherein n is 1, 2, 3, 4, 5, 6, or more. In some embodiments, the linker comprises (G4S)₁ (SEQ ID NO: 12). In some embodiments, the linker comprises (G4S)₂. In some embodiments, the linker comprises (G4S)₃. In some embodiments, the linker comprises (G4S)₄. In some embodiments, the linker comprises (G4S)₅. In some embodiments, the linker comprises (G4S)₆.

In some embodiments, the linker comprises a non-GS linker. In some embodiments, the linker comprises (GGGSA)ₙ, wherein n is 1, 2, 3, 4, 5, or more. In some embodiments, the linker comprises (GGGSA)₁ (SEQ ID NO: 14). In some embodiments, the linker comprises (GGGSA)₂. In some embodiments, the linker comprises (GGGSA)₃. In some embodiments, the linker comprises (GGGSA)₄. In some embodiments, the linker comprises (GGGSA)₅. In some embodiments, the linker comprises (GGGSA)₆.

In some embodiments, the linker comprises a rigid linker. In some embodiments, the linker comprises a proline-rich linker. In some embodiments, the linker comprises an α-helical rigid linker. In some embodiments, the linker comprises A(EAAAK)ₙA, wherein n is 1, 2, 3, 4, 5, 6, or more. In some embodiments, the linker comprises A(EAAAK)₁A (SEQ ID NO: 15). In some embodiments, the linker comprises A(EAAAK)₁A. In some embodiments, the linker comprises A(EAAAK)₂A. In some embodiments, the linker comprises A(EAAAK)₃A. In some embodiments, the linker comprises A(EAAAK)₄A. In some embodiments, the linker comprises A(EAAAK)₅A. In some embodiments, the linker comprises A(EAAAK)₆A.

In some embodiments, the linker comprises a sequence selected from Table 4. In some embodiments, the linker comprises an amino acid sequence of SEQ ID NO: 12. In some embodiments, the linker comprises an amino acid sequence of SEQ ID NO: 13. In some embodiments, the linker comprises an amino acid sequence of SEQ ID NO: 14. In some embodiments, the linker comprises an amino acid sequence of SEQ ID NO: 15. In some embodiments, the linker comprises an amino acid sequence of SEQ ID NO: 16. In some embodiments, the linker comprises an amino acid sequence of SEQ ID NO: 17. In some embodiments, the linker comprises an amino acid sequence of SEQ ID NO: 18. In some embodiments, the linker comprises an amino acid sequence of SEQ ID NO: 19. In some embodiments, the linker comprises an amino acid sequence of SEQ ID NO: 20. In some embodiments, the linker comprises an amino acid sequence of SEQ ID NO: 21. In some embodiments, the linker comprises an amino acid sequence of SEQ ID NO: 22.

TABLE 4
Sequences of Exemplary Linkers.

| SEQ ID NO | Description | Sequence |
|---|---|---|
| 12 | (G₄S)₁ | GGGGS |
| 13 | (G₄S)₄ linker | GGGGSGGGGSGGGGSGGGGS |
| 14 | (GGGSA)₁ | GGGSA |
| 15 | A(EAAAK)₁A | AEAAAKA |
| 16 | Linker 1 | AGSGGSGGSGGSPVPSTPPT |
| | | NSSSTPPTPSPSASGS |
| 17 | Linker 2 | AGSGGSGGSGGSPVPSTPPT |
| | | PSPSTPPTPSPSGGSGNSSG |
| | | SGGS |
| 18 | Linker 3 | AGSGNSSGSGGSGGSGNSSG |
| | | SGGSPVPSTPPTPSPSTPP |
| | | TPSPSASGS |
| 19 | Linker 4 | AEAAAKEAAAKEAAAKEAAAKAGS |
| 20 | Linker 5 | GTTPNPPASSSTTGSSTPTNPPAGS |
| 21 | Linker 6 | AGSPGAGNGGNNGGNPPPPTT |
| | | TTSSAPATTTTASAGS |
| 22 | Linker 7 | GGGSAGGGSAGGGSAGGGSAGS |

### Exemplary Alpha-Biased IL-2 Fusion Proteins Comprising Alpha-Biased IL-2 Variants

In some embodiments, the alpha-biased IL-2 fusion protein comprises an amino acid sequence at least 80% identical to any one of the sequences in Table 5. In some embodiments, the alpha-biased IL-2 fusion protein comprises an amino acid sequence at least 85% identical to any one of the sequences in Table 5. In some embodiments, the alpha-biased IL-2 fusion protein comprises an amino acid sequence at least 90% identical to any one of the sequences in Table 5. In some embodiments, the alpha-biased IL-2 fusion protein comprises an amino acid sequence at least 95% identical to any one of the sequences in Table 5. In some embodiments, the alpha-biased IL-2 fusion protein comprises an amino acid sequence at least 98% identical to any one of the sequences in Table 5. In some embodiments, the alpha-biased IL-2 fusion protein comprises an amino acid sequence at least 99% identical to any one of the sequences in Table 5. In some embodiments, the alpha-biased IL-2 fusion protein comprises an amino acid sequence identical to any one of the sequences in Table 5.

In some embodiments, the alpha-biased IL-2 fusion protein comprises an amino acid sequence that is at least 90%, at least 95%, at least 98%, or at least 99% identical to SEQ ID NO: 20. In some embodiments, the Alpha-biased IL-2 fusion protein comprises an amino acid sequence that is at least 90% identical to SEQ ID NO: 20. In some embodiments, the Alpha-biased IL-2 fusion protein comprises an amino acid sequence that is at least 95% identical to SEQ ID NO: 20. In some embodiments, the Alpha-biased IL-2 fusion protein comprises an amino acid sequence that is at least 98% identical to SEQ ID NO: 20. In some embodiments, the Alpha-biased IL-2 fusion protein comprises an amino acid sequence that is at least 99% identical to SEQ ID NO: 20. In some embodiments, the Alpha-biased IL-2 fusion protein comprises an amino acid sequence that is identical to SEQ ID NO: 20.

In some embodiments, the alpha-biased IL-2 fusion protein comprises an amino acid sequence that is at least 80%, at least 90%, at least 95%, at least 98%, or at least 99% identical to SEQ ID NO: 23. In some embodiments, the alpha-biased IL-2 fusion protein comprises an amino acid sequence that is at least 80% identical to SEQ ID NO: 23. In some embodiments, the alpha-biased IL-2 fusion protein comprises an amino acid sequence that is at least 83% identical to SEQ ID NO: 23. In some embodiments, the alpha-biased IL-2 fusion protein comprises an amino acid sequence that is at least 85% identical to SEQ ID NO: 23. In some embodiments, the alpha-biased IL-2 fusion protein comprises an amino acid sequence that is at least 87% identical to SEQ ID NO: 23. In some embodiments, the alpha-biased IL-2 fusion protein comprises an amino acid sequence that is at least 90% identical to SEQ ID NO: 23. In some embodiments, the alpha-biased IL-2 fusion protein comprises an amino acid sequence that is at least 91% identical to SEQ ID NO: 23. In some embodiments, the alpha-biased IL-2 fusion protein comprises an amino acid sequence that is at least 92% identical to SEQ ID NO: 23. In some embodiments, the alpha-biased IL-2 fusion protein comprises an amino acid sequence that is at least 93% identical to SEQ ID NO: 23. In some embodiments, the alpha-biased IL-2 fusion protein comprises an amino acid sequence that is at least 94% identical to SEQ ID NO: 23. In some embodiments, the alpha-biased IL-2 fusion protein comprises an amino acid sequence that is at least 95% identical to SEQ ID NO: 23. In some embodiments, the alpha-biased IL-2 fusion protein comprises an amino acid sequence that is at least 96% identical to SEQ ID NO: 23. In some embodiments, the alpha-biased IL-2 fusion protein comprises an amino acid sequence that is at least 97% identical to SEQ ID NO: 23. In some embodiments, the alpha-biased IL-2 fusion protein comprises an amino acid sequence that is at least 98% identical to SEQ ID NO: 23. In some embodiments, the alpha-biased IL-2 fusion protein comprises an amino acid sequence that is at least 99% identical to SEQ ID NO: 23. In some embodiments, the alpha-biased IL-2 fusion protein comprises an amino acid sequence that is identical to SEQ ID NO: 23.

In some embodiments, the alpha-biased IL-2 fusion protein comprises an amino acid sequence that is at least 80%, at least 90%, at least 95%, at least 98%, or at least 99% identical to SEQ ID NO: 24. In some embodiments, the alpha-biased IL-2 fusion protein comprises an amino acid sequence that is at least 80% identical to SEQ ID NO: 24. In some embodiments, the alpha-biased IL-2 fusion protein comprises an amino acid sequence that is at least 83% identical to SEQ ID NO: 24. In some embodiments, the alpha-biased IL-2 fusion protein comprises an amino acid sequence that is at least 85% identical to SEQ ID NO: 24. In some embodiments, the alpha-biased IL-2 fusion protein comprises an amino acid sequence that is at least 87% identical to SEQ ID NO: 24. In some embodiments, the alpha-biased IL-2 fusion protein comprises an amino acid sequence that is at least 90% identical to SEQ ID NO: 24. In some embodiments, the alpha-biased IL-2 fusion protein comprises an amino acid sequence that is at least 91% identical to SEQ ID NO: 24. In some embodiments, the alpha-biased IL-2 fusion protein comprises an amino acid sequence that is at least 92% identical to SEQ ID NO: 24. In some embodiments, the alpha-biased IL-2 fusion protein comprises an amino acid sequence that is at least 93% identical to SEQ ID NO: 24. In some embodiments, the alpha-biased IL-2 fusion protein comprises an amino acid sequence that is at least 94% identical to SEQ ID NO: 24. In some embodiments, the alpha-biased IL-2 fusion protein comprises an amino acid sequence that is at least 95% identical to SEQ ID NO: 24. In some embodiments, the alpha-biased IL-2 fusion protein comprises an amino acid sequence that is at least 96% identical to SEQ ID NO: 24. In some embodiments, the alpha-biased IL-2 fusion protein comprises an amino acid sequence that is at least 97% identical to SEQ ID NO: 24. In some embodiments, the alpha-biased IL-2 fusion protein comprises an amino acid sequence that is at least 98% identical to SEQ ID NO: 24. In some embodiments, the alpha-biased IL-2 fusion protein comprises an amino acid sequence that is at least 99% identical to SEQ ID NO: 24. In some embodiments, the alpha-biased IL-2 fusion protein comprises an amino acid sequence that is identical to SEQ ID NO: 24.

In some embodiments, the alpha-biased IL-2 fusion protein comprises an amino acid sequence that is at least 80%, at least 90%, at least 95%, at least 98%, or at least 99% identical to SEQ ID NO: 25. In some embodiments, the alpha-biased IL-2 fusion protein comprises an amino acid sequence that is at least 80% identical to SEQ ID NO: 25. In some embodiments, the alpha-biased IL-2 fusion protein comprises an amino acid sequence that is at least 83% identical to SEQ ID NO: 25. In some embodiments, the alpha-biased IL-2 fusion protein comprises an amino acid sequence that is at least 85% identical to SEQ ID NO: 25. In some embodiments, the alpha-biased IL-2 fusion protein comprises an amino acid sequence that is at least 87% identical to SEQ ID NO: 25. In some embodiments, the alpha-biased IL-2 fusion protein comprises an amino acid sequence that is at least 90% identical to SEQ ID NO: 25. In some embodiments, the alpha-biased IL-2 fusion protein comprises an amino acid sequence that is at least 91% identical to SEQ ID NO: 25. In some embodiments, the alpha-biased IL-2 fusion protein comprises an amino acid sequence that is at least 92% identical to SEQ ID NO: 25. In some embodiments, the alpha-biased IL-2 fusion protein comprises an amino acid sequence that is at least 93% identical to SEQ ID NO: 25. In some embodiments, the alpha-biased IL-2 fusion protein comprises an amino acid sequence that is at least 94% identical to SEQ ID NO: 25. In some embodiments, the alpha-biased IL-2 fusion protein comprises an amino acid sequence that is at least 95% identical to SEQ ID NO: 25. In some embodiments, the alpha-biased IL-2 fusion protein comprises an amino acid sequence that is at least 96% identical to SEQ ID NO: 25. In some embodiments, the alpha-biased IL-2 fusion protein comprises an amino acid sequence that is at least 97% identical to SEQ ID NO: 25. In some embodiments, the alpha-biased IL-2 fusion protein comprises an amino acid sequence that is at least 98% identical to SEQ ID NO: 25. In some embodiments, the alpha-biased IL-2 fusion protein comprises an amino acid sequence that is at least 99% identical to SEQ ID NO: 25. In some embodiments, the alpha-biased IL-2 fusion protein comprises an amino acid sequence that is identical to SEQ ID NO: 25.

In some embodiments, the alpha-biased IL-2 fusion protein comprises an amino acid sequence that is at least 80%, at least 90%, at least 95%, at least 98%, or at least 99% identical to SEQ ID NO: 26. In some embodiments, the alpha-biased IL-2 fusion protein comprises an amino acid sequence that is at least 80% identical to SEQ ID NO: 26. In some embodiments, the alpha-biased IL-2 fusion protein comprises an amino acid sequence that is at least 83% identical to SEQ ID NO: 26. In some embodiments, the alpha-biased IL-2 fusion protein comprises an amino acid sequence that is at least 85% identical to SEQ ID NO: 26. In some embodiments, the alpha-biased IL-2 fusion protein comprises an amino acid sequence that is at least 87% identical to SEQ ID NO: 26. In some embodiments, the alpha-biased IL-2 fusion protein comprises an amino acid sequence that is at least 90% identical to SEQ ID NO: 26. In some embodiments, the alpha-biased IL-2 fusion protein comprises an amino acid sequence that is at least 91% identical to SEQ ID NO: 26. In some embodiments, the alpha-biased IL-2 fusion protein comprises an amino acid sequence that is at least 92% identical to SEQ ID NO: 26. In some embodiments, the alpha-biased IL-2 fusion protein comprises an amino acid sequence that is at least 93% identical to SEQ ID NO: 26. In some embodiments, the alpha-biased IL-2 fusion protein comprises an amino acid sequence that is at least 94% identical to SEQ ID NO: 26. In some embodiments, the alpha-biased IL-2 fusion protein comprises an amino acid sequence that is at least 95% identical to SEQ ID NO: 26. In some embodiments, the alpha-biased IL-2 fusion protein comprises an amino acid sequence that is at least 96% identical to SEQ ID NO: 26. In some embodiments, the alpha-biased IL-2 fusion protein comprises an amino acid sequence that is at least 97% identical to SEQ ID NO: 26. In some embodiments, the alpha-biased IL-2 fusion protein comprises an amino acid sequence that is at least 98% identical to SEQ ID NO: 26. In some embodiments, the alpha-biased IL-2 fusion protein comprises an amino acid sequence that is at least 99% identical to SEQ ID NO: 26. In some embodiments, the alpha-biased IL-2 fusion protein comprises an amino acid sequence that is identical to SEQ ID NO: 26.

In some embodiments, the alpha-biased IL-2 fusion protein comprises an amino acid sequence that is at least 80%, at least 90%, at least 95%, at least 98%, or at least 99% identical to SEQ ID NO: 27. In some embodiments, the alpha-biased IL-2 fusion protein comprises an amino acid sequence that is at least 80% identical to SEQ ID NO: 27. In some embodiments, the alpha-biased IL-2 fusion protein comprises an amino acid sequence that is at least 83% identical to SEQ ID NO: 27. In some embodiments, the alpha-biased IL-2 fusion protein comprises an amino acid sequence that is at least 85% identical to SEQ ID NO: 27. In some embodiments, the alpha-biased IL-2 fusion protein comprises an amino acid sequence that is at least 87% identical to SEQ ID NO: 27. In some embodiments, the alpha-biased IL-2 fusion protein comprises an amino acid sequence that is at least 90% identical to SEQ ID NO: 27. In some embodiments, the alpha-biased IL-2 fusion protein comprises an amino acid sequence that is at least 91% identical to SEQ ID NO: 27. In some embodiments, the alpha-biased IL-2 fusion protein comprises an amino acid sequence that is at least 92% identical to SEQ ID NO: 27. In some embodiments, the alpha-biased IL-2 fusion protein comprises an amino acid sequence that is at least 93% identical to SEQ ID NO: 27. In some embodiments, the alpha-biased IL-2 fusion protein comprises an amino acid sequence that is at least 94% identical to SEQ ID NO: 27. In some embodiments, the alpha-biased IL-2 fusion protein comprises an amino acid sequence that is at least 95% identical to SEQ ID NO: 27. In some embodiments, the alpha-biased IL-2 fusion protein comprises an amino acid sequence that is at least 96% identical to SEQ ID NO: 27. In some embodiments, the alpha-biased IL-2 fusion protein comprises an amino acid sequence that is at least 97% identical to SEQ ID NO: 27. In some embodiments, the alpha-biased IL-2 fusion protein comprises an amino acid sequence that is at least 98% identical to SEQ ID NO: 27. In some embodiments, the alpha-biased IL-2 fusion protein comprises an amino acid sequence that is at least 99% identical to SEQ ID NO: 27. In some embodiments, the alpha-biased IL-2 fusion protein comprises an amino acid sequence that is identical to SEQ ID NO: 27.

In some embodiments, the alpha-biased IL-2 variant comprises the amino acid substitutions of H16L, V69A, and Q74P, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of H16N, V69A, and Q74P, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and D84V, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and S87R, and the alpha-biased IL-2 variant fused to the Fc domain comprises an amino acid sequence at least 95% identical to SEQ ID NO: 26. In some embodiments, the alpha-biased IL-2 variant comprises the amino acid substitutions of V69A, Q74P, and I92S, and the alpha-biased IL-2 variant fused to the Fc domain comprises an amino acid sequence at least 95% identical to SEQ ID NO: 26.

TABLE 5
Sequences of Exemplary Alpha-Biased IL-2 Agents

| SEQ ID NO | Description | Sequence |
|---|---|---|
| 23 | IL2 Variant | APTSSSTKKTQLQLEHLLLDLQMILNGINNYKNPK |
| | (C125S) | LTRMLTFKFYMPKKATELKHLQCLEEELKPLEEVL |
| | fused to IgG1 Fc (N297G) | NLAQSKNFHLRPRDLISNINVIVLELKGSETTFMC |
| | | EYADETATIVEFLNRWITFSQSIISTLIGGGGSGG |
| | | GGSGGGGSGGGGSDKTHTCPPCPAPELLGGPSVFL |
| | | FPPKPKDTLMISRTPEVTCVVVDVSHEDPEVKENW |
| | | YVDGVEVHNAKTKPREEQYGSTYRVVSVLTVLHQD |
| | | WLNGKEYKCKVSNKALPAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 24 | IL-2 Variant | APTSSSTKKTQLQLEHLLLDLQMILNGINNYKNPK |
| | (V69A/Q74P/C125S) | LTRMLTFKFYMPKKATELKHLQCLEEELKPLEEAL |
| | fused to IgG1 Fc (N297G) | NLAPSKNFHLRPRDLISNINVIVLELKGSETTFMC |
| | | EYADETATIVEFLNRWITFSQSIISTLIGGGGSGG |
| | | GGSGGGGSGGGGSDKTHTCPPCPAPELLGGPSVEL |
| | | FPPKPKDTLMISRTPEVTCVVVDVSHEDPEVKENW |
| | | YVDGVEVHNAKTKPREEQYGSTYRVVSVLTVLHQD |
| | | WLNGKEYKCKVSNKALPAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 25 | IL2 Variant | APTSSSTKKTQLQLELLLLDLQMILNGINNYKNPK |
| | (H16L/V69A/Q74P/C125S) | LTRMLTFKFYMPKKATELKHLQCLEEELKPLEEAL |
| | fused to IgG1 Fc (N297G) | NLAPSKNFHLRPRDLISNINVIVLELKGSETTFMC |
| | | EYADETATIVEFLNRWITFSQSIISTLTGGGGSGG |
| | | GGSGGGGSGGGGSDKTHTCPPCPAPELLGGPSVFL |
| | | FPPKPKDTLMISRTPEVTCVVVDVSHEDPEVKENW |
| | | YVDGVEVHNAKTKPREEQYGSTYRVVSVLIVLHQD |
| | | WLNGKEYKCKVSNKALPAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 26 | IL2 Variant | APTSSSTKKTQLQLEHLLLDLQMILNGINNYKNPK |
| | (V69A/Q74P/C125S) | LTRMLTFKFYMPKKATELKHLQCLEEELKPLEEAL |
| | fused to IgG1 Fc (N297G) | NLAPSKNFHLRPRDLISNINVIVLELKGSETTFMC |
| | allotype m3 | EYADETATIVEFLNRWITFSQSIISTLTGGGGSGG |
| | | GGSGGGGSGGGGSDKTHTCPPCPAPELLGGPSVFL |
| | | FPPKPKDTLMISRTPEVTCVVVDVSHEDPEVKENW |
| | | YVDGVEVHNAKTKPREEQYGSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 27 | IL2 Variant | APTSSSTKKTQLQLELLLLDLQMILNGINNYKNPK |
| | (H16L/V69A/Q74P/C125S) | LTRMLTFKFYMPKKATELKHLQCLEEELKPLEEAL |
| | fused to IgG1 Fc (N297G) | NLAPSKNFHLRPRDLISNINVIVLELKGSETTFMC |
| | allotype m3 | EYADETATIVEFLNRWITFSQSIISTLTGGGGSGG |
| | | GGSGGGGSGGGGSDKTHTCPPCPAPELLGGPSVFL |
| | | FPPKPKDTLMISRTPEVTCVVVDVSHEDPEVKENW |
| | | YVDGVEVHNAKTKPREEQYGSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |

### Use of Alpha-Biased IL-2 Variants for Kidney Transplant

Any use of, or therapy comprising, an alpha-biased IL-2 variant described herein extends to the use of an alpha-biased IL-2 fusion protein, or a fusion protein comprising an alpha-biased IL-2 variant and a half-life extension domain. Said uses also apply to the pharmaceutical compositions described herein.

The present invention provides, among other things, a method of treating kidney transplant rejection in a recipient receiving a kidney transplant.

In one aspect, the present invention provides, among other things, a method of prophylaxis for kidney rejection in a recipient receiving a kidney transplant by administering to the recipient an alpha-biased IL-2 variant described herein. In some embodiments, the method of prophylaxis for kidney rejection comprises administering to the recipient an alpha-biased IL-2 variant in conjunction with the kidney transplant. In some embodiments the method of prophylaxis for kidney rejection comprises treating the kidney rejection.

In one aspect, the present invention provides, among other things, a method of improving kidney transplant in a recipient receiving a kidney transplant by administering to the recipient an alpha-biased IL-2 variant in conjunction with the kidney transplantation. In some embodiments, the alpha-biased IL-2 variant is administered in a therapeutically effective amount. In some embodiments, the improvement in the kidney transplant results in prolonged stability of the kidney transplant, survival of the recipient, and/or reduced rejection symptoms as compared to a control.

In some embodiments, the improvement in the kidney transplant results in prolonged stability of the kidney transplant, survival of the recipient, and reduced rejection symptoms as compared to a control.

In some embodiments, the improvement in the kidney transplant results in prolonged stability of the kidney transplant as compared to a control. In some embodiments, the improvement in the kidney transplant results in the survival of the recipient as compared to a control. In some embodiments, the improvement in the kidney transplant results in reduced rejection symptoms as compared to a control. In some embodiments, the improvement in the kidney transplant results in prolonged stability of the kidney transplant and survival of the recipient. In some embodiments, the improvement in the kidney transplant results in prolonged stability of the kidney transplant and reduced rejection symptoms as compared to a control. In some embodiments, the improvement in the kidney transplant results in survival of the recipient and reduced rejection symptoms as compared to a control.

In some embodiments, the control is historical data. In some embodiments, the control is a comparable recipient without the administration of the alpha-biased IL-2 variant. In some embodiments, the control is a comparable recipient receiving a standard of care. In some embodiments, the control is a comparable recipient receiving a standard of care without the administration of the alpha-biased IL-2 variant.

In some embodiments, the standard of care comprises induction phase and maintenance phase. In some embodiments, the standard of care comprises induction phase. In some embodiments, the standard of care comprises maintenance phase.

In some embodiments, the induction phase comprises administration of rabbit antithymocyte globulin (rATG).

In some embodiments, the maintenance phase comprises administration of one or more immunosuppressive agents. In some embodiments, the one or more immunosuppressive agents comprise calcineurin inhibitors (CNI), mammalian target of rapamycin (mTOR) inhibitors, antiproliferative agents, and/or corticosteroids. In some embodiments, the one or more immunosuppressive agents comprise CNIs. In some embodiments, the one or more immunosuppressive agents comprise mTOR inhibitors. In some embodiments, the one or more immunosuppressive agents comprise antiproliferative agents. In some embodiments, the one or more immunosuppressive agents comprise corticosteroids.

In some embodiments, the one or more immunosuppressive agents comprise CNIs and mTOR inhibitors. In some embodiments, the one or more immunosuppressive agents comprise CNIs and antiproliferative agents. In some embodiments, the one or more immunosuppressive agents comprise CNIs and corticosteroids. In some embodiments, the one or more immunosuppressive agents comprise mTOR inhibitors and antiproliferative agents. In some embodiments, the one or more immunosuppressive agents comprise mTOR inhibitors and corticosteroids.

In some embodiments, the one or more immunosuppressive agents comprise CNIs, mTOR inhibitors, and antiproliferative agents. In some embodiments, the one or more immunosuppressive agents comprise CNIs, mTOR inhibitors, and corticosteroids. In some embodiments, the one or more immunosuppressive agents comprise CNIs, antiproliferative agents, and corticosteroids. In some embodiments, the one or more immunosuppressive agents comprise mTOR inhibitors, antiproliferative agents, and corticosteroids.

In some embodiments, the administration of the alpha-biased IL-2 variants allows tapering or withdrawal of the standard of care. In some embodiments, the administration of the alpha-biased IL-2 variants allows tapering or withdrawal of the immunosuppressive agents.

In some embodiments, the alpha-biased IL-2 variant is administered prior to the kidney transplantation. “Prior to the kidney transplant” is used interchangeably with “pre-transplant” herein. In some embodiments, the alpha-biased IL-2 variant is administered subsequent to the kidney transplantation. “Subsequent to the kidney transplant” is used interchangeably with “post-transplant” herein. In some embodiments, the alpha-biased IL-2 variant is administered both prior to a kidney transplantation and subsequent to the kidney transplantation.

In one aspect, the present invention provides, among other things, a method of conditioning a kidney transplant recipient by administering to the recipient an alpha-biased IL-2 variant. In some embodiments, the alpha-biased IL-2 variant is administered prior to the kidney transplant at a therapeutically effective amount. In some embodiments, the alpha-biased IL-2 variant is further administered subsequent to the kidney transplantation. In some embodiments, the alpha-biased IL-2 variant is further administered simultaneously with the kidney transplantation.

### Combination Therapy

In some embodiments, the alpha-biased IL-2 variant described herein are used in combination with other therapies. In some embodiments, the combination therapy comprises the alpha-biased IL-2 variant co-formulated with one or more additional therapeutic agents. In some embodiments, the combination therapy comprises the alpha-biased IL-2 variant co-administered with one or more additional therapeutic agents. In some embodiments, the combination comprises the alpha-biased IL-2 variant administered in conjunction with one or more additional therapeutic agents.

In some embodiments, the combination therapy comprises the alpha-biased IL-2 variant and one therapeutic agent. In some embodiments, the combination therapy comprises the alpha-biased IL-2 variant and two therapeutic agents. In some embodiments, the combination therapy comprises the alpha-biased IL-2 variant and three therapeutic agents. In some embodiments, the combination therapy comprises the alpha-biased IL-2 variant and four therapeutic agents. In some embodiments, the combination therapy comprises the alpha-biased IL-2 variant and five or more therapeutic agents.

In some embodiments, the combination therapy comprises the alpha-biased IL-2 variant and an immunosuppressive regime. An immunosuppressive regime includes one or more immunosuppressive agents. In some embodiments, the combination therapy comprises the alpha-biased IL-2 variant and an immunosuppressive agent. In some embodiments, the immunosuppressive agent comprises cyclosporine, tacrolimus, mycophenolate mofetil, prednisone, azathioprine, sirolimus (also known as rapamycin), daclizumab, or basiliximab.

In some embodiments, the immunosuppressive agent comprises cyclosporine. In some embodiments, the immunosuppressive agent comprises tacrolimus. In some embodiments, the immunosuppressive agent comprises mycophenolate mofetil. In some embodiments, the immunosuppressive agent comprises prednisone. In some embodiments, the immunosuppressive agent comprises azathioprine. In some embodiments, the immunosuppressive agent comprises sirolimus (also known as rapamycin). In some embodiments, the immunosuppressive agent comprises daclizumab. In some embodiments, the immunosuppressive agent comprises basiliximab.

In some embodiments, the alpha-biased IL-2 variant is co-administered with the immunosuppressive agent. In some embodiments, the alpha-biased IL-2 variant is used in conjunction with the immunosuppressive agent. For example, and without limitation, the alpha-biased IL-2 variant and the immunosuppressive agent can be used in the same treatment plan. In some embodiments, the alpha-biased IL-2 variant is administered at a different frequency than the immunosuppressive agent. In some embodiments, the immunosuppressive agent is administered more frequently than the alpha-biased IL-2 variant.

In some embodiments, the immunosuppressive agent is administered prior to the kidney transplantation. In some embodiments, the immunosuppressive agent is administered subsequent to the kidney transplantation. In some embodiments, the immunosuppressive agent is co-administered with the kidney transplantation. In some embodiments, the immunosuppressive agent is first administered one day after the kidney transplantation.

In some embodiments, the immunosuppressive agent is administered daily. In some embodiments, the immunosuppressive agent is administered twice daily. In some embodiments, the immunosuppressive agent is administered in a therapeutically effective amount. In some embodiments, the immunosuppressive agent is administered such that the immunosuppressive agent remains in the patient at a therapeutically effective amount.

In some embodiments, the immunosuppressive agent is administered parenterally. In some embodiments, the immunosuppressive agent is administered intramuscularly. In some embodiments, the immunosuppressive agent is administered intravenously. In some embodiments, the immunosuppressive agent is administered subcutaneously.

In some embodiments, administration of the immunosuppressive agent is discontinued. In some embodiments, administration of the immunosuppressive agent is discontinued within 180 days of the kidney transplantation. In some embodiments, administration of the immunosuppressive agent is discontinued within 165 days of the kidney transplantation. In some embodiments, administration of the immunosuppressive agent is discontinued within 150 days of the kidney transplantation. In some embodiments, administration of the immunosuppressive agent is discontinued within 135 days of the kidney transplantation. In some embodiments, administration of the immunosuppressive agent is discontinued within 120 days of the kidney transplantation. In some embodiments, administration of the immunosuppressive agent is discontinued within 105 days of the kidney transplantation. In some embodiments, administration of the immunosuppressive agent is discontinued within 90 days of the kidney transplantation. In some embodiments, administration of the immunosuppressive agent is discontinued within 75 days of the kidney transplantation. In some embodiments, administration of the immunosuppressive agent is discontinued within 60 days of the kidney transplantation. In some embodiments, administration of the immunosuppressive agent is discontinued within 45 days of the kidney transplantation. In some embodiments, administration of the immunosuppressive agent is discontinued within 30 days of the kidney transplantation.

In some embodiments, administration of the immunosuppressive agent is discontinued within 180 days of the first administration of the alpha-biased IL-2 variant. In some embodiments, administration of the immunosuppressive agent is discontinued within 165 days of the first administration of the alpha-biased IL-2 variant. In some embodiments, administration of the immunosuppressive agent is discontinued within 150 days of the first administration of the alpha-biased IL-2 variant. In some embodiments, administration of the immunosuppressive agent is discontinued within 135 days of the first administration of the alpha-biased IL-2 variant. In some embodiments, administration of the immunosuppressive agent is discontinued within 120 days of the first administration of the alpha-biased IL-2 variant. In some embodiments, administration of the immunosuppressive agent is discontinued within 105 days of the first administration of the alpha-biased IL-2 variant. In some embodiments, administration of the immunosuppressive agent is discontinued within 90 days of the first administration of the alpha-biased IL-2 variant. In some embodiments, administration of the immunosuppressive agent is discontinued within 75 days of the first administration of the alpha-biased IL-2 variant. In some embodiments, administration of the immunosuppressive agent is discontinued within 60 days of the first administration of the alpha-biased IL-2 variant. In some embodiments, administration of the immunosuppressive agent is discontinued within 45 days of the first administration of the alpha-biased IL-2 variant. In some embodiments, administration of the immunosuppressive agent is discontinued within 30 days of the first administration of the alpha-biased IL-2 variant.

In some embodiments, the combination therapy comprises the alpha-biased IL-2 variant and a standard of care. In some embodiments, the alpha-biased IL-2 variant is co-administered with the standard of care. In some embodiments, the alpha-biased IL-2 variant is administered in conjunction with the standard of care.

In some embodiments, the standard of care comprises induction phase. In some embodiments, the standard of care comprises maintenance phase. In some embodiments, the standard of care comprises phase and maintenance phases.

In some embodiments, the standard of care comprises induction phase with rabbit antithymocyte globulin (rATG). In some embodiments, the standard of care comprises administration of one or more immunosuppressive agents. In some embodiments, the one or more immunosuppressive agents comprise calcineurin inhibitors (CNI), mammalian target of rapamycin (mTOR) inhibitors, antiproliferative agents, and/or corticosteroids.

In some embodiments, the standard of care comprises maintenance phase with an alpha-biased IL-2 variant.

In some embodiments, the standard of care comprises maintenance phase with a CNI. In some embodiments, the CNI is tacrolimus. In some embodiments, the CNI is cyclosporine A.

In some embodiments, the standard of care comprises maintenance phase with an mTOR inhibitor. In some embodiments, the mTOR inhibitor is rapamycin. In some embodiments, the mTOR inhibitor is everolimus.

In some embodiments, the standard of care comprises maintenance phase with an antiproliferative agent. In some embodiments, the antiproliferative agent is azathioprine. In some embodiments, the antiproliferative agent is mycophenolate mofetil (MMF).

In some embodiments, the standard of care comprises maintenance phase with a corticosteroid. In some embodiments, the corticosteroid is prednisone. In some embodiments, the corticosteroid is methylprednisolone. In some embodiments, the corticosteroid is dexamethasone. In some embodiments, the corticosteroid is hydrocortisone.

In some embodiments, the standard of care is discontinued. In some embodiments, the standard of care is discontinued within 180 days of the kidney transplantation. In some embodiments, the standard of care is discontinued within 165 days of the kidney transplantation. In some embodiments, the standard of care is discontinued within 150 days of the kidney transplantation. In some embodiments, the standard of care is discontinued within 135 days of the kidney transplantation. In some embodiments, the standard of care is discontinued within 120 days of the kidney transplantation. In some embodiments, the standard of care is discontinued within 105 days of the kidney transplantation. In some embodiments, the standard of care is discontinued within 90 days of the kidney transplantation. In some embodiments, the standard of care is discontinued within 75 days of the kidney transplantation. In some embodiments, the standard of care is discontinued within 60 days of the kidney transplantation. In some embodiments, the standard of care is discontinued within 45 days of the kidney transplantation. In some embodiments, the standard of care is discontinued within 30 days of the kidney transplantation.

In some embodiments, the standard of care is discontinued within 180 days of the first administration of the alpha-biased IL-2 variant. In some embodiments, the standard of care is discontinued within 165 days of the first administration of the alpha-biased IL-2 variant. In some embodiments, the standard of care is discontinued within 150 days of the first administration of the alpha-biased IL-2 variant. In some embodiments, the standard of care is discontinued within 135 days of the kidney transplantation. In some embodiments, the standard of care is discontinued within 120 days of the first administration of the alpha-biased IL-2 variant. In some embodiments, the standard of care is discontinued within 105 days of the first administration of the alpha-biased IL-2 variant. In some embodiments, the standard of care is discontinued within 90 days of the first administration of the alpha-biased IL-2 variant. In some embodiments, the standard of care is discontinued within 75 days of the first administration of the alpha-biased IL-2 variant. In some embodiments, the standard of care is discontinued within 60 days of the first administration of the alpha-biased IL-2 variant. In some embodiments, the standard of care is discontinued within 45 days of the first administration of the alpha-biased IL-2 variant. In some embodiments, the standard of care is discontinued within 30 days of the first administration of the alpha-biased IL-2 variant.

In some embodiments, the combination therapy comprises the alpha-biased IL-2 variant and a kidney transplant. In some embodiments, the combination therapy comprises the alpha-biased IL-2 agent, a kidney transplant, and one or more other therapeutic agents. In some embodiments, the combination therapy comprises the alpha-biased IL-2 agent, a kidney transplant, and a standard of care.

In some embodiments, the combination therapy comprises the alpha-biased IL-2 agent, a kidney transplant, and one or more immunosuppressive agents. In some embodiments, the combination therapy comprises the alpha-biased IL-2 agent, a kidney transplant, and an immunosuppressive regimen. In some embodiments, the combination therapy comprises the alpha-biased IL-2 agent, a kidney transplant, and an mTOR inhibitor. In some embodiments, the combination therapy comprises the alpha-biased IL-2 agent, a kidney transplant, and rapamycin.

In some embodiments, administration of the alpha-biased IL-2 variant results in a successful taper of the standard of care which lasts for at least two weeks. In some embodiments, administration of the alpha-biased IL-2 variant results in a successful taper of the standard of care which lasts for at least three weeks. In some embodiments, administration of the alpha-biased IL-2 variant results in a successful taper of the standard of care which lasts for at least four weeks. In some embodiments, administration of the alpha-biased IL-2 variant results in a successful taper of the standard of care which lasts for at least one month. In some embodiments, administration of the alpha-biased IL-2 variant results in a successful taper of the standard of care which lasts for at least two months. In some embodiments, administration of the alpha-biased IL-2 variant results in a successful taper of the standard of care which lasts for at least three months. In some embodiments, administration of the alpha-biased IL-2 variant results in a successful taper of the standard of care which lasts for at least four months. In some embodiments, administration of the alpha-biased IL-2 variant results in a successful taper of the standard of care which lasts for at least five months. In some embodiments, administration of the alpha-biased IL-2 variant results in a successful taper of the standard of care which lasts for at least six months. In some embodiments, administration of the alpha-biased IL-2 variant results in a successful taper of the standard of care which lasts for at least seven months. In some embodiments, administration of the alpha-biased IL-2 variant results in a successful taper of the standard of care which lasts for at least eight months. In some embodiments, administration of the alpha-biased IL-2 variant results in a successful taper of the standard of care which lasts for at least nine months. In some embodiments, administration of the alpha-biased IL-2 variant results in a successful taper of the standard of care which lasts for at least ten months. In some embodiments, administration of the alpha-biased IL-2 variant results in a successful taper of the standard of care which lasts for at least eleven months. In some embodiments, administration of the alpha-biased IL-2 variant results in a successful taper of the standard of care which lasts for at least twelve months. In some embodiments, administration of the alpha-biased IL-2 variant results in a successful taper of the standard of care which lasts for at least one year. In some embodiments, administration of the alpha-biased IL-2 variant results in a successful taper of the standard of care which lasts for at least two years. In some embodiments, administration of the alpha-biased IL-2 variant results in a successful taper of the standard of care which lasts for at least five years. In some embodiments, administration of the alpha-biased IL-2 variant results in a successful taper of the standard of care which lasts for the life of the recipient. In some embodiments, administration of the alpha-biased IL-2 variant results in a successful taper of the standard of care which lasts indefinitely.

In some embodiments, administration of the alpha-biased IL-2 variant results in a successful taper of the immunosuppressive agents which lasts for at least two weeks. In some embodiments, administration of the alpha-biased IL-2 variant results in a successful taper of the immunosuppressive agents which lasts for at least three weeks. In some embodiments, administration of the alpha-biased IL-2 variant results in a successful taper of the immunosuppressive agents which lasts for at least four weeks. In some embodiments, administration of the alpha-biased IL-2 variant results in a successful taper of the immunosuppressive agents which lasts for at least one month. In some embodiments, administration of the alpha-biased IL-2 variant results in a successful taper of the immunosuppressive agents which lasts for at least two months. In some embodiments, administration of the alpha-biased IL-2 variant results in a successful taper of the immunosuppressive agents which lasts for at least three months. In some embodiments, administration of the alpha-biased IL-2 variant results in a successful taper of the immunosuppressive agents which lasts for at least four months. In some embodiments, administration of the alpha-biased IL-2 variant results in a successful taper of the immunosuppressive agents which lasts for at least five months. In some embodiments, administration of the alpha-biased IL-2 variant results in a successful taper of the immunosuppressive agents which lasts for at least six months. In some embodiments, administration of the alpha-biased IL-2 variant results in a successful taper of the immunosuppressive agents which lasts for at least seven months. In some embodiments, administration of the alpha-biased IL-2 variant results in a successful taper of the immunosuppressive agents which lasts for at least eight months. In some embodiments, administration of the alpha-biased IL-2 variant results in a successful taper of the immunosuppressive agents which lasts for at least nine months. In some embodiments, administration of the alpha-biased IL-2 variant results in a successful taper of the immunosuppressive agents which lasts for at least ten months. In some embodiments, administration of the alpha-biased IL-2 variant results in a successful taper of the immunosuppressive agents which lasts for at least eleven months. In some embodiments, administration of the alpha-biased IL-2 variant results in a successful taper of the immunosuppressive agents which lasts for at least twelve months. In some embodiments, administration of the alpha-biased IL-2 variant results in a successful taper of the immunosuppressive agents which lasts for at least one year. In some embodiments, administration of the alpha-biased IL-2 variant results in a successful taper of the immunosuppressive agents which lasts for at least two years. In some embodiments, administration of the alpha-biased IL-2 variant results in a successful taper of the immunosuppressive agents which lasts for at least five years. In some embodiments, administration of the alpha-biased IL-2 variant results in a successful taper of the immunosuppressive agents which lasts for the life of the recipient. In some embodiments, administration of the alpha-biased IL-2 variant results in a successful taper of the immunosuppressive agents which lasts indefinitely.

In some embodiments, administration of the alpha-biased IL-2 variant results in a successful taper of the calcineurin inhibitors which lasts for at least two weeks. In some embodiments, administration of the alpha-biased IL-2 variant results in a successful taper of the calcineurin inhibitors which lasts for at least three weeks. In some embodiments, administration of the alpha-biased IL-2 variant results in a successful taper of the calcineurin inhibitors which lasts for at least four weeks. In some embodiments, administration of the alpha-biased IL-2 variant results in a successful taper of the calcineurin inhibitors which lasts for at least one month. In some embodiments, administration of the alpha-biased IL-2 variant results in a successful taper of the calcineurin inhibitors which lasts for at least two months. In some embodiments, administration of the alpha-biased IL-2 variant results in a successful taper of the calcineurin inhibitors which lasts for at least three months. In some embodiments, administration of the alpha-biased IL-2 variant results in a successful taper of the calcineurin inhibitors which lasts for at least four months. In some embodiments, administration of the alpha-biased IL-2 variant results in a successful taper of the calcineurin inhibitors which lasts for at least five months. In some embodiments, administration of the alpha-biased IL-2 variant results in a successful taper of the calcineurin inhibitors which lasts for at least six months. In some embodiments, administration of the alpha-biased IL-2 variant results in a successful taper of the calcineurin inhibitors which lasts for at least seven months. In some embodiments, administration of the alpha-biased IL-2 variant results in a successful taper of the calcineurin inhibitors which lasts for at least eight months. In some embodiments, administration of the alpha-biased IL-2 variant results in a successful taper of the calcineurin inhibitors which lasts for at least nine months. In some embodiments, administration of the alpha-biased IL-2 variant results in a successful taper of the calcineurin inhibitors which lasts for at least ten months. In some embodiments, administration of the alpha-biased IL-2 variant results in a successful taper of the calcineurin inhibitors which lasts for at least eleven months. In some embodiments, administration of the alpha-biased IL-2 variant results in a successful taper of the calcineurin inhibitors which lasts for at least twelve months. In some embodiments, administration of the alpha-biased IL-2 variant results in a successful taper of the calcineurin inhibitors which lasts for at least one year. In some embodiments, administration of the alpha-biased IL-2 variant results in a successful taper of the calcineurin inhibitors which lasts for at least two years. In some embodiments, administration of the alpha-biased IL-2 variant results in a successful taper of the calcineurin inhibitors which lasts for at least five years. In some embodiments, administration of the alpha-biased IL-2 variant results in a successful taper of the calcineurin inhibitors which lasts for the life of the recipient. In some embodiments, administration of the alpha-biased IL-2 variant results in a successful taper of the calcineurin inhibitors which lasts indefinitely.

In some embodiments, administration of the alpha-biased IL-2 variant results in a successful taper of the Tacrolimus which lasts for at least two weeks. In some embodiments, administration of the alpha-biased IL-2 variant results in a successful taper of the Tacrolimus which lasts for at least three weeks. In some embodiments, administration of the alpha-biased IL-2 variant results in a successful taper of the Tacrolimus which lasts for at least four weeks. In some embodiments, administration of the alpha-biased IL-2 variant results in a successful taper of the Tacrolimus which lasts for at least one month. In some embodiments, administration of the alpha-biased IL-2 variant results in a successful taper of the Tacrolimus which lasts for at least two months. In some embodiments, administration of the alpha-biased IL-2 variant results in a successful taper of the Tacrolimus which lasts for at least three months. In some embodiments, administration of the alpha-biased IL-2 variant results in a successful taper of the Tacrolimus which lasts for at least four months. In some embodiments, administration of the alpha-biased IL-2 variant results in a successful taper of the Tacrolimus which lasts for at least five months. In some embodiments, administration of the alpha-biased IL-2 variant results in a successful taper of the Tacrolimus which lasts for at least six months. In some embodiments, administration of the alpha-biased IL-2 variant results in a successful taper of the Tacrolimus which lasts for at least seven months. In some embodiments, administration of the alpha-biased IL-2 variant results in a successful taper of the Tacrolimus which lasts for at least eight months. In some embodiments, administration of the alpha-biased IL-2 variant results in a successful taper of the Tacrolimus which lasts for at least nine months. In some embodiments, administration of the alpha-biased IL-2 variant results in a successful taper of the Tacrolimus which lasts for at least ten months. In some embodiments, administration of the alpha-biased IL-2 variant results in a successful taper of the Tacrolimus which lasts for at least eleven months. In some embodiments, administration of the alpha-biased IL-2 variant results in a successful taper of the Tacrolimus which lasts for at least twelve months. In some embodiments, administration of the alpha-biased IL-2 variant results in a successful taper of the Tacrolimus which lasts for at least one year. In some embodiments, administration of the alpha-biased IL-2 variant results in a successful taper of the Tacrolimus which lasts for at least two years. In some embodiments, administration of the alpha-biased IL-2 variant results in a successful taper of the Tacrolimus which lasts for at least five years. In some embodiments, administration of the alpha-biased IL-2 variant results in a successful taper of the Tacrolimus which lasts for the life of the recipient. In some embodiments, administration of the alpha-biased IL-2 variant results in a successful taper of the Tacrolimus which lasts indefinitely.

In some embodiments, administration of the alpha-biased IL-2 variant results in a successful taper of the rapamycin which lasts for at least two weeks. In some embodiments, administration of the alpha-biased IL-2 variant results in a successful taper of the rapamycin which lasts for at least three weeks. In some embodiments, administration of the alpha-biased IL-2 variant results in a successful taper of the rapamycin which lasts for at least four weeks. In some embodiments, administration of the alpha-biased IL-2 variant results in a successful taper of the rapamycin which lasts for at least one month. In some embodiments, administration of the alpha-biased IL-2 variant results in a successful taper of the rapamycin which lasts for at least two months. In some embodiments, administration of the alpha-biased IL-2 variant results in a successful taper of the rapamycin which lasts for at least three months. In some embodiments, administration of the alpha-biased IL-2 variant results in a successful taper of the rapamycin which lasts for at least four months. In some embodiments, administration of the alpha-biased IL-2 variant results in a successful taper of the rapamycin which lasts for at least five months. In some embodiments, administration of the alpha-biased IL-2 variant results in a successful taper of the rapamycin which lasts for at least six months. In some embodiments, administration of the alpha-biased IL-2 variant results in a successful taper of the rapamycin which lasts for at least seven months. In some embodiments, administration of the alpha-biased IL-2 variant results in a successful taper of the rapamycin which lasts for at least eight months. In some embodiments, administration of the alpha-biased IL-2 variant results in a successful taper of the rapamycin which lasts for at least nine months. In some embodiments, administration of the alpha-biased IL-2 variant results in a successful taper of the rapamycin which lasts for at least ten months. In some embodiments, administration of the alpha-biased IL-2 variant results in a successful taper of the rapamycin which lasts for at least eleven months. In some embodiments, administration of the alpha-biased IL-2 variant results in a successful taper of the rapamycin which lasts for at least twelve months. In some embodiments, administration of the alpha-biased IL-2 variant results in a successful taper of the rapamycin which lasts for at least one year. In some embodiments, administration of the alpha-biased IL-2 variant results in a successful taper of the rapamycin which lasts for at least two years. In some embodiments, administration of the alpha-biased IL-2 variant results in a successful taper of the rapamycin which lasts for at least five years. In some embodiments, administration of the alpha-biased IL-2 variant results in a successful taper of the rapamycin which lasts for the life of the recipient. In some embodiments, administration of the alpha-biased IL-2 variant results in a successful taper of the rapamycin which lasts indefinitely.

In some embodiments, administration of the alpha-biased IL-2 variant results in a successful taper of the mycophenolate mofetil (MMF) which lasts for at least two weeks. In some embodiments, administration of the alpha-biased IL-2 variant results in a successful taper of the mycophenolate mofetil (MMF) which lasts for at least three weeks. In some embodiments, administration of the alpha-biased IL-2 variant results in a successful taper of the mycophenolate mofetil (MMF) which lasts for at least four weeks. In some embodiments, administration of the alpha-biased IL-2 variant results in a successful taper of the mycophenolate mofetil (MMF) which lasts for at least one month. In some embodiments, administration of the alpha-biased IL-2 variant results in a successful taper of the mycophenolate mofetil (MMF) which lasts for at least two months. In some embodiments, administration of the alpha-biased IL-2 variant results in a successful taper of the mycophenolate mofetil (MMF) which lasts for at least three months. In some embodiments, administration of the alpha-biased IL-2 variant results in a successful taper of the mycophenolate mofetil (MMF) which lasts for at least four months. In some embodiments, administration of the alpha-biased IL-2 variant results in a successful taper of the mycophenolate mofetil (MMF) which lasts for at least five months. In some embodiments, administration of the alpha-biased IL-2 variant results in a successful taper of the mycophenolate mofetil (MMF) which lasts for at least six months. In some embodiments, administration of the alpha-biased IL-2 variant results in a successful taper of the mycophenolate mofetil (MMF) which lasts for at least seven months. In some embodiments, administration of the alpha-biased IL-2 variant results in a successful taper of the mycophenolate mofetil (MMF) which lasts for at least eight months. In some embodiments, administration of the alpha-biased IL-2 variant results in a successful taper of the mycophenolate mofetil (MMF) which lasts for at least nine months. In some embodiments, administration of the alpha-biased IL-2 variant results in a successful taper of the mycophenolate mofetil (MMF) which lasts for at least ten months. In some embodiments, administration of the alpha-biased IL-2 variant results in a successful taper of the mycophenolate mofetil (MMF) which lasts for at least eleven months. In some embodiments, administration of the alpha-biased IL-2 variant results in a successful taper of the mycophenolate mofetil (MMF) which lasts for at least twelve months. In some embodiments, administration of the alpha-biased IL-2 variant results in a successful taper of the mycophenolate mofetil (MMF) which lasts for at least one year. In some embodiments, administration of the alpha-biased IL-2 variant results in a successful taper of the mycophenolate mofetil (MMF) which lasts for at least two years. In some embodiments, administration of the alpha-biased IL-2 variant results in a successful taper of the mycophenolate mofetil (MMF) which lasts for at least five years. In some embodiments, administration of the alpha-biased IL-2 variant results in a successful taper of the mycophenolate mofetil (MMF) which lasts for the life of the recipient. In some embodiments, administration of the alpha-biased IL-2 variant results in a successful taper of the mycophenolate mofetil (MMF) which lasts indefinitely.

In some embodiments, administration of the alpha-biased IL-2 variant results in a successful taper of the corticosteroids which lasts for at least two weeks. In some embodiments, administration of the alpha-biased IL-2 variant results in a successful taper of the corticosteroids which lasts for at least three weeks. In some embodiments, administration of the alpha-biased IL-2 variant results in a successful taper of the corticosteroids which lasts for at least four weeks. In some embodiments, administration of the alpha-biased IL-2 variant results in a successful taper of the corticosteroids which lasts for at least one month. In some embodiments, administration of the alpha-biased IL-2 variant results in a successful taper of the corticosteroids which lasts for at least two months. In some embodiments, administration of the alpha-biased IL-2 variant results in a successful taper of the corticosteroids which lasts for at least three months. In some embodiments, administration of the alpha-biased IL-2 variant results in a successful taper of the corticosteroids which lasts for at least four months. In some embodiments, administration of the alpha-biased IL-2 variant results in a successful taper of the corticosteroids which lasts for at least five months. In some embodiments, administration of the alpha-biased IL-2 variant results in a successful taper of the corticosteroids which lasts for at least six months. In some embodiments, administration of the alpha-biased IL-2 variant results in a successful taper of the corticosteroids which lasts for at least seven months. In some embodiments, administration of the alpha-biased IL-2 variant results in a successful taper of the corticosteroids which lasts for at least eight months. In some embodiments, administration of the alpha-biased IL-2 variant results in a successful taper of the corticosteroids which lasts for at least nine months. In some embodiments, administration of the alpha-biased IL-2 variant results in a successful taper of the corticosteroids which lasts for at least ten months. In some embodiments, administration of the alpha-biased IL-2 variant results in a successful taper of the corticosteroids which lasts for at least eleven months. In some embodiments, administration of the alpha-biased IL-2 variant results in a successful taper of the corticosteroids which lasts for at least twelve months. In some embodiments, administration of the alpha-biased IL-2 variant results in a successful taper of the corticosteroids which lasts for at least one year. In some embodiments, administration of the alpha-biased IL-2 variant results in a successful taper of the corticosteroids which lasts for at least two years. In some embodiments, administration of the alpha-biased IL-2 variant results in a successful taper of the corticosteroids which lasts for at least five years. In some embodiments, administration of the alpha-biased IL-2 variant results in a successful taper of the corticosteroids which lasts for the life of the recipient. In some embodiments, administration of the alpha-biased IL-2 variant results in a successful taper of the corticosteroids which lasts indefinitely.

In some embodiments, administration of the alpha-biased IL-2 variant results in withdrawal or weaning of the standard of care which lasts for at least two weeks. In some embodiments, administration of the alpha-biased IL-2 variant results in withdrawal or weaning of the standard of care which lasts for at least three weeks. In some embodiments, administration of the alpha-biased IL-2 variant results in withdrawal or weaning of the standard of care which lasts for at least four weeks. In some embodiments, administration of the alpha-biased IL-2 variant results in withdrawal or weaning of the standard of care which lasts for at least one month. In some embodiments, administration of the alpha-biased IL-2 variant results in withdrawal or weaning of the standard of care which lasts for at least two months. In some embodiments, administration of the alpha-biased IL-2 variant results in withdrawal or weaning of the standard of care which lasts for at least three months. In some embodiments, administration of the alpha-biased IL-2 variant results in withdrawal or weaning of the standard of care which lasts for at least four months. In some embodiments, administration of the alpha-biased IL-2 variant results in a withdrawal or weaning of the standard of care which lasts for at least five months. In some embodiments, administration of the alpha-biased IL-2 variant results in a withdrawal or weaning of the standard of care which lasts for at least six months. In some embodiments, administration of the alpha-biased IL-2 variant results in a withdrawal or weaning of the standard of care which lasts for at least seven months. In some embodiments, administration of the alpha-biased IL-2 variant results in a withdrawal or weaning of the standard of care which lasts for at least eight months. In some embodiments, administration of the alpha-biased IL-2 variant results in a withdrawal or weaning of the standard of care which lasts for at least nine months. In some embodiments, administration of the alpha-biased IL-2 variant results in a withdrawal or weaning of the standard of care which lasts for at least ten months. In some embodiments, administration of the alpha-biased IL-2 variant results in a withdrawal or weaning of the standard of care which lasts for at least eleven months. In some embodiments, administration of the alpha-biased IL-2 variant results in a withdrawal or weaning of the standard of care which lasts for at least twelve months. In some embodiments, administration of the alpha-biased IL-2 variant results in a withdrawal or weaning of the standard of care which lasts for at least one year. In some embodiments, administration of the alpha-biased IL-2 variant results in a withdrawal or weaning of the standard of care which lasts for at least two years. In some embodiments, administration of the alpha-biased IL-2 variant results in a withdrawal or weaning of the standard of care which lasts for at least five years. In some embodiments, administration of the alpha-biased IL-2 variant results in a withdrawal or weaning of the standard of care which lasts for the life of the recipient. In some embodiments, administration of the alpha-biased IL-2 variant results in a withdrawal or weaning of the standard of care which lasts indefinitely.

In some embodiments, administration of the alpha-biased IL-2 variant results in a withdrawal or weaning of the immunosuppressive agents which lasts for at least two weeks. In some embodiments, administration of the alpha-biased IL-2 variant results in a withdrawal or weaning of the immunosuppressive agents which lasts for at least three weeks. In some embodiments, administration of the alpha-biased IL-2 variant results in a withdrawal or weaning of the immunosuppressive agents which lasts for at least four weeks. In some embodiments, administration of the alpha-biased IL-2 variant results in a withdrawal or weaning of the immunosuppressive agents which lasts for at least one month. In some embodiments, administration of the alpha-biased IL-2 variant results in a withdrawal or weaning of the immunosuppressive agents which lasts for at least two months. In some embodiments, administration of the alpha-biased IL-2 variant results in a withdrawal or weaning of the immunosuppressive agents which lasts for at least three months. In some embodiments, administration of the alpha-biased IL-2 variant results in a withdrawal or weaning of the immunosuppressive agents which lasts for at least four months. In some embodiments, administration of the alpha-biased IL-2 variant results in a withdrawal or weaning of the immunosuppressive agents which lasts for at least five months. In some embodiments, administration of the alpha-biased IL-2 variant results in a withdrawal or weaning of the immunosuppressive agents which lasts for at least six months. In some embodiments, administration of the alpha-biased IL-2 variant results in a withdrawal or weaning of the immunosuppressive agents which lasts for at least seven months. In some embodiments, administration of the alpha-biased IL-2 variant results in a withdrawal or weaning of the immunosuppressive agents which lasts for at least eight months. In some embodiments, administration of the alpha-biased IL-2 variant results in a withdrawal or weaning of the immunosuppressive agents which lasts for at least nine months. In some embodiments, administration of the alpha-biased IL-2 variant results in a withdrawal or weaning of the immunosuppressive agents which lasts for at least ten months. In some embodiments, administration of the alpha-biased IL-2 variant results in a withdrawal or weaning of the immunosuppressive agents which lasts for at least eleven months. In some embodiments, administration of the alpha-biased IL-2 variant results in a withdrawal or weaning of the immunosuppressive agents which lasts for at least twelve months. In some embodiments, administration of the alpha-biased IL-2 variant results in a withdrawal or weaning of the immunosuppressive agents which lasts for at least one year. In some embodiments, administration of the alpha-biased IL-2 variant results in a withdrawal or weaning of the immunosuppressive agents which lasts for at least two years. In some embodiments, administration of the alpha-biased IL-2 variant results in a withdrawal or weaning of the immunosuppressive agents which lasts for at least five years. In some embodiments, administration of the alpha-biased IL-2 variant results in a withdrawal or weaning of the immunosuppressive agents which lasts for the life of the recipient. In some embodiments, administration of the alpha-biased IL-2 variant results in a withdrawal or weaning of the immunosuppressive agents which lasts indefinitely.

In some embodiments, administration of the alpha-biased IL-2 variant results in a withdrawal or weaning of the calcineurin inhibitors which lasts for at least two weeks. In some embodiments, administration of the alpha-biased IL-2 variant results in a withdrawal or weaning of the calcineurin inhibitors which lasts for at least three weeks. In some embodiments, administration of the alpha-biased IL-2 variant results in a withdrawal or weaning of the calcineurin inhibitors which lasts for at least four weeks. In some embodiments, administration of the alpha-biased IL-2 variant results in a withdrawal or weaning of the calcineurin inhibitors which lasts for at least one month. In some embodiments, administration of the alpha-biased IL-2 variant results in a withdrawal or weaning of the calcineurin inhibitors which lasts for at least two months. In some embodiments, administration of the alpha-biased IL-2 variant results in a withdrawal or weaning of the calcineurin inhibitors which lasts for at least three months. In some embodiments, administration of the alpha-biased IL-2 variant results in a withdrawal or weaning of the calcineurin inhibitors which lasts for at least four months. In some embodiments, administration of the alpha-biased IL-2 variant results in a withdrawal or weaning of the calcineurin inhibitors which lasts for at least five months. In some embodiments, administration of the alpha-biased IL-2 variant results in a withdrawal or weaning of the calcineurin inhibitors which lasts for at least six months. In some embodiments, administration of the alpha-biased IL-2 variant results in a withdrawal or weaning of the calcineurin inhibitors which lasts for at least seven months. In some embodiments, administration of the alpha-biased IL-2 variant results in a withdrawal or weaning of the calcineurin inhibitors which lasts for at least eight months. In some embodiments, administration of the alpha-biased IL-2 variant results in a withdrawal or weaning of the calcineurin inhibitors which lasts for at least nine months. In some embodiments, administration of the alpha-biased IL-2 variant results in a withdrawal or weaning of the calcineurin inhibitors which lasts for at least ten months. In some embodiments, administration of the alpha-biased IL-2 variant results in a withdrawal or weaning of the calcineurin inhibitors which lasts for at least eleven months. In some embodiments, administration of the alpha-biased IL-2 variant results in a withdrawal or weaning of the calcineurin inhibitors which lasts for at least twelve months. In some embodiments, administration of the alpha-biased IL-2 variant results in a withdrawal or weaning of the calcineurin inhibitors which lasts for at least one year. In some embodiments, administration of the alpha-biased IL-2 variant results in a withdrawal or weaning of the calcineurin inhibitors which lasts for at least two years. In some embodiments, administration of the alpha-biased IL-2 variant results in a withdrawal or weaning of the calcineurin inhibitors which lasts for at least five years. In some embodiments, administration of the alpha-biased IL-2 variant results in a withdrawal or weaning of the calcineurin inhibitors which lasts for the life of the recipient. In some embodiments, administration of the alpha-biased IL-2 variant results in a withdrawal or weaning of the calcineurin inhibitors which lasts indefinitely.

In some embodiments, administration of the alpha-biased IL-2 variant results in a withdrawal or weaning of the Tacrolimus which lasts for at least two weeks. In some embodiments, administration of the alpha-biased IL-2 variant results in a withdrawal or weaning of the Tacrolimus which lasts for at least three weeks. In some embodiments, administration of the alpha-biased IL-2 variant results in a withdrawal or weaning of the Tacrolimus which lasts for at least four weeks. In some embodiments, administration of the alpha-biased IL-2 variant results in a withdrawal or weaning of the Tacrolimus which lasts for at least one month. In some embodiments, administration of the alpha-biased IL-2 variant results in a withdrawal or weaning of the Tacrolimus which lasts for at least two months. In some embodiments, administration of the alpha-biased IL-2 variant results in a withdrawal or weaning of the Tacrolimus which lasts for at least three months. In some embodiments, administration of the alpha-biased IL-2 variant results in a withdrawal or weaning of the Tacrolimus which lasts for at least four months. In some embodiments, administration of the alpha-biased IL-2 variant results in a withdrawal or weaning of the Tacrolimus which lasts for at least five months. In some embodiments, administration of the alpha-biased IL-2 variant results in a withdrawal or weaning of the Tacrolimus which lasts for at least six months. In some embodiments, administration of the alpha-biased IL-2 variant results in a withdrawal or weaning of the Tacrolimus which lasts for at least seven months. In some embodiments, administration of the alpha-biased IL-2 variant results in a withdrawal or weaning of the Tacrolimus which lasts for at least eight months. In some embodiments, administration of the alpha-biased IL-2 variant results in a withdrawal or weaning of the Tacrolimus which lasts for at least nine months. In some embodiments, administration of the alpha-biased IL-2 variant results in a withdrawal or weaning of the Tacrolimus which lasts for at least ten months. In some embodiments, administration of the alpha-biased IL-2 variant results in a withdrawal or weaning of the Tacrolimus which lasts for at least eleven months. In some embodiments, administration of the alpha-biased IL-2 variant results in a withdrawal or weaning of the Tacrolimus which lasts for at least twelve months. In some embodiments, administration of the alpha-biased IL-2 variant results in a withdrawal or weaning of the Tacrolimus which lasts for at least one year. In some embodiments, administration of the alpha-biased IL-2 variant results in a withdrawal or weaning of the Tacrolimus which lasts for at least two years. In some embodiments, administration of the alpha-biased IL-2 variant results in a withdrawal or weaning of the Tacrolimus which lasts for at least five years. In some embodiments, administration of the alpha-biased IL-2 variant results in a withdrawal or weaning of the Tacrolimus which lasts for the life of the recipient. In some embodiments, administration of the alpha-biased IL-2 variant results in a withdrawal or weaning of the Tacrolimus which lasts indefinitely.

In some embodiments, administration of the alpha-biased IL-2 variant results in a withdrawal or weaning of the rapamycin which lasts for at least two weeks. In some embodiments, administration of the alpha-biased IL-2 variant results in a withdrawal or weaning of the rapamycin which lasts for at least three weeks. In some embodiments, administration of the alpha-biased IL-2 variant results in a withdrawal or weaning of the rapamycin which lasts for at least four weeks. In some embodiments, administration of the alpha-biased IL-2 variant results in a withdrawal or weaning of the rapamycin which lasts for at least one month. In some embodiments, administration of the alpha-biased IL-2 variant results in a withdrawal or weaning of the rapamycin which lasts for at least two months. In some embodiments, administration of the alpha-biased IL-2 variant results in a withdrawal or weaning of the rapamycin which lasts for at least three months. In some embodiments, administration of the alpha-biased IL-2 variant results in a withdrawal or weaning of the rapamycin which lasts for at least four months. In some embodiments, administration of the alpha-biased IL-2 variant results in a withdrawal or weaning of the rapamycin which lasts for at least five months. In some embodiments, administration of the alpha-biased IL-2 variant results in a withdrawal or weaning of the rapamycin which lasts for at least six months. In some embodiments, administration of the alpha-biased IL-2 variant results in a withdrawal or weaning of the rapamycin which lasts for at least seven months. In some embodiments, administration of the alpha-biased IL-2 variant results in a withdrawal or weaning of the rapamycin which lasts for at least eight months. In some embodiments, administration of the alpha-biased IL-2 variant results in a withdrawal or weaning of the rapamycin which lasts for at least nine months. In some embodiments, administration of the alpha-biased IL-2 variant results in a withdrawal or weaning of the rapamycin which lasts for at least ten months. In some embodiments, administration of the alpha-biased IL-2 variant results in a withdrawal or weaning of the rapamycin which lasts for at least eleven months. In some embodiments, administration of the alpha-biased IL-2 variant results in a withdrawal or weaning of the rapamycin which lasts for at least twelve months. In some embodiments, administration of the alpha-biased IL-2 variant results in a withdrawal or weaning of the rapamycin which lasts for at least one year. In some embodiments, administration of the alpha-biased IL-2 variant results in a withdrawal or weaning of the rapamycin which lasts for at least two years. In some embodiments, administration of the alpha-biased IL-2 variant results in a withdrawal or weaning of the rapamycin which lasts for at least five years. In some embodiments, administration of the alpha-biased IL-2 variant results in a withdrawal or weaning of the rapamycin which lasts for the life of the recipient. In some embodiments, administration of the alpha-biased IL-2 variant results in a withdrawal or weaning of the rapamycin which lasts indefinitely.

In some embodiments, administration of the alpha-biased IL-2 variant results in a withdrawal or weaning of the mycophenolate mofetil (MMF) which lasts for at least two weeks. In some embodiments, administration of the alpha-biased IL-2 variant results in a withdrawal or weaning of the mycophenolate mofetil (MMF) which lasts for at least three weeks. In some embodiments, administration of the alpha-biased IL-2 variant results in a withdrawal or weaning of the mycophenolate mofetil (MMF) which lasts for at least four weeks. In some embodiments, administration of the alpha-biased IL-2 variant results in a withdrawal or weaning of the mycophenolate mofetil (MMF) which lasts for at least one month. In some embodiments, administration of the alpha-biased IL-2 variant results in a withdrawal or weaning of the mycophenolate mofetil (MMF) which lasts for at least two months. In some embodiments, administration of the alpha-biased IL-2 variant results in a withdrawal or weaning of the mycophenolate mofetil (MMF) which lasts for at least three months. In some embodiments, administration of the alpha-biased IL-2 variant results in a withdrawal or weaning of the mycophenolate mofetil (MMF) which lasts for at least four months. In some embodiments, administration of the alpha-biased IL-2 variant results in a withdrawal or weaning of the mycophenolate mofetil (MMF) which lasts for at least five months. In some embodiments, administration of the alpha-biased IL-2 variant results in a withdrawal or weaning of the mycophenolate mofetil (MMF) which lasts for at least six months. In some embodiments, administration of the alpha-biased IL-2 variant results in a withdrawal or weaning of the mycophenolate mofetil (MMF) which lasts for at least seven months. In some embodiments, administration of the alpha-biased IL-2 variant results in a withdrawal or weaning of the mycophenolate mofetil (MMF) which lasts for at least eight months. In some embodiments, administration of the alpha-biased IL-2 variant results in a withdrawal or weaning of the mycophenolate mofetil (MMF) which lasts for at least nine months. In some embodiments, administration of the alpha-biased IL-2 variant results in a withdrawal or weaning of the mycophenolate mofetil (MMF) which lasts for at least ten months. In some embodiments, administration of the alpha-biased IL-2 variant results in a withdrawal or weaning of the mycophenolate mofetil (MMF) which lasts for at least eleven months. In some embodiments, administration of the alpha-biased IL-2 variant results in a withdrawal or weaning of the mycophenolate mofetil (MMF) which lasts for at least twelve months. In some embodiments, administration of the alpha-biased IL-2 variant results in a withdrawal or weaning of the mycophenolate mofetil (MMF) which lasts for at least one year. In some embodiments, administration of the alpha-biased IL-2 variant results in a withdrawal or weaning of the mycophenolate mofetil (MMF) which lasts for at least two years. In some embodiments, administration of the alpha-biased IL-2 variant results in a withdrawal or weaning of the mycophenolate mofetil (MMF) which lasts for at least five years. In some embodiments, administration of the alpha-biased IL-2 variant results in a withdrawal or weaning of the mycophenolate mofetil (MMF) which lasts for the life of the recipient. In some embodiments, administration of the alpha-biased IL-2 variant results in a withdrawal or weaning of the mycophenolate mofetil (MMF) which lasts indefinitely.

In some embodiments, administration of the alpha-biased IL-2 variant results in a withdrawal or weaning of the corticosteroids which lasts for at least two weeks. In some embodiments, administration of the alpha-biased IL-2 variant results in a withdrawal or weaning of the corticosteroids which lasts for at least three weeks. In some embodiments, administration of the alpha-biased IL-2 variant results in a withdrawal or weaning of the corticosteroids which lasts for at least four weeks. In some embodiments, administration of the alpha-biased IL-2 variant results in a withdrawal or weaning of the corticosteroids which lasts for at least one month. In some embodiments, administration of the alpha-biased IL-2 variant results in a withdrawal or weaning of the corticosteroids which lasts for at least two months. In some embodiments, administration of the alpha-biased IL-2 variant results in a withdrawal or weaning of the corticosteroids which lasts for at least three months. In some embodiments, administration of the alpha-biased IL-2 variant results in a withdrawal or weaning of the corticosteroids which lasts for at least four months. In some embodiments, administration of the alpha-biased IL-2 variant results in a withdrawal or weaning of the corticosteroids which lasts for at least five months. In some embodiments, administration of the alpha-biased IL-2 variant results in a withdrawal or weaning of the corticosteroids which lasts for at least six months. In some embodiments, administration of the alpha-biased IL-2 variant results in a withdrawal or weaning of the corticosteroids which lasts for at least seven months. In some embodiments, administration of the alpha-biased IL-2 variant results in a withdrawal or weaning of the corticosteroids which lasts for at least eight months. In some embodiments, administration of the alpha-biased IL-2 variant results in a withdrawal or weaning of the corticosteroids which lasts for at least nine months. In some embodiments, administration of the alpha-biased IL-2 variant results in a withdrawal or weaning of the corticosteroids which lasts for at least ten months. In some embodiments, administration of the alpha-biased IL-2 variant results in a withdrawal or weaning of the corticosteroids which lasts for at least eleven months. In some embodiments, administration of the alpha-biased IL-2 variant results in a withdrawal or weaning of the corticosteroids which lasts for at least twelve months. In some embodiments, administration of the alpha-biased IL-2 variant results in a withdrawal or weaning of the corticosteroids which lasts for at least one year. In some embodiments, administration of the alpha-biased IL-2 variant results in a withdrawal or weaning of the corticosteroids which lasts for at least two years. In some embodiments, administration of the alpha-biased IL-2 variant results in a withdrawal or weaning of the corticosteroids which lasts for at least five years. In some embodiments, administration of the alpha-biased IL-2 variant results in a withdrawal or weaning of the corticosteroids which lasts for the life of the recipient. In some embodiments, administration of the alpha-biased IL-2 variant results in a withdrawal or weaning of the corticosteroids which lasts indefinitely.

### Administration

### Therapeutic Outcomes

In some embodiments, administration of the alpha-biased IL-2 variant results in prolonged stability of the kidney transplant, survival of the recipient, and reduced rejection symptoms as compared to a control. In some embodiments, administration of the alpha-biased IL-2 variant results in prolonged stability of the kidney transplant, survival of the recipient, or reduced rejection symptoms as compared to a control.

In some embodiments, administration of the alpha-biased IL-2 variant results in prolonged stability of the kidney transplant. In some embodiments, administration of the alpha-biased IL-2 variant results in the survival of the recipient. In some embodiments, administration of the alpha-biased IL-2 variant results in reduced rejection symptoms as compared to a control. In some embodiments, administration of the alpha-biased IL-2 variant results in prolonged stability of the kidney transplant and survival of the recipient. In some embodiments, administration of the alpha-biased IL-2 variant results in prolonged stability of the kidney transplant and reduced rejection symptoms as compared to a control. In some embodiments, administration of the alpha-biased IL-2 variant results in survival of the recipient and reduced rejection symptoms as compared to a control.

In some embodiments, the control is historical data. In some embodiments, the control is a comparable recipient without the administration of the alpha-biased IL-2 variant. In some embodiments, the control is a comparable recipient receiving a standard of care. In some embodiments, the control is a comparable recipient receiving a standard of care without the administration of the alpha-biased IL-2 variant.

In some embodiments, administration of the alpha-biased IL-2 variant results in the kidney transplant being stable for at least 30 days. In some embodiments, administration of the alpha-biased IL-2 variant results in the kidney transplant being stable for at least 50 days. In some embodiments, administration of the alpha-biased IL-2 variant results in the kidney transplant being stable for at least 70 days. In some embodiments, administration of the alpha-biased IL-2 variant results in the kidney transplant being stable for at least 100 days. In some embodiments, administration of the alpha-biased IL-2 variant results in the kidney transplant being stable for at least 6 months. In some embodiments, administration of the alpha-biased IL-2 variant results in the kidney transplant being stable for at least 8 months. In some embodiments, administration of the alpha-biased IL-2 variant results in the kidney transplant being stable for at least 10 months. In some embodiments, administration of the alpha-biased IL-2 variant results in the kidney transplant being stable for at least 12 months. In some embodiments, administration of the alpha-biased IL-2 variant results in the kidney transplant being stable for at least 2 years. In some embodiments, administration of the alpha-biased IL-2 variant results in the kidney transplant being stable for at least 3 years. In some embodiments, administration of the alpha-biased IL-2 variant results in the kidney transplant being stable for at least 4 years. In some embodiments, administration of the alpha-biased IL-2 variant results in the kidney transplant being stable for at least 5 years. In some embodiments, administration of the alpha-biased IL-2 variant results in the kidney transplant being stable for at least 6 years. In some embodiments, administration of the alpha-biased IL-2 variant results in the kidney transplant being stable for at least 8 years. In some embodiments, administration of the alpha-biased IL-2 variant results in the kidney transplant being stable for at least 10 years. In some embodiments, the administration of the alpha-biased IL-2 variant results in the kidney transplant to be stable for the life of the recipient.

In some embodiments, administration of the alpha-biased IL-2 variant results in the kidney transplant being stable after administration of the alpha-biased IL-2 variant is discontinued. In some embodiments, administration of the alpha-biased IL-2 variant results in the kidney transplant being stable after administration of the standard of care is discontinued. In some embodiments, administration of the alpha-biased IL-2 variant results in the kidney transplant being stable after administration of the immunosuppressive agent is discontinued. In some embodiments, administration of the alpha-biased IL-2 variant results in the kidney transplant being stable after administration of the mTOR inhibitor is discontinued. In some embodiments, administration of the alpha-biased IL-2 variant results in the kidney transplant being stable after administration of the rapamycin is discontinued. In some embodiments, administration of the alpha-biased IL-2 variant results in the kidney transplant being stable after administration of the MMF is discontinued. In some embodiments, administration of the alpha-biased IL-2 variant results in the kidney transplant being stable after administration of the corticosteroid is discontinued. In some embodiments, administration of the alpha-biased IL-2 variant results in the kidney transplant being stable after administration of the Tacrolimus discontinued.

In some embodiments, administration of the alpha-biased IL-2 variant results in creatinine levels at or below a threshold value for at least one week, at least two weeks, at least three weeks, at least four weeks, at least five weeks, or at least six weeks. In some embodiments, administration of the alpha-biased IL-2 variant results in creatinine levels at or below a threshold value for at least one month, at least 2 months, at least 3 months, at least 4 months, at least 5 months, at least 6 months, at least 7 months, at least 8 months, at least 9 months, at least 10 months, at least 11 months, at least 12 months, at least 14 months, at least 16 months, at least 18 months, at least 20 months, at least 24 months, at least 30 months, at least 36 months, one year, two years, or five years. In some embodiments, administration of the alpha-biased IL-2 variant results in creatinine levels at or below a threshold value for the life of the recipient. In some embodiments, administration of the alpha-biased IL-2 variant results in creatinine levels at or below a threshold value indefinitely.

In some embodiments, administration of the alpha-biased IL-2 variant results in BUN levels at or below a threshold value for at least one week, at least two weeks, at least three weeks, at least four weeks, at least five weeks, or at least six weeks. In some embodiments, administration of the alpha-biased IL-2 variant results in BUN levels at or below a threshold value for at least one month, at least 2 months, at least 3 months, at least 4 months, at least 5 months, or at least 6 months, at least 7 months, at least 8 months, at least 9 months, at least 10 months, at least 11 months, at least 12 months, at least 14 months, at least 16 months, at least 18 months, at least 20 months, at least 24 months, at least 30 months, at least 36 months, one year, two years, or five years. In some embodiments, administration of the alpha-biased IL-2 variant results in BUN levels at or below a threshold value for the life of the recipient. In some embodiments, administration of the alpha-biased IL-2 variant results in BUN levels at or below a threshold value indefinitely.

In some embodiments, administration of the alpha-biased IL-2 variant results in an estimated GFR at or above a threshold value for at least one week, at least two weeks, at least three weeks, at least four weeks, at least five weeks, or at least six weeks. In some embodiments, administration of the alpha-biased IL-2 variant results in an estimated GFR at or above a threshold value for at least one month, at least 2 months, at least 3 months, at least 4 months, at least 5 months, or at least 6 months, at least 7 months, at least 8 months, at least 9 months, at least 10 months, at least 11 months, at least 12 months, at least 14 months, at least 16 months, at least 18 months, at least 20 months, at least 24 months, at least 30 months, at least 36 months, one year, two years, or five years. In some embodiments, administration of the alpha-biased IL-2 variant results in GFR levels at or below a threshold value for the life of the recipient. In some embodiments, administration of the alpha-biased IL-2 variant results in GFR levels at or below a threshold value indefinitely.

In some embodiments, administration of the alpha-biased IL-2 variant results in an estimated GFR (or percent of normal kidney function) at a threshold value of at least 50% (e.g., at least 60%, at least 70%, at least 75%, at least 80%, at least 85%, at least 90%, at least 95%). In some embodiments, administration of the alpha-biased IL-2 variant results in an estimated GFR at a threshold value of at least 60%. In some embodiments, administration of the alpha-biased IL-2 variant results in an estimated GFR at a threshold value of at least 70%. In some embodiments, administration of the alpha-biased IL-2 variant results in an estimated GFR at a threshold value of at least 75%. In some embodiments, administration of the alpha-biased IL-2 variant results in an estimated GFR at a threshold value of at least 80%. In some embodiments, administration of the alpha-biased IL-2 variant results in an estimated GFR at a threshold value of at least 85%. In some embodiments, administration of the alpha-biased IL-2 variant results in an estimated GFR at a threshold value of at least 90%. In some embodiments, administration of the alpha-biased IL-2 variant results in an estimated GFR at a threshold value of at least 95%. GFR can be estimated by any method well known in the art.

In some embodiments, administration of the alpha-biased IL-2 variant results in an increase in the percent of regulatory T cells (Tregs) in a population of T cells. In some embodiments, administration of the alpha-biased IL-2 variant results in an increase in the percent of CD4⁺CD25⁺FoxP3⁺ Treg cells in a population of CD4⁺ T cells.

In some embodiments, administration of the alpha-biased IL-2 variant does not substantially increase in the number of cytotoxic T lymphocytes (CTLs). In some embodiments, administration of the alpha-biased IL-2 variant does not substantially increase the number of NK cells.

### Dosing and Frequency

In some embodiments, the alpha-biased IL-2 variant is administered at a therapeutically effective amount. In some embodiments, the alpha-biased IL-2 variant is administered at a therapeutically effective amount and an administration interval.

In some embodiments, the alpha-biased IL-2 variant is administered prior to the kidney transplantation. “Prior to the kidney transplant” is also described as “pre-transplant” herein. In some embodiments, the alpha-biased IL-2 variant is administered subsequent to the kidney transplantation. “Subsequent to the kidney transplant” is also described as post-transplant herein. In some embodiments, the alpha-biased IL-2 variant is administered both prior to kidney transplantation and subsequent to kidney transplantation.

In some embodiments, the alpha-biased IL-2 variant is administered at least once, at least twice, at least three times, or at least four times prior to the kidney transplantation. In some embodiments, the alpha-biased IL-2 variant is administered at least once prior to the kidney transplantation. In some embodiments, the alpha-biased IL-2 variant is administered at least twice prior to the kidney transplantation. In some embodiments, the alpha-biased IL-2 variant is administered at least three times prior to the kidney transplantation. In some embodiments, the alpha-biased IL-2 variant is administered at least four times prior to the kidney transplantation.

In some embodiments, the alpha-biased IL-2 variant is administered 1 day prior to the kidney transplantation. In some embodiments, the alpha-biased IL-2 variant is administered 2 days prior to the kidney transplantation. In some embodiments, the alpha-biased IL-2 variant is administered 1 day prior to the kidney transplantation. In some embodiments, the alpha-biased IL-2 variant is administered 3 days prior to the kidney transplantation. In some embodiments, the alpha-biased IL-2 variant is administered 4 days prior to the kidney transplantation. In some embodiments, the alpha-biased IL-2 variant is administered 5 days prior to the kidney transplantation. In some embodiments, the alpha-biased IL-2 variant is administered 6 days prior to the kidney transplantation. In some embodiments, the alpha-biased IL-2 variant is administered 7 days prior to the kidney transplantation. In some embodiments, the alpha-biased IL-2 variant is administered 8 days prior to the kidney transplantation. In some embodiments, the alpha-biased IL-2 variant is administered 9 days prior to the kidney transplantation. In some embodiments, the alpha-biased IL-2 variant is administered 10 days prior to the kidney transplantation. In some embodiments, the alpha-biased IL-2 variant is administered 11 days prior to the kidney transplantation. In some embodiments, the alpha-biased IL-2 variant is administered 12 days prior to the kidney transplantation. In some embodiments, the alpha-biased IL-2 variant is administered 11 days prior to the kidney transplantation. In some embodiments, the alpha-biased IL-2 variant is administered 10 days prior to the kidney transplantation. In some embodiments, the alpha-biased IL-2 variant is administered 2 weeks prior to the kidney transplantation.

In some embodiments, the alpha-biased IL-2 variant is administered at least once, at least twice, at least three times, or at least four times subsequent to the kidney transplantation. In some embodiments, the alpha-biased IL-2 variant is administered at least once subsequent to the kidney transplantation. In some embodiments, the alpha-biased IL-2 variant is administered at least twice subsequent to the kidney transplantation. In some embodiments, the alpha-biased IL-2 variant is administered at least 3 times subsequent to the kidney transplantation. In some embodiments, the alpha-biased IL-2 variant is administered at least 4 times subsequent to the kidney transplantation. In some embodiments, the alpha-biased IL-2 variant is administered at least 5 times subsequent to the kidney transplantation. In some embodiments, the alpha-biased IL-2 variant is administered at least 6 times subsequent to the kidney transplantation. In some embodiments, the alpha-biased IL-2 variant is administered at least 7 times subsequent to the kidney transplantation. In some embodiments, the alpha-biased IL-2 variant is administered at least 8 times subsequent to the kidney transplantation. In some embodiments, the alpha-biased IL-2 variant is administered at least 9 times subsequent to the kidney transplantation. In some embodiments, the alpha-biased IL-2 variant is administered at least 10 times subsequent to the kidney transplantation. In some embodiments, the alpha-biased IL-2 variant is administered at least 15 times subsequent to the kidney transplantation. In some embodiments, the alpha-biased IL-2 variant is administered at least 20 times subsequent to the kidney transplantation. In some embodiments, the alpha-biased IL-2 variant is administered at least 30 times subsequent to the kidney transplantation. In some embodiments, the alpha-biased IL-2 variant is administered for the life of the recipient subsequent to the kidney transplantation.

In some embodiments, administration of the alpha-biased IL-2 variant is discontinued within 180 days of the kidney transplantation. In some embodiments, administration of the alpha-biased IL-2 variant is discontinued within 165 days of the kidney transplantation. In some embodiments, administration of the alpha-biased IL-2 variant is discontinued within 150 days of the kidney transplantation. In some embodiments, administration of the alpha-biased IL-2 variant is discontinued within 135 days of the kidney transplantation. In some embodiments, administration of the alpha-biased IL-2 variant is discontinued within 120 days of the kidney transplantation. In some embodiments, administration of the alpha-biased IL-2 variant is discontinued within 105 days of the kidney transplantation. In some embodiments, administration of the alpha-biased IL-2 variant is discontinued within 90 days of the kidney transplantation. In some embodiments, administration of the alpha-biased IL-2 variant is discontinued within 75 days of the kidney transplantation. In some embodiments, administration of the alpha-biased IL-2 variant is discontinued within 60 days of the kidney transplantation. In some embodiments, administration of the alpha-biased IL-2 variant is discontinued within 45 days of the kidney transplantation. In some embodiments, administration of the alpha-biased IL-2 variant is discontinued within 30 days of the kidney transplantation.

In some embodiments, the alpha-biased IL-2 variant is administered at a therapeutically effective amount and an administration interval. In some embodiments, the alpha-biased IL-2 variant is administered at an effective administration interval such that chronic dosing with the alpha-biased IL-2 variant may not be necessary.

In some embodiments, the alpha-biased IL-2 variant is administered at least once prior to the kidney transplantation and at least 2 times weekly subsequent to the kidney transplantation. In some embodiments, the alpha-biased IL-2 variant is administered at least once prior to the kidney transplantation and at least 3 times weekly subsequent to the kidney transplantation. In some embodiments, the alpha-biased IL-2 variant is administered at least once prior to the kidney transplantation and at least 5 times weekly subsequent to the kidney transplantation. In some embodiments, the alpha-biased IL-2 variant is administered at least once prior to the kidney transplantation and at least 8 times weekly subsequent to the kidney transplantation. In some embodiments, the alpha-biased IL-2 variant is administered at least once prior to the kidney transplantation and at least 10 times weekly subsequent to the kidney transplantation. In some embodiments, the alpha-biased IL-2 variant is administered at least once prior to the kidney transplantation and at least 12 times weekly subsequent to the kidney transplantation. In some embodiments, the alpha-biased IL-2 variant is administered at least once prior to the kidney transplantation and at least 15 times weekly subsequent to the kidney transplantation. In some embodiments, the alpha-biased IL-2 variant is administered at least once prior to the kidney transplantation and at least 18 times weekly subsequent to the kidney transplantation. In some embodiments, the alpha-biased IL-2 variant is administered at least once prior to the kidney transplantation and at least 20 times weekly subsequent to the kidney transplantation. In some embodiments, the alpha-biased IL-2 variant is administered at least once prior to the kidney transplantation and at least 25 times weekly subsequent to the kidney transplantation.

In some embodiments, the alpha-biased IL-2 variant is administered at least twice prior to the kidney transplantation and at least 2 times weekly subsequent to the kidney transplantation. In some embodiments, the alpha-biased IL-2 variant is administered at least twice prior to the kidney transplantation and at least 3 times weekly subsequent to the kidney transplantation. In some embodiments, the alpha-biased IL-2 variant is administered at least twice prior to the kidney transplantation and at least 5 times weekly subsequent to the kidney transplantation. In some embodiments, the alpha-biased IL-2 variant is administered at least twice prior to the kidney transplantation and at least 8 times weekly subsequent to the kidney transplantation. In some embodiments, the alpha-biased IL-2 variant is administered at least twice prior to the kidney transplantation and at least 10 times weekly subsequent to the kidney transplantation. In some embodiments, the alpha-biased IL-2 variant is administered at least twice prior to the kidney transplantation and at least 12 times weekly subsequent to the kidney transplantation. In some embodiments, the alpha-biased IL-2 variant is administered at least twice prior to the kidney transplantation and at least 15 times weekly subsequent to the kidney transplantation. In some embodiments, the alpha-biased IL-2 variant is administered at least twice prior to the kidney transplantation and at least 18 times weekly subsequent to the kidney transplantation. In some embodiments, the alpha-biased IL-2 variant is administered at least twice prior to the kidney transplantation and at least 20 times weekly subsequent to the kidney transplantation. In some embodiments, the alpha-biased IL-2 variant is administered at least twice prior to the kidney transplantation and at least 25 times weekly subsequent to the kidney transplantation.

In some embodiments, the alpha-biased IL-2 variant is administered at least three times prior to the kidney transplantation and at least 2 times weekly subsequent to the kidney transplantation. In some embodiments, the alpha-biased IL-2 variant is administered at least three times prior to the kidney transplantation and at least 3 times weekly subsequent to the kidney transplantation. In some embodiments, the alpha-biased IL-2 variant is administered at least three times prior to the kidney transplantation and at least 5 times weekly subsequent to the kidney transplantation. In some embodiments, the alpha-biased IL-2 variant is administered at least three times prior to the kidney transplantation and at least 8 times weekly subsequent to the kidney transplantation. In some embodiments, the alpha-biased IL-2 variant is administered at least three times prior to the kidney transplantation and at least 10 times weekly subsequent to the kidney transplantation. In some embodiments, the alpha-biased IL-2 variant is administered at least three times prior to the kidney transplantation and at least 12 times weekly subsequent to the kidney transplantation. In some embodiments, the alpha-biased IL-2 variant is administered at least three times prior to the kidney transplantation and at least 15 times weekly subsequent to the kidney transplantation. In some embodiments, the alpha-biased IL-2 variant is administered at least three times prior to the kidney transplantation and at least 18 times weekly subsequent to the kidney transplantation. In some embodiments, the alpha-biased IL-2 variant is administered at least three times prior to the kidney transplantation and at least 20 times weekly subsequent to the kidney transplantation. In some embodiments, the alpha-biased IL-2 variant is administered at least three times prior to the kidney transplantation and at least 25 times weekly subsequent to the kidney transplantation.

In some embodiments, the alpha-biased IL-2 variant is administered at a frequency that maintains a therapeutically effective level of the alpha-biased IL-2 variant in the patient's systems. In some embodiments, the alpha-biased IL-2 variant is administered at a frequency that maintains one or more beneficial therapeutic outcomes for the kidney transplant.

In some embodiments, the alpha-biased IL-2 variant is administered every 1, 2, 3, 4, 5, 6, or 7 days. In some embodiments, the alpha-biased IL-2 variant is administered every day, or every 1 day. In some embodiments, the alpha-biased IL-2 variant is administered every other day, or every 2 days. In some embodiments, the alpha-biased IL-2 variant is administered every 3 days. In some embodiments, the alpha-biased IL-2 variant is administered every 4 days. In some embodiments, the alpha-biased IL-2 variant is administered every 5 days. In some embodiments, the alpha-biased IL-2 variant is administered every 6 days. In some embodiments, the alpha-biased IL-2 agent is administered once every week, or every 7 days.

In some embodiments, the alpha-biased IL-2 variant is administered every 1, 2, 3, 4, 5, or 6 weeks. In some embodiments, the alpha-biased IL-2 variant is administered every week, or every 1 week. In some embodiments, the alpha-biased IL-2 variant is administered every 2 weeks. In some embodiments, the alpha-biased IL-2 variant is administered every 3 weeks. In some embodiments, the alpha-biased IL-2 variant is administered every 4 weeks. In some embodiments, the alpha-biased IL-2 variant is administered every 5 weeks. In some embodiments, the alpha-biased IL-2 variant is administered every 6. weeks.

In some embodiments, the alpha-biased IL-2 variant is administered every 1, 2, 3, 4, 5, or 6 months. In some embodiments, the alpha-biased IL-2 variant is administered once a month, or every 1 month. In some embodiments, the alpha-biased IL-2 variant is administered every 2 months. In some embodiments, the alpha-biased IL-2 variant is administered every 3 months. In some embodiments, the alpha-biased IL-2 variant is administered every 4 months. In some embodiments, the alpha-biased IL-2 variant is administered every 5 months. In some embodiments, the alpha-biased IL-2 variant is administered every 6 months.

In some embodiments, the alpha-biased IL-2 variant is administered at least once prior to the kidney transplantation and at least 2 times weekly subsequent to the kidney transplantation. In some embodiments, the alpha-biased IL-2 variant is administered at least once prior to the kidney transplantation and at least 3 times weekly subsequent to the kidney transplantation. In some embodiments, the alpha-biased IL-2 variant is administered at least once prior to the kidney transplantation and at least 5 times weekly subsequent to the kidney transplantation. In some embodiments, the alpha-biased IL-2 variant is administered at least once prior to the kidney transplantation and at least 8 times weekly subsequent to the kidney transplantation. In some embodiments, the alpha-biased IL-2 variant is administered at least once prior to the kidney transplantation and at least 10 times weekly subsequent to the kidney transplantation. In some embodiments, the alpha-biased IL-2 variant is administered at least once prior to the kidney transplantation and at least 12 times weekly subsequent to the kidney transplantation. In some embodiments, the alpha-biased IL-2 variant is administered at least once prior to the kidney transplantation and at least 15 times weekly subsequent to the kidney transplantation. In some embodiments, the alpha-biased IL-2 variant is administered at least once prior to the kidney transplantation and at least 18 times weekly subsequent to the kidney transplantation. In some embodiments, the alpha-biased IL-2 variant is administered at least once prior to the kidney transplantation and at least 20 times weekly subsequent to the kidney transplantation. In some embodiments, the alpha-biased IL-2 variant is administered at least once prior to the kidney transplantation and at least 25 times weekly subsequent to the kidney transplantation.

In some embodiments, the alpha-biased IL-2 variant is administered parenterally. Parenteral administration includes, for example and without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intratarsal injection and infusion. In some embodiments, the alpha-biased IL-2 variant is administered intramuscularly. In some embodiments, the alpha-biased IL-2 variant is administered intravenously. In some embodiments, the alpha-biased IL-2 variant is administered subcutaneously.

### Compositions

The alpha-biased IL-2 variants, and alpha-biased IL-2 fusion proteins, of the present invention (also referred to herein as “active compounds”) can be incorporated into pharmaceutical compositions suitable for administration. Such compositions typically comprise the alpha-biased IL-2 variants, or alpha-biased IL-2 fusion proteins, and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention is dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### EXAMPLES

While certain compounds, compositions, and methods of the present disclosure have been described with specificity in accordance with certain embodiments, the following examples serve only to illustrate the disclosure and are not intended to limit the same.

### Example 1—Alpha-Biased IL-2 Variants Preferentially Induce Regulatory T Cells

This example measures the effect of exemplary alpha-biased IL-2 variants in selectively expanding regulatory T cells (Tregs).

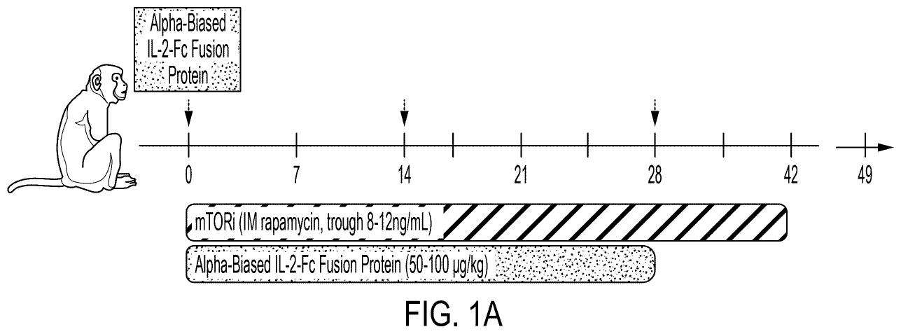

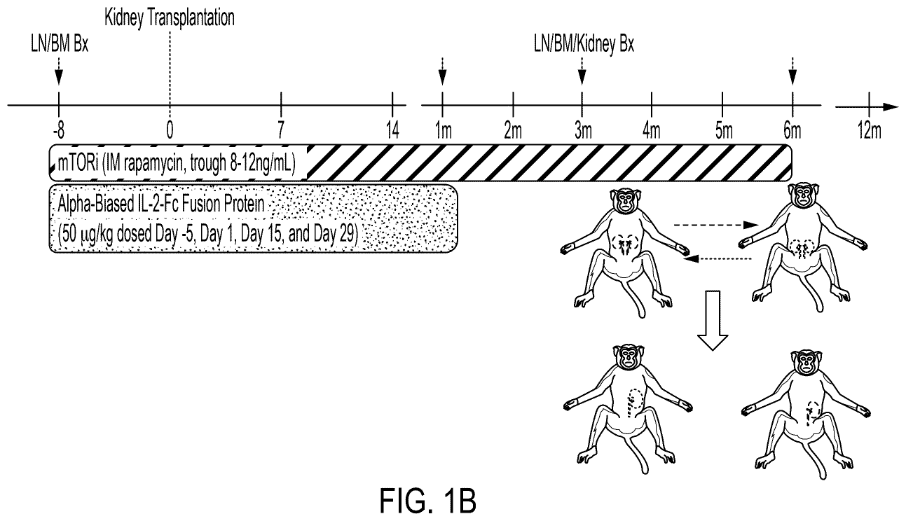

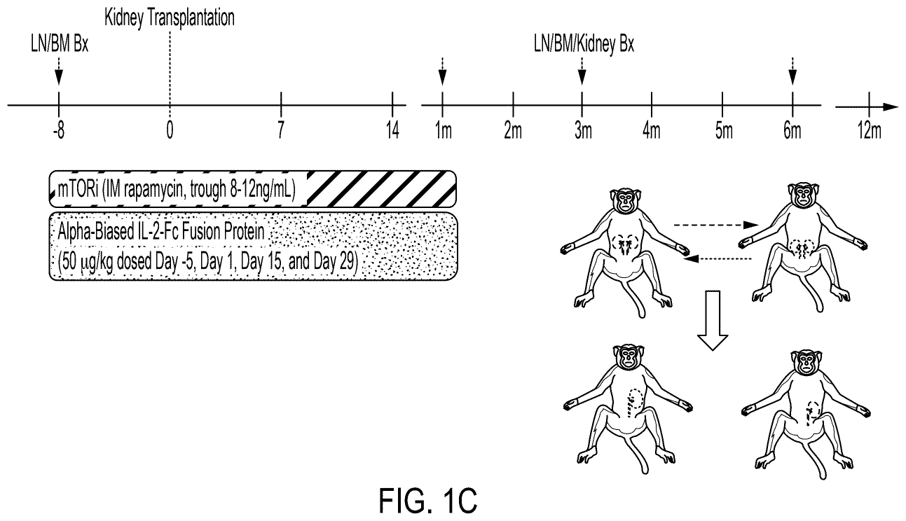

An exemplary alpha-biased IL-2 variant fused to an Fc domain of human IgG1 was prepared with a full length sequence according to SEQ ID NO: 27. The immunomodulatory effect of the exemplary alpha-biased IL-2-Fc fusion protein was assessed in naïve Rhesus monkeys by a study outlined in FIG. 1A. The alpha-biased IL-2-Fc fusion protein was administered subcutaneously on Days 0, 14, and 28 at a dose of 50 μg/kg or 100 μg/kg. Rapamycin was also administered daily from Day 0 by intramuscular injections, targeting a trough level of approximately 8-12 ng/mL. Treg populations (CD4⁺CD25⁺FoxP3⁺ and CD4⁺CD25⁺CD127^{lo}) and other immune cell populations including CD3⁺/CD8⁺ cytotoxic T cells (CTL) were analyzed using flow cytometry.

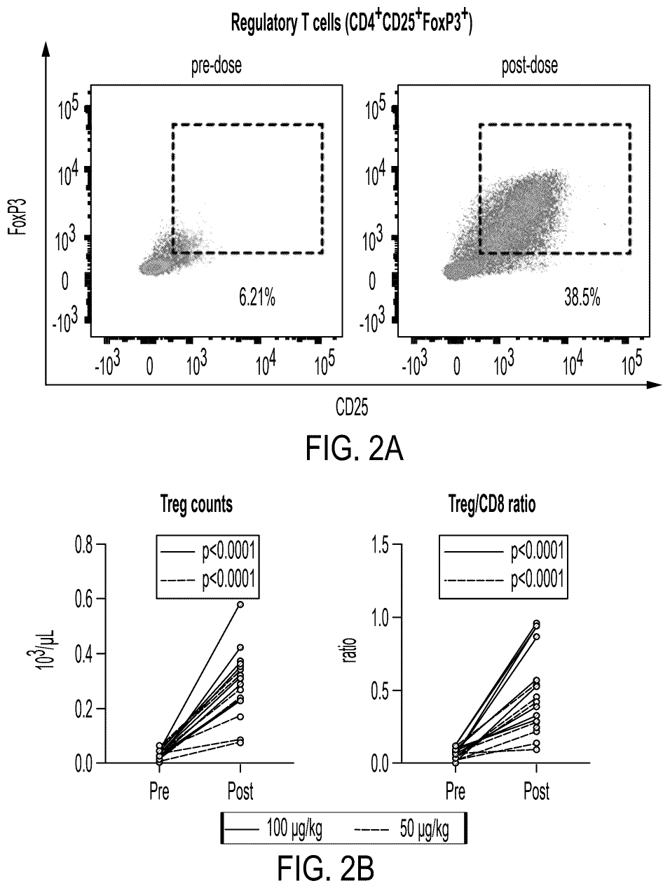

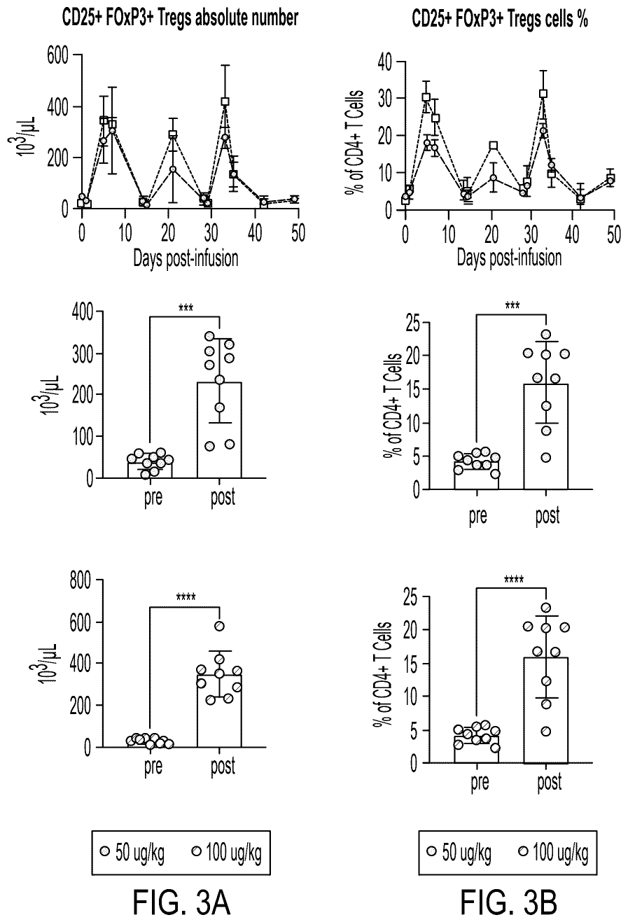

The absolute number and percent of Treg cells significantly increased over the course of treatment. As shown in FIG. 2A, the percent of Tregs increased to 38.5% from 6.21% as soon as 5-7 days after administration of the alpha-biased IL-2-Fc fusion protein. The absolute number of Tregs, as well as the ratio of Tregs:CD8 cells, significantly increased in a dose-dependent manner over the course of treatment as shown in FIG. 2B. This shows that the increase in Tregs did not coincide with an increase in cytotoxic T cells (CTLs). As shown in further in FIGS. 3A and 3B, both the number and percent of Tregs increased dose-dependently without signs of tachyphylaxis.

This example demonstrated that an exemplary alpha-biased IL-2 variant can selectively, and dose-dependently, expand Tregs in nonhuman primates.

### Example 2—Alpha-Biased IL-2 Variants Improve Kidney Transplant Outcomes in Nonhuman Primates

This example shows that administration of an exemplary alpha-biased IL-2 variant can improve long-term outcomes for kidney transplant recipients.

The effect of an exemplary alpha-biased IL-2-Fc fusion protein (SEQ ID NO: 27) on prolonged kidney allograft survival following transplant was studied in naïve Rhesus monkeys, as outlined in FIG. 1B. For the kidney transplant, animals were paired with ABO-compatible donors and mismatched for MHC haplotype of class I loci while allowing 1 or 2 MHC II haplotype matching. This degree of MHC mismatch is known to elicit a robust graft rejection and sensitization.

The alpha-biased IL-2-Fc fusion protein was administered subcutaneously at a dose of 50 μg/kg both pre- and post-transplant on Days −6, 1, 15, and 29. The kidney transplant was performed on Day 0, and rapamycin was administered daily by intramuscular injection, targeting a trough concentration of approximately 8-12 ng/mL. Rapamycin administration was discontinued on Day 180 post-transplant. As a control, five monkeys were only administered the daily rapamycin. Kidney allograft biopsies were performed at 1-month and 3-months post-transplant. Blood samples were collected regularly, and samples were either analyzed at ambient temperature or frozen and stored. The testing groups are summarized in Table A.

TABLE A
Testing groups for Example 2.

| Sample | Test Article |
|---|---|
| Sample B | Alpha-biased IL-2-Fc |
| Sample S | fusion protein and |
| Sample Y | Daily Rapamycin |
| Sample R | |
| Sample P | Daily Rapamycin |
| Sample M | |
| Sample N | |
| Sample E | |

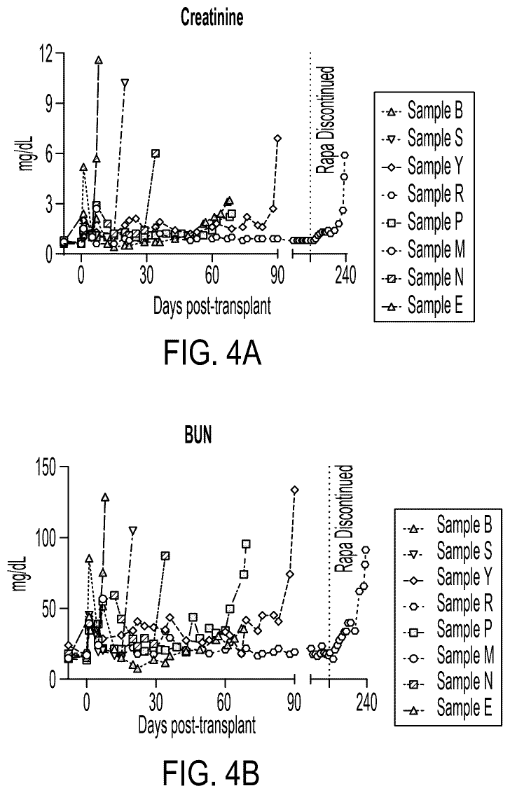

Creatinine and blood urea nitrogen (BUN) were stable in most cases for over 50 days, and in some cases over 235 days, post-transplant as shown in FIGS. 4A and 4B, respectively.

Several blood chemistry parameters were also tracked throughout the study. Levels of alanine aminotransferase (ALT), alkaline phosphatase (ALP), gamma-glutamyl transpeptidase (GGT), bilirubin, protein, albumin, globulin, cholesterol, phosphate, calcium, glucose, sodium, potassium, and chloride fell within the expected range for each parameter.

Blood samples were also analyzed for hematology parameters. Hematology parameters such has hemoglobin concentration, hematocrit levels, and cell counts for red blood cells, platelets, white blood cells, neutrophils, lymphocytes, monocytes, eosinophils, basophil, and neutrophils normally fell within their expected ranges.

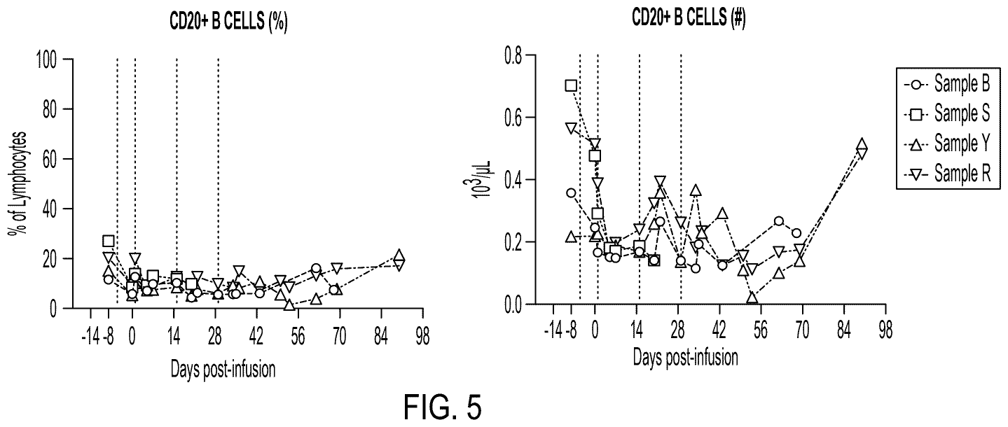

Different cell populations were also tracked throughout the study. B cells are a major contributor to acute and chronic antibody-mediated rejection in organ transplant. As CD20 is a general B cell marker expressed by a majority of B cells, the decrease in CD20⁺ B cells shown in FIG. 5 support a lack of transplant rejection.

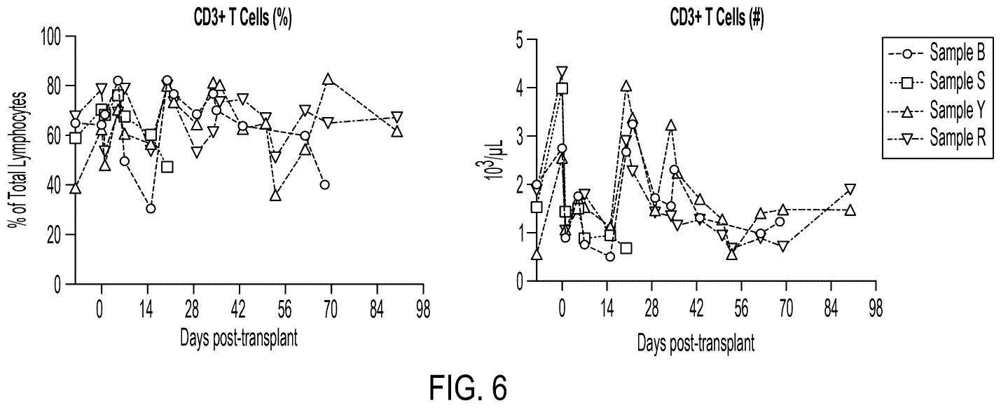

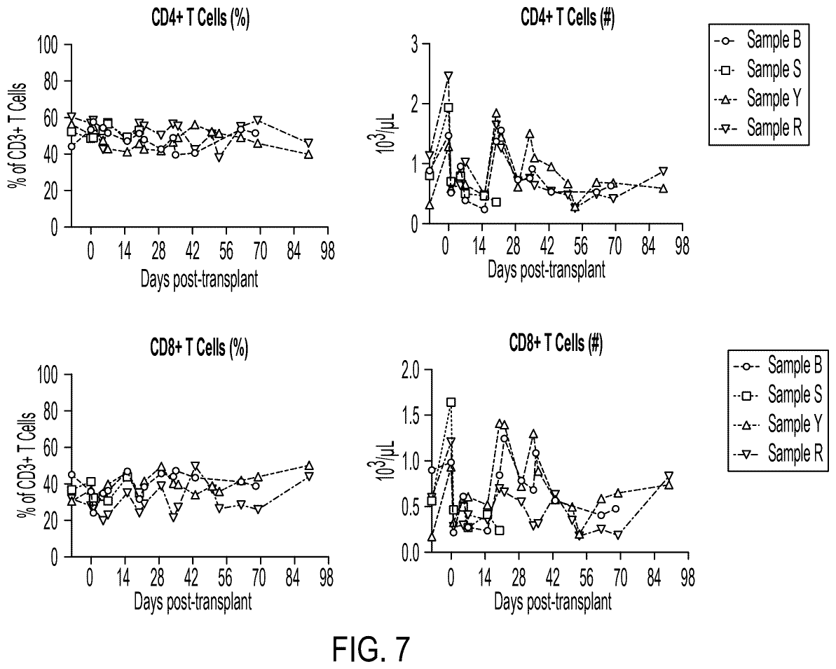

FIG. 6 shows the percent and absolute values for CD3⁺ T cells. FIG. 7 shows the percent of CD4⁺ and CD8⁺ T cells out of the total population of CD3⁺ T cells as well as the absolute number of CD4⁺ and CD8⁺ T cells in PBMCs.

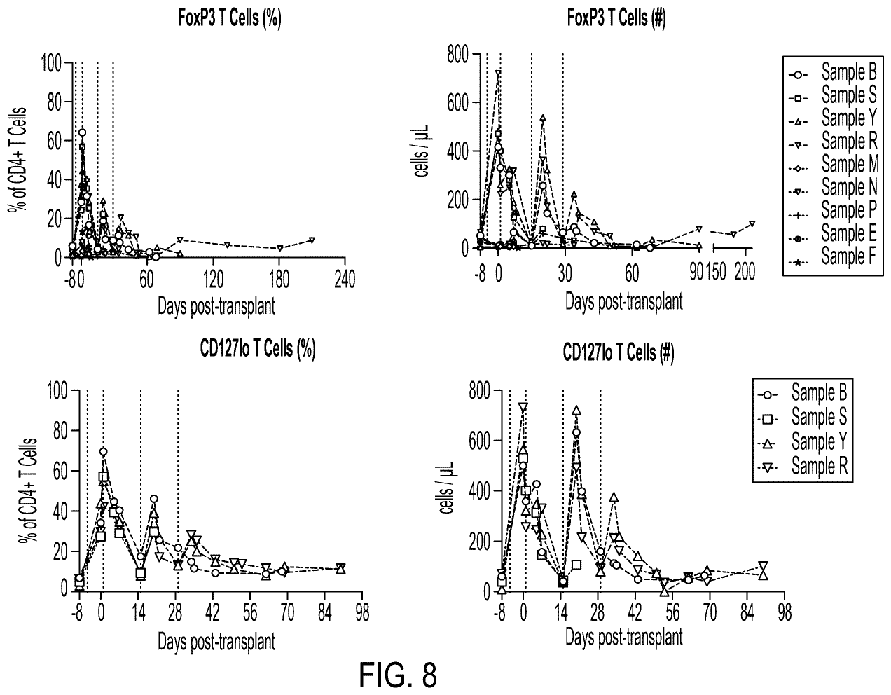

FIG. 8 shows the percents and absolute values for FoxP3⁺ CD4⁺ T cells and CD127^{lo} CD4⁺ Tregs in PBMCs. As shown, Tregs increase in both percent and number after administration of the alpha-biased IL-2-Fc fusion protein. A lack of Treg response, or Treg levels below baseline, would indicate transplant rejection.

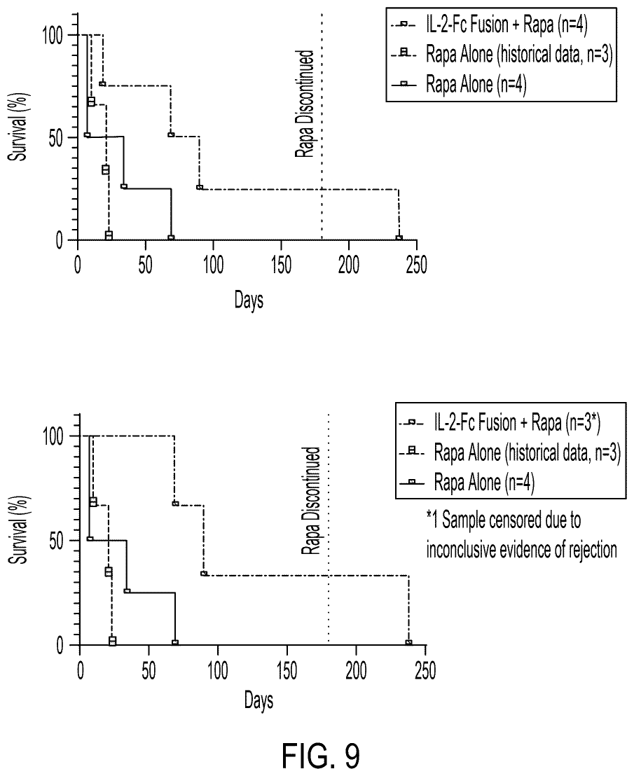

The survival for each testing group is summarized in FIG. 9 alongside historical data for rapamycin alone and control data for rapamycin alone. One monkey was terminated early in the alpha-biased IL-2-Fc fusion protein testing group due to excessive body weight loss. However, the histopathology did not indicate kidney rejection. Since it was suspected that the monkey was sick for another reason, the monkey was censored from the bottom graph demonstrating survival in FIG. 9.

Overall, prolonged kidney graft survival (over 50 days) was demonstrated in at least three of four Rhesus monkeys, with one Rhesus monkey surviving 235 days post-transplant. Further analysis will include single cell sequencing of the kidney biopsies and peripheral blood mononuclear cells (PBMCs) from the frozen blood samples.

### Example 3—Alpha-Biased IL-2 Variants Improve Kidney Transplant Outcomes in Nonhuman Primates after Discontinuation of Treatment

This example shows that administration of an exemplary alpha-biased IL-2 variant can improve long-term outcomes for kidney transplant recipients even after discontinuation of the alpha-biased IL-2 variant and rapamycin treatments.

The effect of an exemplary alpha-biased IL-2-Fc fusion protein (SEQ ID NO: 27) on prolonged kidney allograft survival following transplant and discontinuation of treatment was studied in naïve Rhesus monkeys, using similar methodology as Example 2, as shown in FIG. 1C. The alpha-biased IL-2-Fc fusion protein was administered subcutaneously at a dose of 50 μg/kg both pre- and post-transplant on Days −6, 1, 15, and 29. The kidney transplant was performed on Day 0, and rapamycin was administered daily by intramuscular injection, targeting a trough concentration of approximately 8-12 ng/mL. However, rapamycin administration was discontinued on Day 30 post-transplant. A control group was only administered the daily rapamycin. The testing groups are summarized in Table B.

TABLE B
Testing groups for Example 3.

| Sample | Test Article |
|---|---|
| Sample A | Alpha-biased IL-2-Fc |
| Sample J | fusion protein and |
| Sample I | Daily Rapamycin |
| Sample L | |
| Sample F | Daily Rapamycin |
| Sample H | |

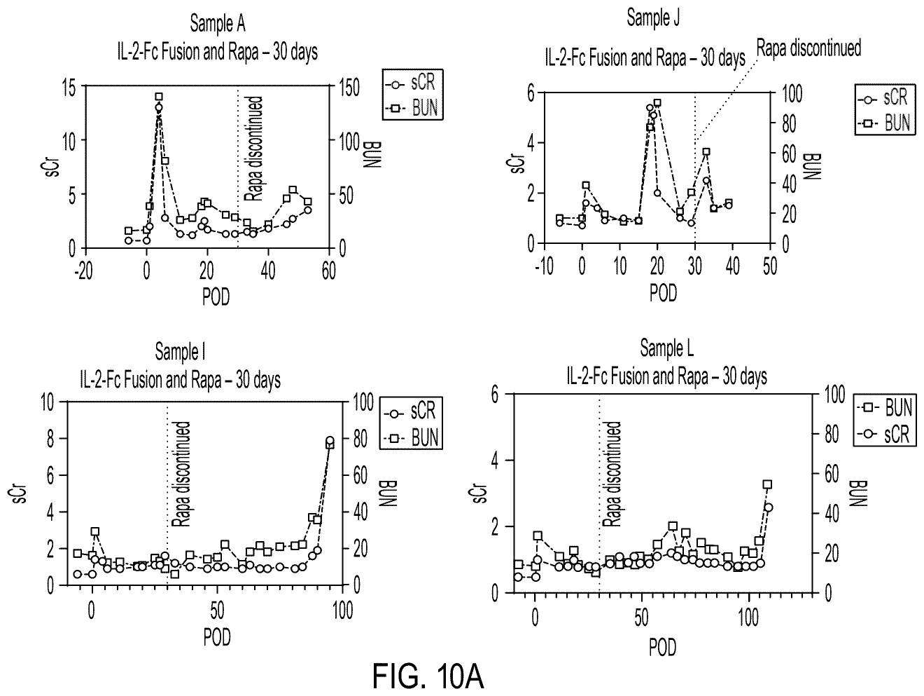

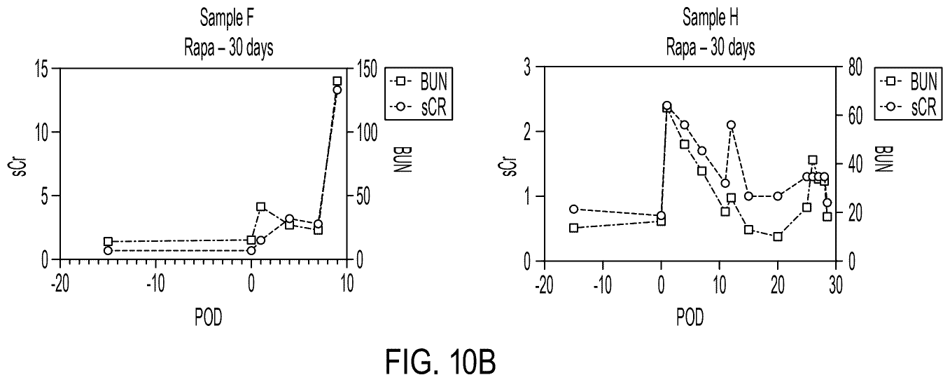

Serum creatinine (sCR) and blood urea nitrogen (BUN) levels were tracked throughout the study as shown in FIGS. 10A and 10B for the alpha-biased IL-2-Fc fusion protein testing group and control testing group, respectively. Monkeys in the control group were terminated less than 30 days post-transplant due to increases in sCR and BUN or excessive weight loss. In this example, rapamycin was discontinued at 30 days if the animals survived to day 30. Monkeys in the alpha-biased IL-2-Fc fusion protein testing group showed long-term stability of the kidney allograft, with sCR and BUN levels remaining stable for at least 40 days post-transplant in most cases. In some cases, the sCR and BUN levels were stable for around or over 110 days post-transplant and up to 82 days following complete withdrawal of immunosuppressive drugs.

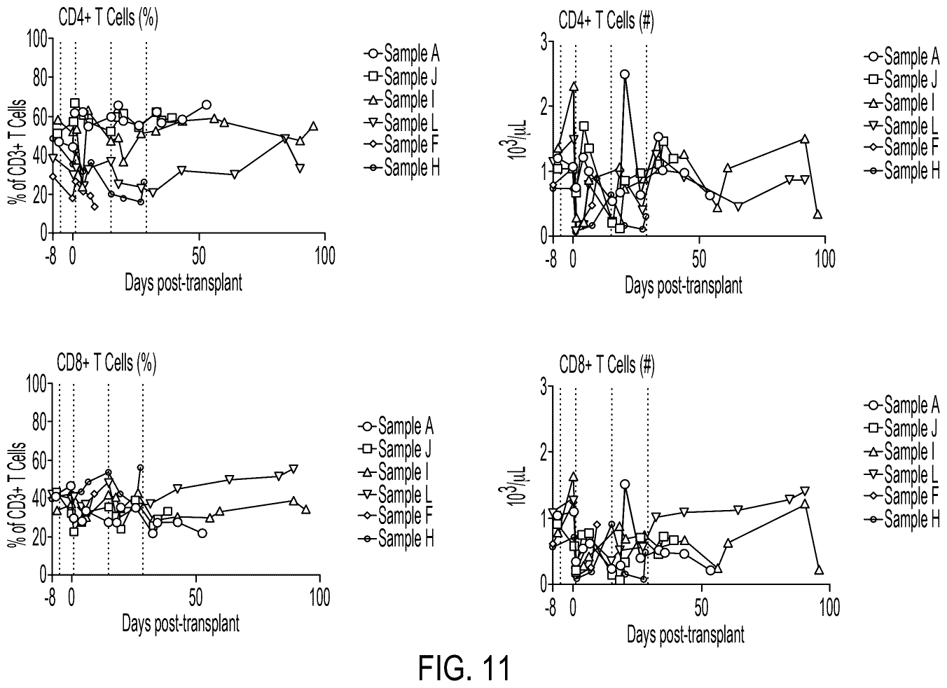

FIG. 11 shows the percent of CD4⁺ and CD8⁺ T cells out of the total population of CD3⁺ T cells as well as the absolute number of CD4⁺ and CD8⁺ T cells in PBMCs.

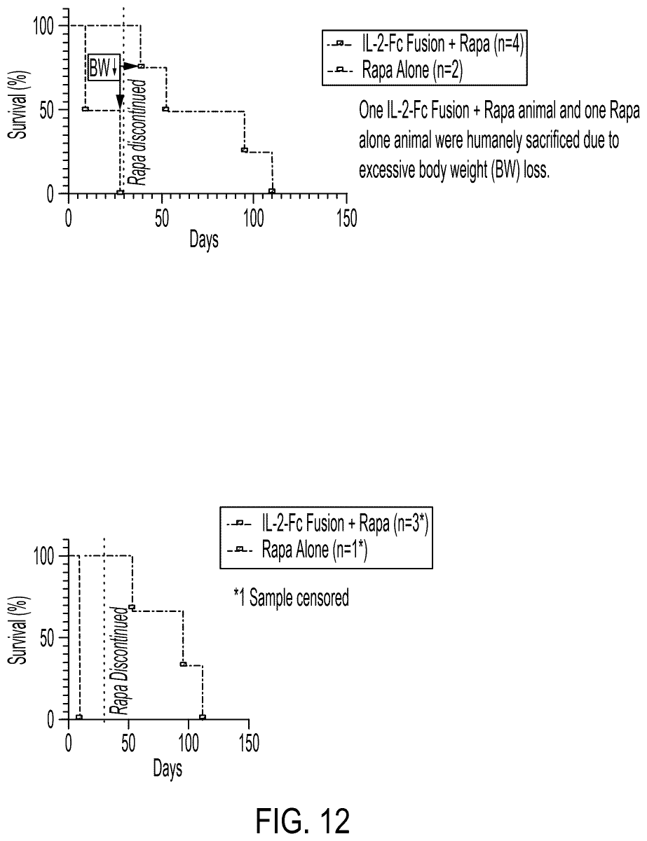

The survival for each testing group is also summarized in FIG. 12. One monkey in each testing group was sacrificed early due to excessive body weight loss and was censored from the bottom survival graph in FIG. 12.

Overall, this example showed that administration of an exemplary alpha-biased IL-2 variant can improve long-term kidney transplant outcomes even when treatment is discontinued.

### EQUIVALENTS AND SCOPE

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

1. A method of prophylaxis for kidney rejection in a recipient receiving a kidney transplant, comprising administering to the recipient an alpha-biased IL-2 variant in conjunction with the kidney transplant.

2. A method of improving a kidney transplant in a recipient,
comprising administering to the recipient an alpha-biased IL-2 variant in conjunction with the kidney transplantation at a therapeutically effective amount,
wherein the improvement in the kidney transplant results in prolonged stability of the kidney transplant, survival of the recipient, and/or reduced rejection symptoms as compared to a control.

3. The method of claim 2, wherein the control is historical data, a comparable recipient without the administration of the alpha-biased IL-2 variant, or a comparable recipient receiving the standard of care.

4-10. (canceled)

11. The method of claim 2, wherein the alpha-biased IL-2 variant is administered at least once, at least twice, or at least three times prior to the kidney transplantation.

12. The method of claim 2, wherein the alpha-biased IL-2 variant is administered at least once, at least twice, at least three times, at least four times, at least five times, or at least 6 times subsequent to the kidney transplantation.

13. A method of conditioning a kidney transplant recipient,
comprising administering to the recipient an alpha-biased IL-2 variant
wherein the alpha-biased IL-2 variant is administered prior to the kidney transplant at a therapeutically effective amount.

14. The method of claim 13, further comprising administering the alpha-biased IL-2 variant subsequent to the kidney transplantation.

15. The method of claim 2, wherein the administration of the alpha-biased IL-2 variant results in the kidney transplant to be stable for at least 50 days.

16-20. (canceled)

21. The method of claim 2, wherein the method comprises discontinuing the administration of the alpha-biased IL-2 variant within 30 days of the kidney transplantation.

22. The method of claim 2, wherein the alpha-biased IL-2 variant comprises an amino acid substitution H16L, H16N, V69, Q74, I92S, D84V, and/or S87R.

23. (canceled)

24. The method of claim 22, wherein the alpha-biased IL-2 variant comprises amino acid substitutions H16L, V69A, and Q74P.

25. The method of claim 2, wherein the alpha-biased IL-2 variant comprises an amino acid sequence that is at least 90% identical to SEQ ID NO: 4.

26. The method of claim 2, wherein the alpha-biased IL-2 variant is fused to a half-life extension domain, wherein the half-life extension domain is an Fc domain, a human serum albumin, or an albumin binding domain.

27-28. (canceled)

29. The method of claim 26, wherein the Fc domain comprises an IgG1 Fc domain, an IgG2 Fc domain, and IgG3 Fc domain, or an IgG4 Fc domain.

30. (canceled)

31. The method of claim 26, wherein the Fc domain comprises an amino acid substitution of N297G.

32. The method of claim 26, wherein the alpha-biased IL-2 variant is fused to the half-life extension domain via a linker.

33. (canceled)

34. The method of claim 26, wherein the alpha-biased IL-2 fused to the Fc domain comprises an amino acid sequence that is at least 90% identical to SEQ ID NO: 27.

35-47. (canceled)

48. The method of claim 2, wherein the administration of the alpha-biased IL-2 variant results in a successful taper and/or withdrawal of the standard care.

49-54. (canceled)

55. A method of prophylaxis for kidney rejection in a recipient receiving a kidney transplant, comprising administering to the recipient an IL-2 variant in conjunction with the kidney transplant, wherein the IL-2 variant comprises one or more amino acid substitutions selected from Table 1.

56-60. (canceled)

61. The method of claim 55, wherein the administering of the IL-2 variant improves the kidney transplant in the recipient as compared to a control.

62-72. (canceled)


SEQUENCE LISTING (27 sequences)

SEQ ID NO: 1 - protein, 133 aa, synthetic construct [sequence omitted]

SEQ ID NO: 2 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct [sequence omitted]

SEQ ID NO: 4 - protein, 133 aa, synthetic construct
APTSSSTKKTQLQLELLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 5 - protein, 133 aa, synthetic construct
APTSSSTKKTQLQLENLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 6 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISNINVSVLELKGSETTFMCEYADETATIVEFLNRWITFSQSIISTLT

SEQ ID NO: 7 - protein, 133 aa, synthetic construct
APASSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 8 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 5 aa, synthetic construct
GGGGS

SEQ ID NO: 13 - protein, 20 aa, synthetic construct
GGGGSGGGGSGGGGSGGGGS

SEQ ID NO: 14 - protein, 5 aa, synthetic construct
GGGSA

SEQ ID NO: 15 - protein, 7 aa, synthetic construct
AEAAAKA

SEQ ID NO: 16 - protein, 36 aa, synthetic construct
AGSGGSGGSGGSPVPSTPPTNSSSTPPTPSPSASGS

SEQ ID NO: 17 - protein, 44 aa, synthetic construct
AGSGGSGGSGGSPVPSTPPTPSPSTPPTPSPSGGSGNSSGSGGS

SEQ ID NO: 18 - protein, 48 aa, synthetic construct
AGSGNSSGSGGSGGSGNSSGSGGSPVPSTPPTPSPSTPPTPSPSASGS

SEQ ID NO: 19 - protein, 24 aa, synthetic construct
AEAAAKEAAAKEAAAKEAAAKAGS

SEQ ID NO: 20 - protein, 25 aa, synthetic construct
GTTPNPPASSSTTGSSTPTNPPAGS

SEQ ID NO: 21 - protein, 37 aa, synthetic construct
AGSPGAGNGGNNGGNPPPPTTTTSSAPATTTTASAGS

SEQ ID NO: 22 - protein, 22 aa, synthetic construct
GGGSAGGGSAGGGSAGGGSAGS

SEQ ID NO: 23 - protein, 380 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 380 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 380 aa, synthetic construct
APTSSSTKKTQLQLELLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 380 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 380 aa, synthetic construct
APTSSSTKKTQLQLELLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK